(19) United States
(12) Patent Application Publication
(10) Pub. No.: US 2019/0307843 A1
(43) Pub. Date: Oct. 10, 2019

(54) SINGLE DOSE METHODS FOR PREVENTING AND TREATING FUNGAL INFECTIONS

(71) Applicant: Cidara Therapeutics, Inc., San Diego, CA (US)

(72) Inventors: Kenneth BARTIZAL, La Jolla, CA (US); Paul DARUWALA, Del Mar, CA (US); Jeffrey B. LOCKE, La Jolla, CA (US); Voon ONG, San Diego, CA (US); Taylor SANDISON, Encinitas, CA (US)

(21) Appl. No.: 16/346,424

(22) PCT Filed: Oct. 30, 2017
(86) PCT No.: PCT/US17/59046

§ 371 Date: Apr. 30, 2019

Related U.S. Application Data
(60) Provisional application No. 62/415,974, filed on Nov. 1, 2016.
(60) Provisional application No. 62/418,736, filed on Nov. 7, 2016.
(60) Provisional application No. 62/418,982, filed on Nov. 8, 2016.
(60) Provisional application No. 62/436,632, filed on Dec. 20, 2016.
(60) Provisional application No. 62/504,752, filed on May 11, 2017.
(60) Provisional application No. 62/531,722, filed on Jul. 12, 2017.
(60) Provisional application No. 62/541,467, filed on Aug. 4, 2017.
(60) Provisional application No. 62/560,851, filed on Sep. 20, 2017.
(60) Provisional application No. 62/566,845, filed on Oct. 2, 2017.

(51) Int. Cl. A61K 38/17; A61P 31/10; A61K 9/00; A61L 29/08

(52) U.S. Cl. CPC A61K 38/1774; A61P 31/10; A61L 29/085; A61K 9/0019; A61K 9/0053

(57) ABSTRACT

Provided herein are methods for treating, mitigating, or preventing fungal infections or related conditions thereto in a human subject in need thereof. The methods include the administration of a single dose of a pharmaceutical composition comprising or consisting of CD101 and any pharmaceutically acceptable excipients, wherein the single dose treatment substantially reduces or eliminates the fungal infection.

### BACKGROUND

This invention features methods for the treatment of fungal infections and conditions related thereto.

Fungal infections, such as those caused by Candida and Aspergillus, can be serious and life-threatening infections that represent a significant public health issue, particularly in highly vulnerable populations including the elderly, post-surgical, critically ill, and other hospitalized patients with serous medical conditions. Because of increasing resistance to existing antifungal drugs, there is an urgent need to develop new and more effective antifungal agents to treat these serious infections. Echinocandins are members of a leading class of antifungal agents for the treatment of fungal infections. These compounds target the cell wall by preventing the production of 1,3-β-D-glucan through inhibition of the catalytic subunit of 1,3-β-D-glucan synthase enzyme complex. The three echinocandins approved by the U.S. Food and Drug Administration for the treatment of fungal infections (caspofungin, anidulafungin, and micafungin) are available only in intravenous formulations. Further, these antifungal agents must be administered daily over multiple days, making it challenging to transition patients to a home setting. Further, failure to comply with this multi-day regimen may contribute to the rise in reports of drug-resistant fungal infections. Thus, there is a need in the art for improved methods of preventing and treating fungal infections.

### SUMMARY OF THE INVENTION

The invention is directed to methods of treating fungal infections in a human subject by administering a single dose of CD101 in salt or neutral form.

In a first aspect, the invention features a method of treating a condition or disorder in a human subject. This method includes or consists of administering to the subject a single dose of a pharmaceutical composition including CD101 salt, or a neutral form thereof, and one or more pharmaceutically acceptable excipients, wherein the single dose includes an amount of the CD101 salt, or a neutral form thereof, sufficient to treat the condition or disorder.

In a related aspect, the invention features a method of preventing a condition or disorder in a human subject. This method includes or consists of administering to the subject a single dose of a pharmaceutical composition including CD101 salt, or a neutral form thereof, and one or more pharmaceutically acceptable excipients, wherein the single dose includes an amount of the CD101 salt, or a neutral form thereof, sufficient to prevent the condition or disorder.

In either the first or second aspects, the single dose can be administered orally. In one particular embodiment, the single dose includes from 50 mg to 1200 mg (e.g., 100±50 mg, 200±50 mg, 300±50 mg, 400±50 mg, 500±50 mg, 600±50 mg, 700±50 mg, 750±50 mg, 800±100 mg, 900±100 mg, 1000±100 mg, or 1100±100 mg) of the CD101 salt, or a neutral form thereof, administered orally.

In either the first or second aspects, the single dose can be administered intravenously. In one embodiment, the single dose includes from 50 mg to 1200 mg (e.g., 100±50 mg, 200±50 mg, 300±50 mg, 400±50 mg, 500±50 mg, 600±50 mg, 700±50 mg, 750±50 mg, 800±100 mg, 900±100 mg, 1000±100 mg, or 1100±100 mg) of the CD101 salt, or a neutral form thereof, administered intravenously.

In either the first or second aspects, the single dose can be administered subcutaneously. In one embodiment, the single dose includes from 50 mg to 1200 mg (e.g., 100±50 mg, 200±50 mg, 300±50 mg, 400±50 mg, 500±50 mg, 600±50 mg, 700±50 mg, 750±50 mg, 800±100 mg, 900±100 mg, 1000±100 mg, or 1100±100 mg) of the CD101 salt, or a neutral form thereof, administered subcutaneously.

In either the first or second aspects, the single dose can be administered intramuscularly. In one embodiment, the single dose includes from 50 mg to 1200 mg (e.g., 100±50 mg, 200±50 mg, 300±50 mg, 400±50 mg, 500±50 mg, 600±50 mg, 700±50 mg, 750±50 mg, 800±100 mg, 900±100 mg, 1000±100 mg, or 1100±100 mg) of the CD101 salt, or a neutral form thereof, administered intramuscularly.

In any of the above embodiments, the disease or condition treated can be selected from the group consisting of candidemia, invasive candidiasis, onychomycosis, inflammatory bowel disease, aspergillosis, and a Pneumocystis infection.

In any of the above embodiments, the disease or condition treated can be a fungal infection. In one embodiment, the fungal infection is a Candida infection. In another embodiment, the fungal infection is an Aspergillus infection. In some embodiments, the fungal infection is a Pneumocystis infection.

In any of the above embodiments, the administration of the single dose can substantially eliminate or prevent a fungal infection.

In an embodiment of any of the above methods, the subject does not receive any concurrent antifungal treatment. Alternatively, the subject does not receive any antifungal treatment within 21 days, 28 days, 35 days, 42 days, or 56 days following the administration of the pharmaceutical composition.

In any of the above embodiments, the pharmaceutical composition can consist of the CD101 salt, or a neutral form thereof, and the one or more pharmaceutically acceptable excipients.

In any of the above methods, the CD101 could be substituted with compound 2 in salt or neutral form (described herein) or a compound described in U.S. Pat. No. 9,217,014, incorporated herein by reference. For example, CD101 could be substituted with a compound described in U.S. Pat. No. 9,217,014 selected from the group consisting of compound 6, compound 7, compound 12, compound 15, compound 17, compound 23, compound 24, and pharmaceutically acceptable salts thereof.

In another aspect, the invention features a method of preventing or treating a biofilm in a subject. The method includes administering to the subject a pharmaceutical composition comprising CD101 salt, or a neutral form thereof, and one or more pharmaceutically acceptable excipients.

In some embodiments of this aspect, the biofilm in the subject is a Candida biofilm (e.g., Candida albicans biofilm or a Candida auris biofilm). In some embodiments, the biofilm is attached to a mucous membrane of the subject.

In another aspect, the invention features a method of preventing biofilm growth on a catheter or killing a biofilm attached to a catheter comprising submerging the catheter in an aqueous solution comprising CD101 salt, or a neutral form thereof, or running an aqueous solution comprising CD101 salt, or a neutral form thereof, through the lumen of the catheter.

In some embodiments of this aspect, the biofilm on the catheter is a Candida biofilm (e.g., Candida albicans biofilm or a Candida auris biofilm).

### Definitions

For the purpose of the present invention, the following abbreviations and terms are defined below.

As used herein, the term “about” refers to a range of values that is ±10% of specific value. For example, “about 150 mg” includes ±10% of 150 mg, or from 135 mg to 165 mg. Such a range performs the desired function or achieves the desired result. For example, “about” may refer to an amount that is within less than 10% of, within less than 5% of, within less than 1% of, within less than 0.1% of, and within less than 0.01% of the stated amount. Other than in the examples, or where otherwise indicated, all numbers expressing quantities of ingredients or reaction conditions used herein should be understood as modified in all instances by the term “about.”

As used herein, the terms “associated with” and “related to” refer to symptoms, conditions, diseases, syndromes, or disorders that may be diagnosed in an individual who has developed or is at risk of developing of fungal infections. For example, the fungal infection may be a causal factor for a related condition, a factor that exacerbates a related condition without necessarily being causal, or a symptom or outcome of a related condition. Further, the fungal infection may occur at any point in time relative to the related condition (e.g., before, concomitant with, or after onset of the related condition).

As used herein, the term “CD101 salt” refers to a salt of the compound of Formula 1. CD101 has a structure (below) in which the tertiary ammonium ion positive charge of CD101 is balanced with a negative counterion (e.g., an acetate) in its salt form. The structure of CD101 is depicted below.

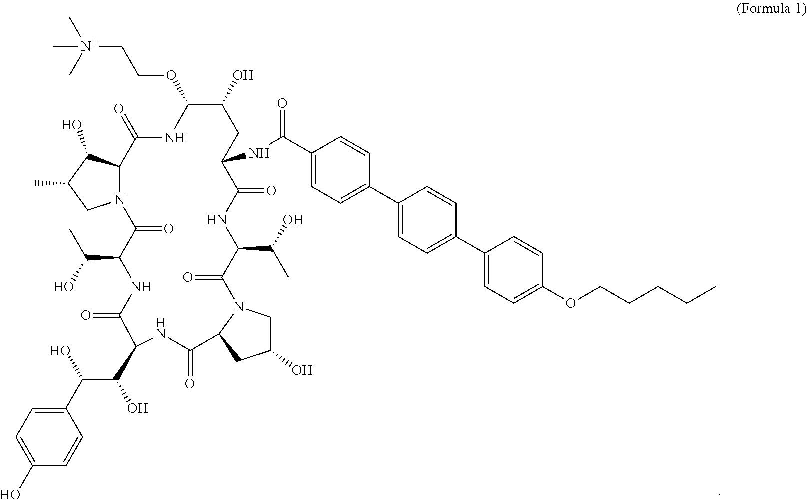 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]

As used herein, the term “compound 2” refers to a salt of the compound of Formula 2, or a neutral form thereof. Compound 2 has a structure (below) in which the tertiary ammonium ion positive charge of the compound in Formula 2 is balanced with a negative counterion (e.g., an acetate) in its salt form. The structure of compound 2 is depicted below.

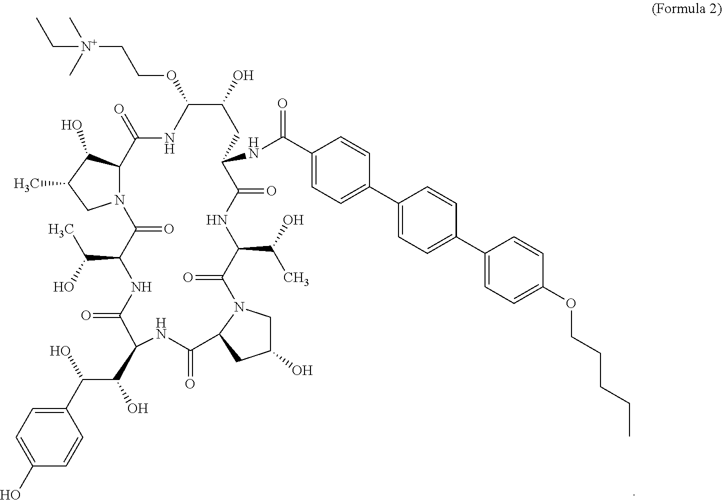 [SMILES: CCCCCOc1ccc(-c2ccc(-c3ccc(C(=O)N[C@H]4C[C@@H](O)[C@@H](OCC[N+](C)(C)CC)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]

CD101 and compound 2 are semi-synthetic echinocandin compounds that inhibits the synthesis of 1,3-β-D-glucan, an essential component of the fungal cell wall of yeast forms of Candida species and regions of active cell growth of Aspergillus hyphae. The synthesis of 1,3-β-D-glucan is dependent upon the activity of 1,3-β-D-glucan synthase, an enzyme complex in which the catalytic subunit is encoded by FKS1, FKS2, and FKS3 genes. Inhibition of this enzyme results in rapid, concentration-dependent, fungicidal activity for Candida spp. As used herein, the term “CD101 neutral form” includes the zwitterionic forms of CD101 in which the compound of Formula 1 or 2 has no net positive or negative charge. The zwitterion is present in a higher proportion in basic medium (e.g., pH 9) relative to CD101, or a salt thereof. In some embodiments, the zwitterion may also be present in its salt form.

As used herein, the term “CD101 neutral form” or “compound 2 neutral form” includes the zwitterionic forms of CD101 in which the compound of Formula 1 or 2 has no net positive or negative charge. The zwitterion is present in a higher proportion in basic medium (e.g., pH 9) relative to CD101, compound 2, or a salt thereof. In some embodiments, the zwitterion may also be present in its salt form.

The “colonization” of a host organism includes the non-transitory residence of a fungi in or on any part of the body of a human subject. As used herein, “reducing colonization” of a pathogenic fungi (opportunistic or non-opportunistic) in any microbial niche includes a reduction in the residence time of the pathogen and/or a reduction in the number (or concentration) of the pathogen in the colonized part of the subject's body.

By “concurrent antifungal treatment” is meant any additional dose of an antifungal agent (e.g., CD101 or another antifungal agent) administered within 12 hours, 24 hours, 2 days, 3 days, 4 days, 5 days, 6, days, or 1 week before or after administration of the single dose of CD101) that would confer therapeutic benefits (e.g., be systemically active) in the treatment of the targeted fungal infection at the same time that CD101 is at a therapeutically effective concentration in the subject. In some instances, the single dose treatment is not combined with any other antifungal treatment within 1-21 days before or after administration. For example, a single dose of CD101 may be administered (e.g., orally, intravenously, subcutaneously, or intramuscularly) to a subject with a fungal infection and the single dose effectively treats the fungal infection without necessitating additional antifungal treatments before, concurrently, or after the single dose treatment with CD101.

“Dysbiosis” or “microbial dysbiosis” refers to a state of the microbiota (e.g., fungi and bacteria) on or in any part of the body of a human subject in which the normal diversity and/or function of the ecological network is disrupted. For example, this disrupted state can be due to a decrease in fungal diversity, an overgrowth of one or more fungal pathogens or pathobionts, or a shift to an ecological microbial network that no longer provides an essential function to the host subject, and therefore no longer promotes health. As used herein, the term “pathobiont” or “opportunistic pathogen” refers to symbiotic fungi able to cause disease only when certain genetic and/or environmental conditions are present in a subject.

By “dose” is meant the amount of CD101 (Formula 1) administered to the human subject.

The term “dosage form” or “unit dosage form,” as used herein, refers to physically discrete units suitable as unitary dosages, such as a pill, tablet, caplet, hard capsule or soft capsule, each unit containing a predetermined quantity of a drug.

By “effective” amount is meant the amount of drug required to treat or prevent a fungal infection or a disease associated with a fungal infection. The effective amount of drug used to practice the methods described herein for therapeutic or prophylactic treatment of conditions caused by or contributed to by a fungal infection varies depending upon the manner of administration, the age, body weight, and general health of the subject. Ultimately, the attending physician will decide the appropriate amount and dosage regimen. Such amount is referred to as an “effective” amount.

By “infection” or “fungal infection” is meant a microbial dysbiosis characterized by overgrowth or colonization of any part of the body of a human subject by one or more species of fungi (e.g., fungal pathogens or opportunistic pathogens), reduction of which may provide benefit to the host. For example, the infection may include the excessive growth of or colonization by fungal species that are normally present in or on the body of a human subject, or the infection may include colonization by fungal species that are not normally present in or on the body of a human subject. In some instances, the infection may include colonization of a part of the body by a fungus that is indigenous to some parts of the human body (e.g., GI tract) but is detrimental when found in other parts of the body (e.g., tissues beyond the GI tract). More generally, an infection can be any situation in which the presence of a microbial population(s) is damaging to a host body.

As used herein, the term “microbiota” refers to the community of microorganisms that occur (sustainably or transiently) in and on the human body.

As used herein, the term “biofilm” refers to a three-dimensional structure composed of heterogenous fungi (e.g., Candida) and hyphae that can attach to various surfaces, e.g., a mucous membrane or the inside of a catheter. Biofilms can form on the surfaces of medical devices and cause biofilm device-associated infections. For example, having a biofilm on an indwelling device, e.g., a vascular catheter, can cause life-threatening infections.

As used herein, “parenteral administration” and “administered parenterally” refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

As used herein, the term “salt” refers to any pharmaceutically acceptable salt, such as a non-toxic acid addition salt, metal salt, or metal complex, commonly used in the pharmaceutical industry. Acid addition salts include organic acids, such as acetic, lactic, palmoic, maleic, citric, cholic acid, capric acid, caprylic acid, lauric acid, glutaric, glucuronic, glyceric, glycocolic, glyoxylic, isocitric, isovaleric, lactic, malic, oxalo acetic, oxalosuccinic, propionic, pyruvic, ascorbic, succinic, benzoic, palmitic, suberic, salicylic, tartaric, methanesulfonic, toluenesulfonic, and trifluoroacetic acids, and inorganic acids, such as hydrochloric acid, hydrobromic acid, sulfuric acid, and phosphoric acid. Representative alkali or alkaline earth metal salts include sodium, lithium, potassium, calcium, and magnesium, among others.

As used herein, a “single dose” or “single dose treatment” of CD101 (e.g., CD101 in salt or neutral form) refers to treatment (e.g., substantial elimination) of a fungal infection in a subject by administration of not more than one dose of a pharmaceutical composition including CD101 in salt or neutral form and one or more pharmaceutically acceptable carriers or excipients during a six week, 8 week, or 12 week period. Desirably, the single dose administration is sufficient to treat the fungal infection without requiring a “concurrent antifungal treatment.”

By “subject” or “patient” is meant a human. A human subject who is being treated for a fungal infection is one who has been diagnosed by a medical practitioner as the case may be as having such a condition. Diagnosis may be performed by any suitable means. One in the art will understand that subjects of the invention may have been subjected to standard tests or may have been identified, without examination, as one at high risk due to the presence of one or more risk factors, such as age, genetics, or family history.

As used herein, the term “substantially eliminates” a fungal infection refers to reducing colonization (see definition above) by one or more opportunistic or non-opportunistic pathogenic fungi (e.g., by about 1%, 2%, 5%, 10%, 20%, 30%, 40%, 50%, 60%, 70%, 80%, 90%, or %100 relative to a starting amount) in one or more parts of the body in an amount sufficient to restore a normal fungal population (e.g. approximately the amount found in a healthy individual) and/or allow benefit to the individual (e.g., reducing colonization in an amount sufficient to sustainably resolve symptoms). For example, a fungal infection can be caused by overgrowth of an opportunistic pathogen that is normally present on the human body but has grown above healthy levels, in which case the infection may be eliminated by reducing fungal species to a level typically found in a healthy individual without necessarily eliminating the fungal species. Alternatively, for example, a fungal pathogen or opportunistic pathogen may colonize a portion of the body in which it does not typically reside and thus, the infection is treated when the fungal population is eradicated.

As used herein, the term “substantially prevents’ refers to preventing increased colonization by one or more opportunistic or non-opportunistic pathogenic fungi (e.g., by about 1%, 2%, 5%, 10%, 20%, 30%, 40%, 50%, 60%, 70%, 80%, 90%, %100, or more than %100 relative to a starting amount) in one or more parts of the body in an amount sufficient to maintain a normal fungal population (e.g., approximately the amount found in a healthy individual), prevent the onset of a fungal infection, and/or prevent symptoms or conditions associated with infection. For example, subjects may receive prophylaxis treatment to substantially prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised subjects (e.g., subjects with cancer, with HIV/AIDS, or taking immunosuppressive agents), or in subjects undergoing long term antibiotic therapy.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

Other features and advantages of the invention will be apparent from the following detailed description, the drawings, and the claims.

### BRIEF DESCRIPTION OF THE DRAWINGS

FIG. 1 is a graph showing reductions in kidney colony forming units following CD101 subcutaneous administration.

FIG. 2 is a graph showing total CD101 exposure following intravenous and subcutaneous administration.

FIG. 3 is a graph showing percent survival over time in mice infected with Aspergillus fumigatus and treated with 2 mg/kg CD101 (IV or IP).

FIG. 4 is a table showing the activity of various antifungal agents against Candida auris clinical isolates.

FIG. 5 is a graph showing plasma levels of CD101 in two cynomolgus monkeys over 10 days after a single 30 mg/kg dose administered subcutaneously.

FIG. 6 is a graph showing percent survival over time in mice infected with Candida auris and treated with 20 mg/kg CD101 (IP), 20 mg/kg fluconazole (PO), or 0.3 mg/kg amphotericin B (IP).

FIGS. 7A and 7B are bar graphs showing the effect of CD101 (0.25 or 1 μg/ml) (FIG. 7A) and fluconazole (1 or 4 μg/ml) (FIG. 7B) on metabolic activity of adhesion phase C. albicans biofilms compared to untreated control.

FIGS. 8A-8E are confocal scanning laser micrographs showing the effect of CD101 and fluconazole on adhesion phase C. albicans biofilms (prevention): top-down three-dimensional view (top panels) and side-views (bottom panels) of biofilms formed by C. albicans treated with: no drug (control; FIG. 8A), 0.25 μg/ml CD101 (FIG. 8B), 1 μg/ml CD101 (FIG. 8C), 1 μg/ml fluconazole (FIG. 8D), and 4 μg/ml fluconazole (FIG. 8E).

FIGS. 8F and 8G are bar graphs showing the thickness of C. albicans biofilms exposed to CD101 (FIG. 8F) and fluconazole (FIG. 8G).

FIGS. 9A and 9B are bar graphs showing the effect of CD101 (0.25 or 1 μg/ml) (FIG. 9A) and fluconazole (1 or 4 μg/ml) (FIG. 9B) on metabolic activity of mature phase C. albicans biofilms compared to untreated control.

FIGS. 10A-10E are confocal scanning laser micrographs showing the effect of CD101 and Fluconazole on mature phase C. albicans biofilms (treatment): Top-down three-dimensional view (top panels) and side-view (bottom panels) of biofilms exposed to: no drug (FIG. 10A), 0.25 μg/ml CD101 (FIG. 10B), 1 μg/ml CD101 (FIG. 100), 1 μg/ml fluconazole (FIG. 10D), and 4 μg/ml fluconazole (FIG. 10E). Arrows show bulged/broken cells.

FIGS. 10F and 10G are bar graphs showing thickness of C. albicans biofilms exposed to: CD101 (FIG. 10F) and fluconazole (FIG. 10G).

FIGS. 11A-11F are images showing the temporal effect of CD101 (0.25 μg/ml) on formation of C. albicans biofilms. Images were captured immediately from 0 h and followed up to 16 h for biofilms treated with: no drug (FIGS. 11A and 11B), CD101 at low magnification, ×20 (FIGS. 11C and 11D), and CD101 at high magnification, ×63 (FIGS. 11E and 11F). Arrows show bulging, deformed, and broken cells.

FIGS. 12A and 12B are images showing the temporal effect of CD101 (0.25 μg/ml) on 3 h formed C. albicans biofilms. CD101 was added after 3 h biofilm formation and images were captured immediately after adding CD101 (FIG. 12A) and followed up to 16 h (FIG. 12B), magnification, ×63. Arrows show bulging, deformed, and broken cells.

FIG. 13 is a graph showing kidney fungal burden in neutropenic mice prophylactically treated with a single subcutaneous administration of CD101 and infected with Candida albicans.

FIG. 14A is a graph showing percent survival over time in neutropenic mice prophylactically treated with CD101 and infected with Aspergillus fumigatus.

FIG. 14B is a graph showing the pharmacokinetic profile of CD101 in mice following a 10-mg/kg subcutaneous dose injection.

FIG. 14C is a graph showing a correlation between free drug plasma concentration at time of infection over MIC (0.03 μg/mL) with higher free drug plasma concentration generating greater CFU reduction.

FIG. 15 shows an outline of the study design for Example 11.

FIG. 16A is a line graph showing the average group weight of rats with vulvovaginal candidiasis throughout the study. Arrows on x-axis indicate the estradiol treatment days.

FIG. 16B is a line graph showing the average group weights of rat with vulvovaginal candidiasis throughout the study relative to weight on day of infection (Day 0). Arrows on x-axis indicate the estradiol treatment days.

FIG. 17 is a graph showing the pharmacokinetic profile of CD101 in a rat model of vulvovaginal candidiasis (VVC) following a 10 mg/kg intravenous and subcutaneous dose injection.

FIG. 18 is a scatterplot showing vaginal lavage burden Day +1 (24 h) post infection/prior to treatments following localized vaginal infection with C. albicans 529L.

FIG. 19 is a scatterplot showing vaginal lavage burden Day +2 (48 h) post infection.

FIG. 20 is a scatterplot showing vaginal lavage burden Day +3 (72 h) post infection.

FIG. 21 is a scatterplot showing vaginal lavage burden Day +5 (120 h) post infection.

FIG. 22 is a scatterplot showing vaginal lavage burden Day +7 (168 h) post infection.

FIG. 23 is a scatterplot showing vaginal lavage burden Day +9 (216 h) post infection.

FIG. 24A is a bar graph showing the mean daily vaginal lavage burden of the rats in each group over duration of the study following localized vaginal infection with C. albicans 529L and administration with vehicle, CD101, or fluconazole (error bars are geometric standard deviation).

FIG. 24B is a scatterplot showing the daily vaginal lavage burden of each rat over duration of study following localized vaginal infection with C. albicans 529L and administration with vehicle, CD101, or fluconazole.

FIG. 24C is a line graph showing the daily vaginal lavage burden of the rats in each group over duration of study following localized vaginal infection with C. albicans 529L and administration with vehicle, CD101, or fluconazole (error bars are geometric standard deviation).

FIG. 25A is a bar graph showing the geometric mean terminal vaginal tissue burden (vagina, uterus, and uterine horns) Day +9 (216 h) post infection (error bars are geometric standard deviation).

FIG. 25B is a scatterplot showing terminal vaginal tissue burden (vagina, uterus, and uterine horns) Day +9 (216 h) post infection.

FIG. 26 shows the plasma concentrations of CD101 in mice following single IP doses of CD101. Samples were obtained at seven time points over 72 hours. Each symbol represents the mean and standard deviation from three mice. Cₘₐₓ represents the peak concentration, AUC is from 0 to infinity, and T_{1/2} the beta elimination half-life.

FIG. 27A shows CD101 dose-response curves against C. albicans. Each symbol represents the mean and standard deviation from three mice. The horizontal dashed-line at 0 represents the burden of organisms in the kidneys of mice at the start of therapy. Data points below the line represent cidal activity and points above the line represent net growth.

FIG. 27B shows CD101 dose-response curves against C. glabrata.

FIG. 27C shows CD101 dose-response curves against C. parapsilosis.

FIG. 28A shows the relationship between total and free drug AUC/MIC and treatment effect for C. albicans. AUC is measured as the total or free AUC over the full treatment course (168 h). Each symbol represents the mean fungal burden from three mice. The horizontal dashed-line at 0 represents the burden of organisms in the kidneys of mice at the start of therapy. Data points below the line represent cidal activity and points above the line represent net growth. The curved line through the data is the best fit line based on the hill equation and the co-efficient of determination (R2) is shown for each organism group.

FIG. 28B shows the relationship between total and free drug AUC/MIC and treatment effect for C. glabrata.

FIG. 28C shows the relationship between total and free drug AUC/MIC and treatment effect for C. parapsilosis.

FIG. 29A shows the relationship between 24 h average free drug AUC/MIC (fAUC/MIC) and treatment effect for C. albicans. Each symbol represents the mean fungal burden from three mice. The horizontal dashed-line at 0 represents the burden of organisms in the kidneys of mice at the start of therapy. Data points below the line represent cidal activity and points above the line represent net growth. The curved line through the data is the best fit line based on the hill equation and co-efficient of determination (R2) is shown for each organism group. Also shown is the maximum effect (Emax), 50% maximum effect (ED50), and slope of the line (N).

FIG. 29B shows the relationship between 24 h average free drug AUC/MIC (fAUC/MIC) and treatment effect for C. glabrata.

FIG. 29C shows the relationship between 24 h average free drug AUC/MIC (fAUC/MIC) and treatment effect for C. parapsilosis.

FIG. 30 shows animal weights following infection with A. fumigatus strain AF293. The arrows on the x-axis show the immunosuppression day.

FIG. 31 shows a Kaplan Meir plot of survival for a murine model of pulmonary aspergillosis treated with CD101 at 5 mg/kg, 10 mg/kg, or 20 mg/kg zero, one, three, or five days pre-infection or treated with the comparator micafungin at 2 mg/kg zero or one days pre-infection.

FIG. 32 shows unbound CD101 plasma concentrations in healthy human adults relative to antifungal activity.

FIG. 33 shows the tissue distribution of CD101 and associated half-life of CD101 in various organs in rats following a 5 mg/kg IV CD101 dose.

FIG. 34 shows the efficacy of 5 mg/kg CD101 SC and amphotericin B in the survival of neutropenic ICR female mice infected with A. fumigatus (ATCC 13073). An asterisk (*) indicates a 50 percent or more (50%) increase in the survival rate compared to the vehicle control group.

FIG. 35 shows the efficacy of 10 mg/kg CD101 SC and amphotericin B in the survival of neutropenic ICR female mice infected with A. fumigatus (ATCC 13073). An asterisk (*) indicates a 50 percent or more (50%) increase in the survival rate compared to the vehicle control group.

FIG. 36 shows the efficacy of 20 mg/kg CD101 SC and amphotericin B in the survival of neutropenic ICR female mice infected with A. fumigatus (ATCC 13073). An asterisk (*) indicates a 50 percent or more (50%) increase in the survival rate compared to the vehicle control group.

FIG. 37 is a graph showing CD101 plasma and epithelial lining fluid (ELF) concentration-time profiles following CD101 IP 20 mg/kg administration.

FIG. 38 is a graph showing the survival rate of mice given prophylaxis CD101 IP 20 mg/kg or posaconazole (PO; 2 or 10 mg/kg) as a single dose one day prior to infection in pulmonary aspergillosis.

FIG. 39 is a graph showing CD101 plasma (total- and free-drug) and ELF concentration-time profiles following CD101 IP 20 mg/kg administration.

FIG. 40 is a graph showing the average group weight of mice throughout the study relative to mice weight on Day −4 pre-infection.

FIG. 41 is a graph showing a Kaplan Meier plot of survival for a murine model of pulmonary aspergillosis treated with a single dose of CD101 IP at 20 mg/kg and 60 mg/kg one day pre-infection or treated with the comparator posaconazole 2 mg/kg and 10 mg/kg one day pre-infection.

FIG. 42 is a graph showing the geometric mean terminal lung burden of a murine model of pulmonary aspergillosis treated with a single dose of CD101, micafungin, or posaconazole.

FIG. 43 is a graph showing the geometric mean terminal lung burden of a murine model of pulmonary aspergillosis treated with a single dose of CD101 or micafungin.

### DETAILED DESCRIPTION

Provided herein are methods for treating, mitigating, or preventing a fungal infection or related conditions thereto in a human subject in need thereof. The methods include the administration of a single dose of a pharmaceutical composition including or consisting of CD101 and any pharmaceutically acceptable excipients, wherein the single dose treatment substantially reduces or eliminates the fungal infection.

### I. Treatment Applications

The invention features methods for treating, mitigating, or preventing a fungal infection in a human subject in need thereof, wherein the fungal infection is associated with a disruption in the levels or composition of fungal species (e.g., Candida infection, Aspergillus infection, or Pneumocystis infection) in or on one or more body regions or tissues of the host subject. Further, the method can be used to treat symptoms, manifestations, conditions, or diseases associated with a fungal infection. In some instances, the fungal infection can be a primary diagnosis (e.g., the root cause of a symptom or condition), secondary to another condition or disease (e.g., a symptom or outcome of another condition); or a combination thereof. The fungal infection may be associated with one or more fungal species and/or colonization of the fungal species on one or more body regions or tissues of the host subject.

The methods of the invention can treat a fungal infection, for example, by reducing colonization by one or more opportunistic or non-opportunistic pathogenic fungi (e.g., by about 1%, 2%, 5%, 10%, 20%, 30%, 40%, 50%, 60%, 70%, 80%, 90%, or %100 relative to a starting amount) in one or more parts of the body in an amount sufficient to restore a normal fungal population (e.g. approximately the amount found in a healthy individual) and/or allow benefit to the individual (e.g., reducing colonization in an amount sufficient to sustainably resolve symptoms). For example, a fungal infection can be caused by overgrowth of an opportunistic pathogen that is normally present on the human body but has grown above healthy levels, in which case the infection may be eliminated by reducing fungal species to a level typically found in a healthy individual without necessarily eliminating the fungal species. Alternatively, for example, a fungal pathogen or opportunistic pathogen may colonize a portion of the body in which it does not typically reside and thus, the infection is treated when the fungal population is eradicated.

The methods of the invention can prevent a fungal infection, for example, by preventing an increased colonization by one or more opportunistic or non-opportunistic pathogenic fungi (e.g., prevent increase of about 1%, 2%, 5%, 10%, 20%, 30%, 40%, 50%, 60%, 70%, 80%, 90%, %100, or more than %100 relative to a starting amount) in one or more parts of the body in an amount sufficient to maintain a normal fungal population (e.g., approximately the amount found in a healthy individual), prevent the onset of a fungal infection, and/or prevent symptoms or conditions associated with infection. For example, subjects may receive prophylaxis treatment to substantially prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised subjects (e.g., subjects with cancer, with HIV/AIDS, or taking immunosuppressive agents), or in subjects undergoing long term antibiotic therapy.

In some instances, the methods provided herein can be used to treat a fungal infection associated with, or partially associated with, a fungal infection or fungal overgrowth localized to one or more portions of the human body. In some instances, the fungal species can be any species belonging to the phylum Ascomycota, Basidomycota, Chytridiomycota, Microsporidia, or Zygomycota. The fungal infection or overgrowth can include one or more fungal species, e.g., Candida albicans, C. tropicalis, C. parapsilosis, C. glabrata, C. auris, C. krusei, Saccharomyces cerevisiae, Malassezia globose, M. restricta, or Debaryomyces hansenii, Gibberella moniliformis, Alternaria brassicicola, Cryptococcus neoformans, Pneumocystis carinii, P. jirovecii, P. murina, P. oryctolagi, P. wakefieldiae, and Aspergillus clavatus. The fungal species may be considered a pathogen or an opportunistic pathogen. Further, the fungal species may be found indigenously in or on the human body (e.g., occurs sustainably regardless of level or concentration) or it can exist transiently in or on the human body.

In some instances, the fungal infection is caused by a fungus in the genus Candida (i.e., a Candida infection). For example, a Candida infection can be caused by a fungus in the genus Candida that is selected from the group consisting of C. albicans, C. glabrata, C. dubliniensis, C. krusei, C. auris, C. parapsilosis, C. tropicalis, C. orthopsilosis, C. guilliermondii, C. rugose, and C. lusitaniae. Candida infections that can be treated by the methods of the invention include, but are not limited to candidemia, oropharyngeal candidiasis, esophageal candidiasis, mucosal candidiasis, genital candidiasis, vulvovaginal candidiasis, rectal candidiasis, hepatic candidiasis, renal candidiasis, pulmonary candidiasis, splenic candidiasis, otomycosis, osteomyelitis, septic arthritis, cardiovascular candidiasis (e.g., endocarditis), and invasive candidiasis.

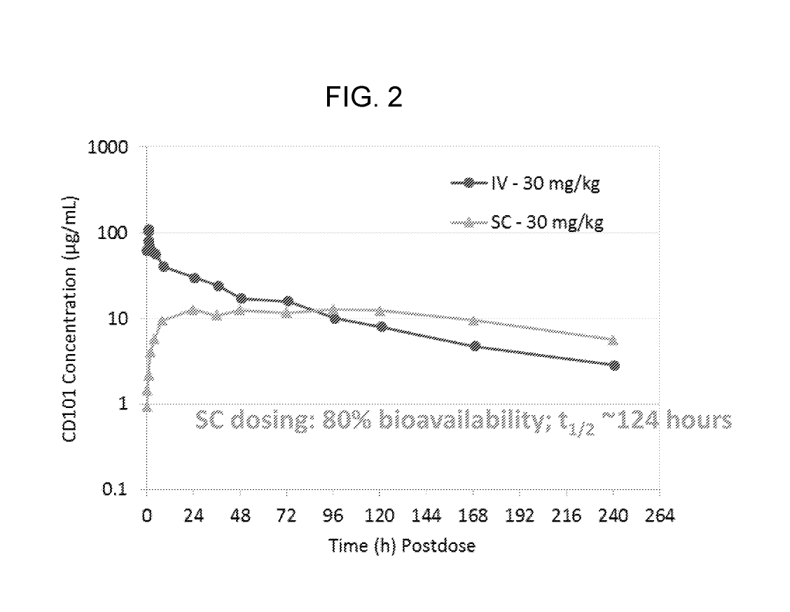

Clinical isolates of C. auris that may be treated or prevented by the methods described herein are described in the Examples section (e.g., Example 7) and also in Lee et al., J Clin Microbiol. 49:3139-42, 2011, Kathuria et al., J Clin Microbiol. 53:1823-30, 2015, and Vallabhaneni et al., MMWR Morb Mortal Wkly Rep. 65:1234-1237, 2016, each of which is incorporated by reference herein in its entirety. For example, FIG. 2 of Kathuria describes clinical isolates of C. auris which are shown in Table 1.

TABLE 1

| # | Clinical isolate |
|---|---|
| 1 | VPCI 717/P/14 |
| 2 | VPCI 462/P/14 |
| 3 | VPCI 1156/P/13 |
| 4 | VPCI 271/P/14 |
| 5 | VPCI 471/P/14 |
| 6 | VPCI 709/P/12 |
| 7 | VPCI 464/P/14 |
| 8 | VPCI 107/P/14 |
| 9 | VPCI 672/P/12 |
| 10 | VPCI 483/P/13 |
| 11 | VPCI 720/P/14 |
| 12 | VPCI 1132/P/13 |
| 13 | VPCI 512/P/14 |
| 14 | VPCI 249/P/14 |
| 15 | VPCI 553/P/14 |
| 16 | VPCI 1047/P/14 |
| 17 | VPCI 518/P/14 |
| 18 | VPCI 253/P/14 |
| 19 | VPCI 540/P/14 |
| 20 | VPCI 543/P/14 |
| 21 | VPCI 261/P/14 |
| 22 | VPCI 676/P/12 |
| 23 | VPCI 480/P/13 |
| 24 | VPCI 468/P/14 |
| 25 | VPCI 471/P/13 |
| 26 | VPCI 677/P/12 |
| 27 | VPCI 1131/P/13 |
| 28 | VPCI 708/P/12 |
| 29 | VPCI 669/P/12 |
| 30 | VPCI 670/P/12 |
| 31 | VPCI 475/P/13 |
| 32 | VPCI 478/P/13 |
| 33 | VPCI 514/P/14 |
| 34 | VPCI 476/P/13 |
| 35 | VPCI 507/P/14 |
| 36 | VPCI 1130/P/13 |
| 37 | VPCI 245/P/14 |
| 38 | VPCI 247/P/14 |
| 39 | VPCI 542/P/14 |
| 40 | VPCI 250/P/14 |
| 41 | VPCI 556/P/14 |
| 42 | VPCI 557/P/14 |
| 43 | VPCI 1048/P/14 |
| 44 | VPCI 260/P/14 |
| 45 | VPCI 550/P/14 |
| 46 | VPCI 509/P/14 |
| 47 | VPCI 513/P/14 |
| 48 | VPCI 478/P/14 |
| 49 | VPCI 671/P/12 |
| 50 | VPCI 673/P/12 |
| 51 | VPCI 463/P/14 |
| 52 | VPCI 266/P/14 |
| 53 | VPCI 711/P/12 |
| 54 | VPCI 264/P/14 |
| 55 | VPCI 265/P/14 |
| 56 | VPCI 472/P/13 |
| 57 | VPCI 106/P/14 |
| 58 | VPCI 263/P/14 |
| 59 | VPCI 712/P/12 |
| 60 | VPCI 477/P/13 |
| 61 | VPCI 479/P/13 |
| 62 | VPCI 548/P/14 |
| 63 | VPCI 508/P/14 |
| 64 | VPCI 481/P/13 |
| 65 | VPCI 484/P/13 |
| 66 | VPCI 718/P/14 |
| 67 | VPCI 714/P/14 |
| 68 | VPCI 248/P/14 |
| 69 | VPCI 536/P/14 |
| 70 | VPCI 528/P/14 |
| 71 | VPCI 511/P/14 |
| 72 | VPCI 510/P/14 |
| 73 | VPCI 554/P/14 |
| 74 | VPCI 546/P/14 |
| 75 | VPCI 1133/P/13 |
| 76 | VPCI 467/P/14 |
| 77 | VPCI 473/P/13 |
| 78 | VPCI 470/P/14 |
| 79 | VPCI 674/P/12 |
| 80 | VPCI 270/P/14 |
| 81 | VPCI 474/P/13 |
| 82 | VPCI 474/P/14 |
| 83 | VPCI 459/P/14 |
| 84 | VPCI 469/P/14 |
| 85 | VPCI 482/P/13 |
| 86 | VPCI 1059/P/14 |
| 87 | VPCI 473/P/14 |
| 88 | VPCI 692/P/12 |
| 89 | VPCI 683/P/12 |
| 90 | VPCI 105/P/14 |
| 91 | KCTC 17810 |
| 92 | JCM 15448 |
| 93 | KCTC 17809 |

In some instances, the fungal infection is caused by a fungus in the genus Aspergillus (i.e., an Aspergillus infection). For example, an Aspergillus infection can be caused by a fungus in the genus Aspergillus that is selected from the group consisting of Aspergillus fumigatus, A. flavus, A. terreus, A. niger, A. candidus, A. clavatus, and A. ochraceus. Examples of Aspergillus infections that can be treated by the methods of the invention include, but are not limited to, aspergillosis (e.g., invasive aspergillosis, central nervous system aspergillosis, or pulmonary aspergillosis). In some instances, a fungal infection may also be a dermatophyte infection, which can be caused by a fungus in the genus Microsporum, Epidermophyton, or Trichophyton.

In some instances, the methods of the invention can be used to treat a Pneumocystis infection, referring to an infection caused by a fungus in the genus Pneumocystis. Fungi in the genus Pneumocystis include P. carnii, P. jirovecii, P. murina, P. oryctolagi, and P. wakefieldiae. Examples of Pneumocystis infections that can be treated by the methods of the invention include, but are not limited to Pneumocystis jirovecii pneumonia (also called Pneumocystis carnii pneumonia, Pneumocystis pneumonia, or PCP).

Further, the methods provided herein can be used to treat, for example, tinea capitis, tinea corporis, tinea pedis, onychomycosis, perionychomycosis, Pityriasis versicolor, oral thrush, vaginal candidosis, respiratory tract candidosis, biliary candidosis, eosophageal candidosis, urinary tract candidosis, systemic candidosis, mucocutaneous candidosis, aspergillosis, mucormycosis, paracoccidioidomycosis, North American blastomycosis, histoplasmosis, coccidioidomycosis, sporotrichosis, fungal sinusitis, and chronic sinusitis. Alternatively, the treatment regimens and pharmaceutical compositions described herein can be administered to treat a blood stream infection or organ infection (e.g., lung, kidney, or lover infections) in a subject.

In some instances, a fungal infection can be a drug-resistant fungal infection, which is a fungal infection that is refractory to treatment with an antifungal drug. In such infections, the fungus that causes the infection is resistant to treatment with one or more antifungal drugs (e.g., an antifungal drug-resistant strain of Candida spp.). Antifungal drugs to which the fungus may be resistant include, but are not limited to, azole compounds, echinocandins, polyene compounds, and flucytosine.

### II. Treatment Indications

The methods of treatment provided herein can be used with (i) a human subject who has one or more indications, symptoms, or signs of a fungal infection or related conditions thereto or (ii) a human subject at high risk of developing a fungal infection or related conditions thereof (e.g., hospital inpatients, intestinal transplant recipients, low birth weight infants, individuals with a genetic susceptibility). The methods disclosed herein may be used alone or in a multi-part treatment plan to treat, mitigate, or prevent conditions, diseases, or symptoms associated with fungal infections or related conditions in a human subject in need or at risk thereof.

In some instances, an individual who has developed or is at risk of developing a fungal infection can be identified based on standard diagnostic assessments. Such assessments may include testing any appropriate biological sample obtained from the individual in which signs or indicators of the fungal infection are detectable including biological fluid, stool samples, tissue, or cells (e.g., bodily fluids such as blood and blood constituents (e.g., serum and plasma), bronchial lavage sputum, saliva, urine, amniotic fluid, lymph fluid, bile, exudate, peritoneal fluid, cerebrospinal fluid, supernatant from cell lysates, lysed cells, cellular extracts, and nuclear extracts; tissue including tissue from a fresh, frozen and/or preserved organ, tissue sample, biopsy, and/or aspirate). Indicators of infection may be of host origin (e.g., cytokines or antibodies) or microbial origin (e.g., pathogen associated molecules or fungal cells as detected by fecal CFUs, mucosal biopsies, or 16S sequencing).

Indications of a fungal infection may be directly or indirectly detected in or on the body of the subject. For example, direct signs of an infection include, but are not limited to, detection of overgrowth of an undesired fungal pathobiont or pathogen (e.g., species of Candida) as measured by adherent microbial species on a tissue biopsy or a non-adherent species in other biological sample extractions (e.g., biological fluid, stool samples, tissue, or cells (e.g., bodily fluids such as blood and blood constituents (e.g., serum and plasma), bronchial lavage sputum, saliva, urine, amniotic fluid, lymph fluid, bile, exudate, peritoneal fluid, cerebrospinal fluid, supernatant from cell lysates, lysed cells, cellular extracts, and nuclear extracts; tissue including tissue from a fresh, frozen and/or preserved organ, tissue sample, biopsy, and/or aspirate), wherein the microbe is detected using methods well known in the art (e.g., culture-based methods or non-culture methods). A fungal infection may be indicated by changes (e.g., an increase or decrease) in the abundance of certain fungal taxa, genera (e.g., Candida spp.) or species (e.g., Candida tropicalis, Candida albicans) compared to a healthy individual. Alternatively, a fungal infection may be indicated by a change (e.g., increase or decrease) in the overall abundance or diversity (e.g., composition) of the total population of fungal species compared to a healthy individual.

### III. Pharmaceutical Formulations

The invention features methods for treating or preventing a fungal infection or an associated condition thereto by administering a single dose of a pharmaceutical composition including or consisting of CD101 in salt or neutral form (e.g., by oral, subcutaneous, or intravenous administration). The pharmaceutical composition may be administered to humans with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed. The amount of active component (e.g., CD101 in salt or neutral form) included in the single dose of the invention are such that a suitable dose within the designated range is provided (e.g., a dose of 50 to 800 mg or 500 mg to 1200 mg of CD101 in salt or neutral form).

A pharmaceutical composition consisting of or including CD101 in salt or neutral form may be formulated for e.g., oral administration, intravenous administration, or subcutaneous administration. In some instances, a pharmaceutical composition consisting of or including CD101 in salt or neutral form may be formulated for oral administration. In some instances, a pharmaceutical composition consisting of or including CD101 in salt or neutral form may be formulated for subcutaneous administration. In some instances, a pharmaceutical composition consisting of or including CD101 in salt or neutral form may be formulated for intravenous administration (e.g., injection or infusion). For injectable formulations, various effective pharmaceutical carriers are known in the art (See, e.g., Remington: The Science and Practice of Pharmacy, 22^{nd} ed., (2012) and ASHP Handbook on Injectable Drugs, 18^{th} ed., (2014)).

Acceptable carriers and excipients in the pharmaceutical composition of the present invention are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The compositions may be formulated according to conventional pharmaceutical practice. The concentration of the compound in the formulation will vary depending upon a number of factors, including the dosage of the drug to be administered, and the route of administration.

### Oral Formulations

The pharmaceutical composition of the present invention (e.g., CD101 in salt or neutral form) can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium).

The pharmaceutical compositions of the invention can alternatively be formulated with excipients that improve the oral bioavailability of the compound. For example, the dosage forms of the invention can be formulated for oral administration with medium chain (C8 to C12) fatty acids (or a pharmaceutically acceptable salt thereof), such as capric acid, caprylic acid, lauric acid, or a pharmaceutically acceptable salt thereof, or a mixture thereof. The formulation can optionally include a medium chain (C8 to C12) alkyl alcohol, among other excipients. Alternatively, the compounds of the invention can be formulated for oral administration with one or more medium chain alkyl saccharides (e.g., alkyl (C8 to C14) beta-D-maltosides, alkyl (C8 to C14) beta-D-Gulcosides, octyl beta-D-maltoside, octyl beta-D-maltopyranoside, decyl beta-D-maltoside, tetradecyl beta-D-maltoside, octyl beta-D-glucoside, octyl beta-D-glucopyranoside, decyl beta-D-glucoside, dodecyl beta-D-glucoside, tetradecyl beta-D-glucoside) and/or medium chain sugar esters (e.g., sucrose monocaprate, sucrose monocaprylate, sucrose monolaurate and sucrose monotetradecanoate).

The methods disclosed herein may also further include the administration of an immediate-release, extended release or delayed-release formulation of CD101 in salt or neutral form.

### Parenteral Formulations

The pharmaceutical composition of the present invention (e.g., CD101 in salt or neutral form) may be formulated in the form of liquid solutions or suspensions and administered by a parenteral route (e.g., subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), F-12 medium). Formulation methods are known in the art, see e.g., Gibson (ed.) Pharmaceutical Preformulation and Formulation (2nd ed.) Taylor & Francis Group, CRC Press (2009).

### IV. Dosage and Administration

The methods described herein provide a treatment for a fungal infection by administering a single dose of a pharmaceutical composition including CD101 (e.g., CD101 in salt or neutral form), wherein the single dose is administered in an amount sufficient to treat the fungal infection without requiring additional doses of an antifungal agent. In some instances, the single dose of CD101 (e.g., CD101 in salt or neutral form) is administered without concurrent administration of an additional antifungal agent (e.g., CD101 or another antifungal agent) within a time period (e.g., within 1 min, 30 min, 1 hr, 2 hr, 12 hr, 24 hr, 2 days, 3 days, 4 days, 5 days, 6, days, 1 week before or after administration of the single dose of CD101) that would confer therapeutic benefits (e.g., be systemically active) at the same time that CD101 is at a therapeutically effective concentration in the subject. In some instances, the single dose treatment is not combined with any other antifungal treatment within 1-21 days before or after administration. For example, a single dose of CD101 may be administered (e.g., orally, intravenously, subcutaneously, or intramuscularly) to a subject with a fungal infection and the single dose effectively treats the fungal infection without necessitating additional antifungal treatments before, during, or after the single dose treatment with CD101. In some instances, the individual may have undergone previous but unsuccessful attempts of treatment with a different antifungal agent (e.g., an antifungal agent that does not confer therapeutic benefit) at any time prior to the single dose treatment (e.g., in cases of antifungal-resistant fungal infections) and treatment with a single dose formulation including CD101 (e.g., CD101 in salt or neutral form) is sufficient and effective to treat the infection.

The dose of CD101 (e.g. CD101 in salt or neutral form) in the present invention depends on factors including the route of administration, the disease to be treated, and physical characteristics, e.g., age, weight, general health, of the human subject). The dose may be adapted by the physician in accordance with conventional factors such as the extent of the disease and different parameters of the subject (e.g., human). Typically, the amount of CD101 (e.g. CD101 in salt or neutral form) contained within one or more doses may be an amount that effectively reduces the risk of or treats a fungal infection and associated conditions in a human subject without inducing significant toxicity.

The pharmaceutical compositions of the invention can be administered to human subjects in therapeutically effective amounts. The preferred dosage of drug to be administered is likely to depend on such variables as the type and extent of the disorder, the overall health status of the particular human subject, the specific compound being administered, the excipients used to formulate the compound, and its route of administration.

In some instances, the single dose can include an oral formulation of CD101 (e.g. CD101 in salt or neutral form), and can be administered in doses of about 50 mg to about 1200 mg (e.g., 75±25 mg, 100±25 mg, 150±50 mg, 200±50 mg, 250±50 mg, 300±50 mg, 350±50 mg, 400±50 mg, 500±100 mg, 600±100 mg, 700±100 mg, 1800±100 mg, 1900±50 mg, 1000 mg±500 mg, or 1500±500 mg). In other instances, the single dose of CD101 (e.g. CD101 in salt or neutral form) can include a parenteral formulation (e.g., intravenous, subcutaneous, or intramuscular), and can be administered in dosages of about 50-1200 mg (e.g., 75±25 mg, 100±25 mg, 150±50 mg, 200±50 mg, 250±50 mg, 300±50 mg, 350±50 mg, 400±50 mg, 450±50 mg, 500±100 mg, 600±100 mg, 700±100 mg, 800±100 mg, 900±50 mg, 1000 mg±100 mg, or 1100±100 mg).

In any of the methods described herein, the timing of the administration of the single dose treatment with CD101 (e.g., CD101 in salt or neutral form) depends on the medical and health status of the human subject. In some instances, the human subject is at risk for developing a fungal infection or a related condition and receives the single dose treatment with CD101 (e.g., CD101 in salt or neutral form) before developing symptoms or signs of a fungal infection. In some instances, the human subject has already developed a fungal infection or a related condition and receives the single dose treatment with CD101 (e.g., CD101 in salt or neutral form). The timing of the administration of the single dose of CD101 (e.g., CD101 in salt or neutral form) may be optimized by a physician to reduce the risk of or to treat a fungal infection in a human subject.

### EXAMPLES

### Example 1: Subcutaneous Injection of CD101

Single dose subcutaneous (SC) administration may further extend the utility of CD101 beyond that of other echinocandins, to antifungal treatment and prophylaxis in the outpatient setting. Preclinical studies were conducted to evaluate the feasibility of using SC administration of CD101 for these purposes.

### Methods

The efficacy of CD101 SC was studied in an immunocompetent DBA/2 mouse model of disseminated candidiasis. Mice (5/grp) were challenged with Candida albicans SC5314 (ATCC: MYA-2876, fluconazole-sensitive human clinical isolate shown to be pathogenic in mice) via IV injection (100 μL, 5.0 log CFU/mouse) and treated with CD101 SC (1, 3 or 10 mg/kg). Micafungin via IP administration was tested as a positive control at the same three doses. At 24 hours following challenge, kidneys were harvested and processed for CFU enumeration. All comparisons were made between the treatment and time-matched vehicle groups. CD101 SC (5 mg/kg) was also tested in a similar disseminated candidiasis model using ICR mice rendered neutropenic by cyclophosphamide on Day −4 (150 mg/kg) and Day −1 (100 mg/kg) prior to infection by the same C. albicans SC5314 strain (IV injection, 100 μL, 4.5 log CFU/mouse, See Example 10).

Previous toxicology studies by the IV route of administration conducted in cynomolgus monkeys have shown CD101 to be safe and well tolerated at up to at least 30 mg/kg, which generates very high systemic exposures upon initial infusion of CD101 into the bloodstream. Therefore, only local tolerability (and PK) of CD101 by SC administration required evaluation. For this purpose, male and female monkeys were observed for up to 10 days following a single 30 mg/kg SC dose. In the same study, to determine the pharmacokinetics of CD101 following SC administration, whole blood samples were collected and the plasma was harvested at approximately 0.25, 0.5, 1, 2, 4, 8, 24, 36, and 48 hours, and 3, 4, 5, 7, and 10 days postdose. Plasma concentrations were then quantified by liquid chromatography with tandem mass spectrometric detection (LC-MS/MS). Bioavailability from SC dosing was calculated by comparing the calculated area under the concentration-time profile (AUC) from SC against the AUC from IV administration of the same dose.

### Results

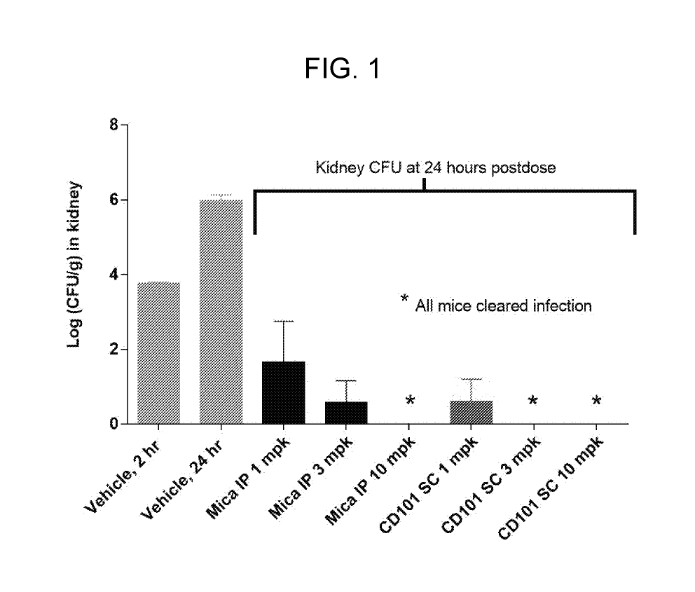

In the DBA/2 mouse efficacy study (FIG. 1), at 2 hr post infection, vehicle-treated mice demonstrated an average kidney CFU of 3.8 log CFU that increased to 6.1 log CFU at 24 hr. Groups treated with CD101 SC (1, 3, and 10 mg/kg) showed significant reduction in kidney CFU when compared with the vehicle control. Animals receiving 3 or 10 mg/kg of CD101 SC showed complete CFU clearance, and 4 of 5 animals in the 1 mg/kg group were completely cleared of CFU burden by 24 hr. Micafungin also showed good dose response in CFU reduction with the same treatment doses but complete clearance was only observed with the 10 mg/kg dose suggesting that it was less efficacious than CD101 at comparable doses.

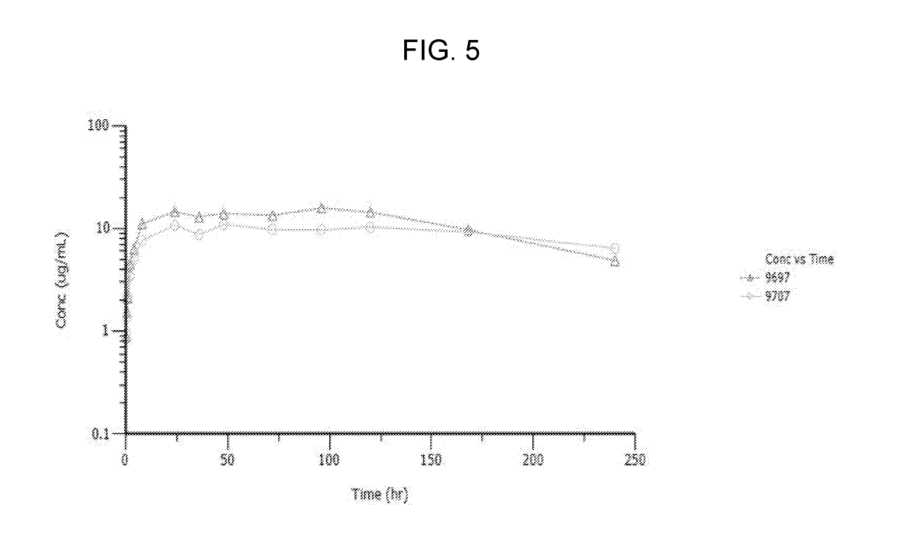

Early pharmacokinetic studies in rats and monkeys had indicated that CD101 subcutaneous administration was well-tolerated, although these initial studies were aimed at characterizing the pharmacokinetics at lower doses 5 mg/kg). Therefore, a separate study was designed to evaluate the tolerability of a SC dose of CD101 as a highly concentrated solution (100 mg/mL; 30 mg/kg) in two cynomolgus monkeys. FIG. 5 shows plasma levels of CD101 in two cynomolgus monkeys over 10 days after a single 30 mg/kg dose administered subcutaneously. The drug reaches a maximum concentration within a few hours then remains nearly constant over the course of 10 days. Table 2 further shows various pharmacokinetic characteristics of SC administered CD101 in the monkeys. Table 3 shows the SC formulation of CD101 used in the monkey study.

TABLE 2

| | | | | | AUClast | | Half- |
|---|---|---|---|---|---|---|---|
| | | Dose | Tmax | Cmax | (hr*μg/ | AUCINF_obs | life |
| Animal | Sex | (mg/kg) | (hr) | (μg/mL) | mL) | (hr*μg/mL) | (hr) |
| 9697 | M | 30 | 96 | 15.9 | 2740 | 3280 | 76.2 |
| 9707 | F | 30 | 48 | 10.9 | 2180 | 3760 | 171 |

TABLE 3

| Component | Function | Concentration |
|---|---|---|
| CD101 acetate | Active ingredient | 100 mg/mL |
| Mannitol | Tonicity | 11.4 mg/mL |
| HCI | pH adjustment | As needed to adjust to pH 5.6 |
| NaOH | pH adjustment | As needed to adjust to pH 5.6 |
| Water for injection | medium | q.s to 1.0 mL |

In the monkey SC tolerability/PK study, no sign of irritation or local (injection site) adverse toxicity was noted following a single high dose of 30 mg/kg CD101. There was also no effect on bodyweight or food consumption upon further follow-up observations for 10 days after administration.

From the same monkey SC tolerability/PK study, the pharmacokinetic profile following SC administration of CD101 at 30 mg/kg showed that total exposure measured over a 10-day period was comparable (80% bioavailability) to that following IV administration at the same dose (FIG. 2), indicating high/equivalent bioavailability from SC administration. The maximum plasma concentration from SC administration was reached after 24 hours and was sustained throughout the first week post-dose. Concentrations started to decrease one week after injection and the terminal half-life estimated was high at approximately 124 hours. Table 4 further shows various pharmacokinetic characteristics of subcutaneously and intravenously administered CD101.

TABLE 4

| | | | | AUCₗₐₛₜ | AUC_{inf} | Half- |
|---|---|---|---|---|---|---|
| | Dose | Tₘₐₓ | Cₘₐₓ | (μg*hr/ | (μg*hr/ | life |
| Route | (mg/kg) | (hr) | (μg/mL) | mL) | mL) | (hr) |
| Subcutaneous | 30 | 72 | 13.4 | 2460 | 3520 | 124 |
| IV | 30 | 1 | 112 | 3135 | 3340 | 49.7 |

### Example 2: Treatment of a Fungal Infection in a Human Subject with Candidemia by a Single Intravenous Dose of CD101

A human subject is diagnosed with candidemia using standard diagnostic procedures. The subject receives treatment with a single dose of 50-800 mg (e.g., 75±25 mg, 100±25 mg, 150±50 mg, 200±50 mg, 250±50 mg, 300±50 mg, 350±50 mg, 400±50 mg, 450±50 mg, 500±100 mg, 600±100 mg, 700±100 mg, 800±100 mg, 900±50 mg, 1000 mg±100 mg, or 1100±100 mg) of the acetate salt of CD101 administered by intravenous infusion. No other antifungal treatment is provided to the subject within one to three weeks before or after administration of the single dose CD101 treatment. Following the single intravenous administration of CD101 (e.g., one to three weeks later), the subject is assessed in a follow-up visit and the candidemia infection is confirmed to be resolved.

### Example 3: Treatment of a Fungal Infection in a Human Subject with Invasive Candidiasis by a Single Subcutaneous Dose of CD101

A human subject is diagnosed with invasive candidiasis using standard diagnostic procedures. The subject receives treatment with a single dose of 50-1200 mg (e.g., 75±25 mg, 100±25 mg, 150±50 mg, 200±50 mg, 250±50 mg, 300±50 mg, 350±50 mg, 400±50 mg, 450±50 mg, 500±100 mg, 600±100 mg, 700±100 mg, 800±100 mg, 900±50 mg, 1000 mg±100 mg, or 1100±100 mg) of the acetate salt of CD101 administered by subcutaneous injection. No other antifungal treatment is provided to the subject within one to three weeks before or after administration of the single dose CD101 treatment. Following the single subcutaneous administration of CD101 (e.g., one to three weeks later), the subject is assessed in a follow-up visit and the invasive candidiasis infection is confirmed to be resolved.

### Example 4: Treatment of a Fungal Infection in a Human Subject with Aspergillosis by a Single Oral Dose of CD101

A human subject is diagnosed with aspergillosis using standard diagnostic procedures. The subject receives treatment with a single dose of 50 mg-1200 mg (e.g., 75±25 mg, 100±25 mg, 150±50 mg, 200±50 mg, 250±50 mg, 300±50 mg, 350±50 mg, 400±50 mg, 450±50 mg, 500±100 mg, 600±100 mg, 700±100 mg, 800±100 mg, 900±50 mg, 1000 mg±100 mg, or 1100±100 mg) of the acetate salt of CD101 administered in an oral formulation (e.g., in a pill, capsule, or liquid formulation). No other antifungal treatment is provided to the subject within one to three weeks before or after administration of the single dose CD101 treatment. Following the single oral administration of CD101 (e.g., one to three weeks later), the subject is assessed in a follow-up visit and the aspergillosis infection is confirmed to be resolved.

### Example 5: Efficacy of CD101 in Mouse Models of Aspergillosis and Azole-Resistant Disseminated Candidiasis

### Methods

The in vivo efficacy of CD101 was evaluated using neutropenic mouse models of azole-resistant candidiasis and aspergillosis. An azole-resistant strain of Candida albicans (R357; resistant to fluconazole [Flu], voriconazole, and posaconazole but susceptible to amphotericin B [AmB] and echinocandins) isolated from human blood was used for the mouse candidiasis model. A test strain of Aspergillus fumigatus (ATCC 13073) was used for the mouse aspergillosis model. Mice were rendered neutropenic by cyclophosphamide and then infected by injections of C. albicans (10⁵ CFU/mouse) or A. fumigatus (10⁴ CFU/mouse) into the tail vein. Test articles were administered starting 2 hours after infection. In the mouse candidiasis model, groups of 5 mice each received one dose of AmB (3 mg/kg IV), Flu (20 mg/kg orally), or CD101 (3, 10 or 30 mg/kg by intraperitoneal administration [IP]). After 72 hours post-infection, mice were euthanized and C. albicans counts in kidney tissue (CFU/g) were measured. In the mouse aspergillosis model, groups of 10 mice each received one dose of AmB (2 mg/kg IP) or CD101 (2 mg/kg IV and IP). Survival was monitored daily for 10 days. Differences between vehicle and test article groups were assessed for significance by one-way ANOVA followed by Dunnett's test and Fisher's Exact test in the candidiasis and aspergillosis models, respectively.

### Results

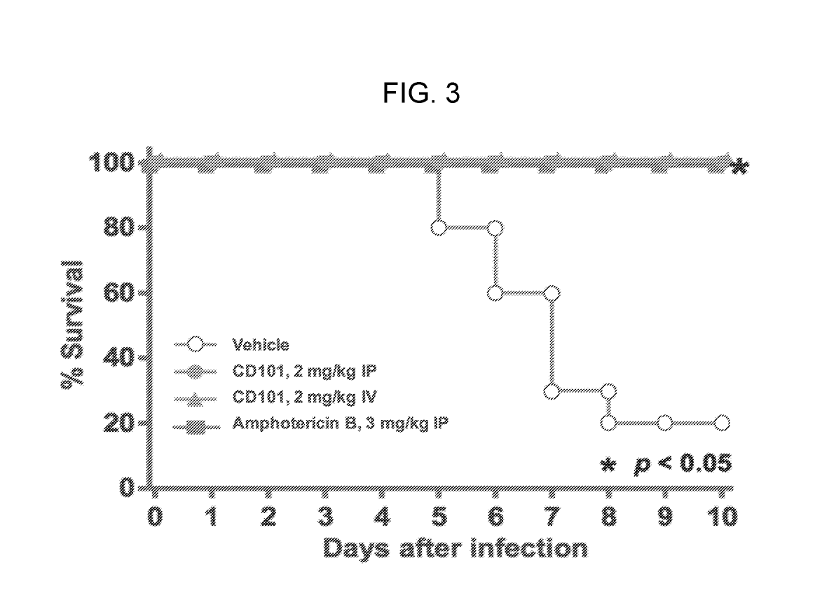

One dose of CD101 3 mg/kg produced a >99.9% (or >3-log; P<0.001) reduction in C. albicans CFU compared with vehicle through at least 72 hours post-dose following a single IP dose. AmB showed similar, albeit less robust, efficacy (>99% or >2-log reduction in CFU; P<0.05), whereas fluconazole was less efficacious (83.9% or <2-log reduction in CFU). In the aspergillosis model, CD101 administered 2 mg/kg IV or IP showed similar efficacy to that of AmB 2 mg/kg IP, both with significantly longer survival than vehicle (P<0.05; FIG. 3).

### Conclusions

A single dose of CD101 3 mg/kg produced significant reduction in C. albicans burden compared with vehicle (P<0.001) in the neutropenic mouse model of azole-resistant candidiasis, demonstrating efficacy comparable, if not better, to that of AmB at the same dose.

### Example 6: Efficacy of CD101 Against Candida auris Clinical Isolates

### Materials and Methods

Organisms and Antifungal Agents

C. auris clinical isolates obtained from Japan, South Korea, India and the Center for Medical Mycology (n=14) were evaluated. The following Candida QC strains approved for yeast and moulds by the Clinical and Laboratory Standards Institute (CLSI, Document M38-A2 and M27-A3) were used: C. parapsilosis ATCC 22019, C. krusei ATCC 6258. Test compounds were prepared fresh prior to use in MIC assays and included: CD101, 5-flucytosine (5FC), amphotericin B (AMB), anidulafungin (ANID), caspofungin (CAS), fluconazole (FLU), itraconazole (ITRA), micafungin (MICA), posaconazole (POSA) and voriconazole (VORI).

Minimum Inhibitory Concentration (MIC) Assays

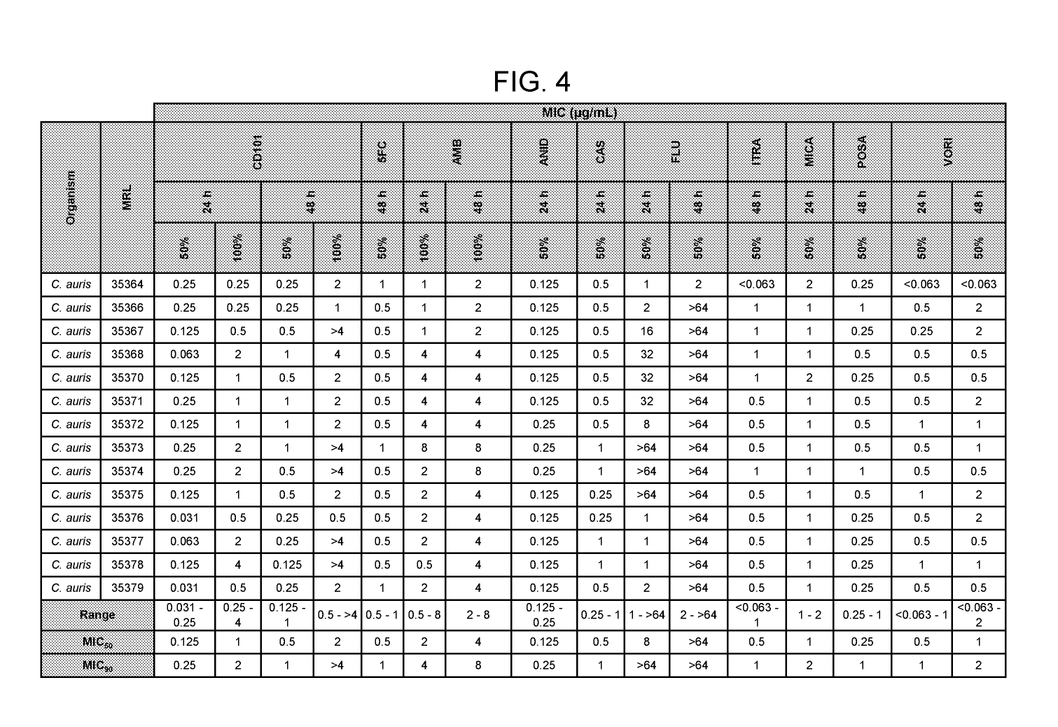

Broth microdilution MIC assays performed according to CLSI M38-A2 and M27-A3 methodology were used to evaluate the susceptibility of the fungal strains to the selected antifungals. Briefly, C. auris strains were plated on Sabouraud Dextrose Agar (SDA) and incubated at 37° C. for 2 days. C. auris cells were then harvested then washed in normal saline (0.85% NaCl via centrifugation). MIC assay inoculums were prepared using a hemocytometer. MIC assays were read after 24 and/or 48 hours incubation at 50 and/or 100% inhibition (FIG. 4). To check the inoculum count, ten-fold dilutions of C. auris working conidial suspension were plated onto SDA media. Inoculum plates were incubated at 37° C. for 2 days prior to determining colony count.

### Example 7: Efficacy CD101, Caspofungin (CAS), Micafungin (MICA), and Fluconazole (FLU) Against Candida auris Clinical Isolates and FKS1 HS1 Sequence Analysis

This study was to determine in vitro susceptibility of clinical C. auris isolates to CD101, caspofungin (CAS), micafungin (MICA), and fluconazole (FLU), and to analyze the sequence of hot spot 1 (HS1) within FKS1.

### Materials and Methods

Candida auris isolates. Thirty-eight C. auris strains, obtained from VP Chest Institute, University of Delhi (Delhi, India) were used in the study (Table 5). Strains were grown on yeast extract peptone dextrose (YPD) agar plates prior to testing.

TABLE 5

| Strain # | C. auris strain # (India) |
|---|---|
| 1 | VPCI 669/P/12 |
| 2 | VCPI 671/P/12 |
| 3 | VCPI 674/P/12 |
| 4 | VCPI 683/P/12 |
| 5 | VCPI 692/P/12 |
| 6 | VCPI 712/P/12 |
| 7 | VCPI 471/P/13 |
| 8 | VCPI 475/P/13 |
| 9 | VCPI 478/P/13 |
| 10 | VCPI 479/P/13 |
| 11 | VCPI 480/P/13 |
| 12 | VCPI 482/P/13 |
| 13 | VCPI 483/P/13 |
| 14 | VCPI 1130/P/13 |
| 15 | VCPI 1132/P/13 |
| 16 | VCPI 1133/P/13 |
| 17 | VCPI 105/P/14 |
| 18 | VCPI 107/P/14 |
| 19 | VCPI 510/P/14 |
| 20 | VCPI 511/P/14 |
| 21 | VCPI 512/P/14 |
| 22 | VCPI 513/P/14 |
| 23 | VCPI 514/P/14 |
| 24 | VCPI 250/P/14 |
| 25 | VCPI 265/P/14 |
| 26 | VCPI 266/P/14 |
| 27 | VCPI 462/P/14 |
| 28 | VCPI 463/P/14 |
| 29 | VCPI 467/P/14 |
| 30 | VCPI 471a/P/14 |
| 31 | VCPI 478/P/14 |
| 32 | VCPI 518/P/14 |
| 33 | VCPI 550/P/14 |
| 34 | VCPI 714/P/14 |
| 35 | VCPI 717/P/14 |
| 36 | VCPI 1060/P/14 |
| 37 | VCPI 74/P/15 |
| 38 | VCPI 213/P/15 |

Candida auris antifungal susceptibility testing (AFST) Antifungal susceptibility testing was performed in duplicate for each strain in accordance with the guidelines described in CLSI documents M27-A3 (CLSI, 2008). C. parapsilosis ATCC 22019 and C. krusei ATCC 6258 were used as quality control strains. CD101, CAS, MICA, and FLU were obtained as standard powders from their manufacturer, and stock solutions were prepared by dissolving the compounds in water (CAS, MICA) or 100% dimethyl sulfoxide (DMSO) (CD101, FLU).

FKS1 HS1 PCR/sequencing. FKS1 HS1 PCR was carried out in the T100 thermal cycler (Bio-Rad) in a 30-μl reaction volume using EmeraldAmp MAX PCR Master Mix (TaKaRa). PCR mixtures contained 1 μl of each primer: Cspp_F2275 (5′-AATGGGCTGGTGCTCAACAT-3′) and Cspp_R3070 (5′-CCTTCAATTTCAGATGGAACTTGATG-3′) at 10 μM. A sterile toothpick with a touch of testing single colony was dipped into the PCR reaction mastermix, and then FKS1 HS1 PCR were performed. The time-temperature profile included initial denaturation for 3 min at 94° C. followed by 35 cycles of 30 s at 94° C., 30 s at 53° C., and 90 s at 72° C. Amplicons were visualized on GelStar Nucleic Acid Gel Stain (Lonza) stained 1% agarose gel, purified by using ZR DNA Sequencing Clean-up Kit (Zymo Research), and sequenced by Genewiz. Sequencing results were analyzed by SeqMan Pro 14 (DNASTAR Lasergene).

### Results

Candida auris Antifungal Susceptibility Testing (AFST).

The MIC (μg/ML) distributions of C. auris isolates for CD101, CAS, MICA, and FLU are shown in Table 6. All C. auris isolates (38) were resistant to fluconazole. Four (4) isolates were resistant to all tested echinocandins (CD101, CAS, MICA). CD101 exhibited activity similar to MICA.

FKS1 HS1 PCR/sequencing. Results of C. auris isolates FKS1 HS1 sequence analysis are shown in Table 6. Thirty four (34) echinocandin-sensitive isolates presented wild-type (WT) genotype within FKS1 HS1 region. Four (4) isolates (strain #s: 16, 25, 27, and 30 in Table 6), demonstrating reduced susceptibility to echinocandins, exhibited serine to phenylalanine amino acid substitution in position equivalent to FKS1 HS1 S645 in Candida albicans.

TABLE 6
In vitro antifungal susceptibility profile and FKS1 HS1 characteristics of Candida auris strains

| | C. auris | | | | | | | | | |
|---|---|---|---|---|---|---|---|---|---|---|
| | strain # | CD101 | | CAS | | MICA | | FLU | | |
| Strain # | (India) | 24 h | 48 h | 24 h | 48 h | 24 h | 48 h | 24 h | 48 h | FKS1 HS1 |
| 1 | VPCI | 0.5 | 0.5 | 0.25* | >16** | 0.125 | 0.25 | >128 | >128 | WT |
| | 669/P/12 | | | | | | | | | |
| 2 | VCPI | 0.5 | 0.5 | 0.25* | >16** | 0.125 | 0.25 | >128 | >128 | WT |
| | 671/P/12 | | | | | | | | | |
| 3 | VCPI | 0.25 | 0.25 | 0.25* | 0.25* | 0.06 | 0.125 | >128 | 128 | WT |
| | 674/P/12 | | | | | | | | | |
| 4 | VCPI | 0.5 | 0.5 | 0.25* | >16** | 0.125 | 0.25 | >128 | >128 | WT |
| | 683/P/12 | | | | | | | | | |
| 5 | VCPI | 0.5 | 0.5 | 0.25* | >16** | 0.125 | 0.25 | >128 | >128 | WT |
| | 692/P/12 | | | | | | | | | |
| 6 | VCPI | 0.25 | 0.25 | 0.25* | 0.25* | 0.125 | 0.125 | >128 | 128 | WT |
| | 712/P/12 | | | | | | | | | |
| 7 | VCPI | 0.5 | 0.5 | 1* | >16** | 0.25 | 0.25 | >128 | >128 | WT |
| | 471/P/13 | | | | | | | | | |
| 8 | VCPI | 0.5 | 0.5 | 0.25* | 0.25* | 0.125 | 0.25 | >128 | 64 | WT |
| | 475/P/13 | | | | | | | | | |
| 9 | VCPI | 0.25 | 0.25 | 0.25* | >16** | 0.125 | 0.125 | >128 | 128 | WT |
| | 478/P/13 | | | | | | | | | |
| 10 | VCPI | 0.25 | 0.25 | 0.5* | >16** | 0.125 | 0.125 | >128 | 128 | WT |
| | 479/P/13 | | | | | | | | | |
| 11 | VCPI | 0.25 | 0.25 | 0.5* | >16** | 0.125 | 0.125 | >128 | >128 | WT |
| | 480/P/13 | | | | | | | | | |
| 12 | VCPI | 0.25 | 0.25 | 0.5* | >16** | 0.125 | 0.125 | >128 | >128 | WT |
| | 482/P/13 | | | | | | | | | |
| 13 | VCPI | 0.25 | 0.25 | 0.25* | 0.25* | 0.125 | 0.125 | >128 | 128 | WT |
| | 483/P/13 | | | | | | | | | |
| 14 | VCPI | 0.5 | 0.25 | 0.5* | 0.5* | 0.125 | 0.125 | 32 | 64 | WT |
| | 1130/P/13 | | | | | | | | | |
| 15 | VCPI | 0.5 | 0.25 | 0.5* | 0.5* | 0.125 | 0.125 | 128 | 128 | WT |
| | 1132/P/13 | | | | | | | | | |
| 16 | VCPI | 16 | >16 | 16 | >16 | 16 | >16 | >128 | >128 | S645F/S |
| | 1133/P/13 | | | | | | | | | |
| 17 | VCPI | 0.25 | 0.25 | 0.5* | 1* | 0.125 | 0.125 | 128 | 128 | WT |
| | 105/P/14 | | | | | | | | | |
| 18 | VCPI | 0.125 | 0.125 | 0.25* | 0.5* | 0.06 | 0.06 | 64 | >128 | WT |
| | 107/P/14 | | | | | | | | | |
| 19 | VCPI | 0.25 | 0.25 | 0.5* | >16** | 0.125 | 0.125 | >128 | >128 | WT |
| | 510/P/14 | | | | | | | | | |
| 20 | VCPI | 0.25 | 0.25 | 0.5* | >16** | 0.125 | 0.125 | 128 | >128 | WT |
| | 511/P/14 | | | | | | | | | |
| 21 | VCPI | 0.25 | 0.25 | 0.5* | 1* | 0.125 | 0.125 | 16 | >128 | WT |
| | 512/P/14 | | | | | | | | | |
| 22 | VCPI | 0.5 | 0.5 | 1* | >16** | 0.125 | 0.25 | >128 | >128 | WT |
| | 513/P/14 | | | | | | | | | |
| 23 | VCPI | 0.5 | 0.5 | 0.5* | 1* | 0.25 | 0.25 | >128 | >128 | WT |
| | 514/P/14 | | | | | | | | | |
| 24 | VCPI | 0.25 | 0.25 | 0.5 | 0.5* | 0.125 | 0.125 | 64 | >128 | WT |
| | 250/P/14 | | | | | | | | | |
| 25 | VCPI | 16 | >16 | 8 | >16 | 16 | >16 | >128 | >128 | S645F |
| | 265/P/14 | | | | | | | | | |
| 26 | VCPI | 0.5 | 0.25 | 0.125* | 0.125* | 0.125 | 0.125 | >128 | >128 | WT |
| | 266/P/14 | | | | | | | | | |
| 27 | VCPI | 16 | >16 | 8 | >16 | 16 | >16 | >128 | >128 | S645F |
| | 462/P/14 | | | | | | | | | |
| 28 | VCPI | 0.125 | 0.125 | 0.5* | 0.5* | 0.125 | 0.125 | >128 | >128 | WT |
| | 463/P/14 | | | | | | | | | |
| 29 | VCPI | 0.25 | 0.25 | 0.125* | 0.5* | 0.25 | 0.25 | >128 | >128 | WT |
| | 467/P/14 | | | | | | | | | |
| 30 | VCPI | 16 | >16 | 4 | >16 | 16 | >16 | >128 | >128 | S645F |
| | 471a/P/14 | | | | | | | | | |
| 31 | VCPI | 0.5 | 1 | 1* | 2* | 0.5 | 0.5 | >128 | >128 | WT |
| | 478/P/14 | | | | | | | | | |
| 32 | VCPI | 0.5 | 0.5 | 0.5* | 16** | 0.25 | 0.5 | >128 | >128 | WT |
| | 518/P/14 | | | | | | | | | |
| 33 | VCPI | 0.5 | 1 | 0.125* | 0.25* | 0.25 | 0.5 | >128 | >128 | WT |
| | 550/P/14 | | | | | | | | | |
| 34 | VCPI | 0.25 | 0.25 | 0.5* | >16** | 0.125 | 0.25 | >128 | >128 | WT |
| | 714/P/14 | | | | | | | | | |
| 35 | VCPI | 0.25 | 0.25 | 0.5* | >16** | 0.125 | 0.125 | 4 | >128 | WT |
| | 717/P/14 | | | | | | | | | |
| 36 | VCPI | 0.25 | 0.25 | 0.5* | >16** | 0.25 | 0.25 | >128 | >128 | WT |
| | 1060/P/14 | | | | | | | | | |
| 37 | VCPI | 0.25 | 0.25 | 0.5* | >16** | 0.25 | 0.25 | >128 | >128 | WT |
| | 74/P/15 | | | | | | | | | |
| 38 | VCPI | 0.25 | 0.25 | 0.5* | >16** | 0.25 | 0.25 | >128 | >128 | WT |
| | 213/P/15 | | | | | | | | | |

(*CAS paradoxical effect -> 16 μg/ml; **loss of CAS paradoxical effect, no possibility to read MIC, fungal growth reduction <50%)

### Conclusions

High fluconazole resistance is common in clinical isolates of C. auris. Most C. auris strains are susceptible to echinocandins. However, most strains breakthrough on caspofungin at 48 h but not with CD101 or other echinocandins. Highly reduced susceptibility to echinocandins in these C. auris isolates was associated with amino acid substitution (serine into phenylalanine, position equivalent to C. albicans S645) within the FKS1 HS1 region.

### Example 8: Efficacy CD101 in the Treatment of Candida auris Infection in a Murine Model of Disseminated Candidiasis

### Methods

Female 6-8 week old Day −1 mice were immunosuppressed with cyclophosphamide (200 mg/kg) 3 days prior to infection and 150 mg/kg 1 day post-infection. On the day of infection, mice were inoculated with 3×10⁷ C. auris blastospores via the lateral tail vein. Mice were randomized into 5 groups (n=5 for colony forming units (CFU) and n=10 for survival): CD101 20 mg/kg administered by intraperitoneal (IP) injection, fluconazole 20 mg/kg administered per os (PO), amphotericin B 0.3 mg/kg IP, and a vehicle control. Treatments were administered 2 hours post-infection (Day 1) and again on Day 4 of the study for a total of 2 doses. Mice were monitored daily and a survival curve was generated. CFU groups were sacrificed on Day 8 of the study. One kidney was removed from each mouse, homogenized, plated on potato dextrose agar (PDA), and incubated at 35° C. for 2 days to determine CFU. The remaining survival mice were monitored until the end of the study (Day 14).

### Results

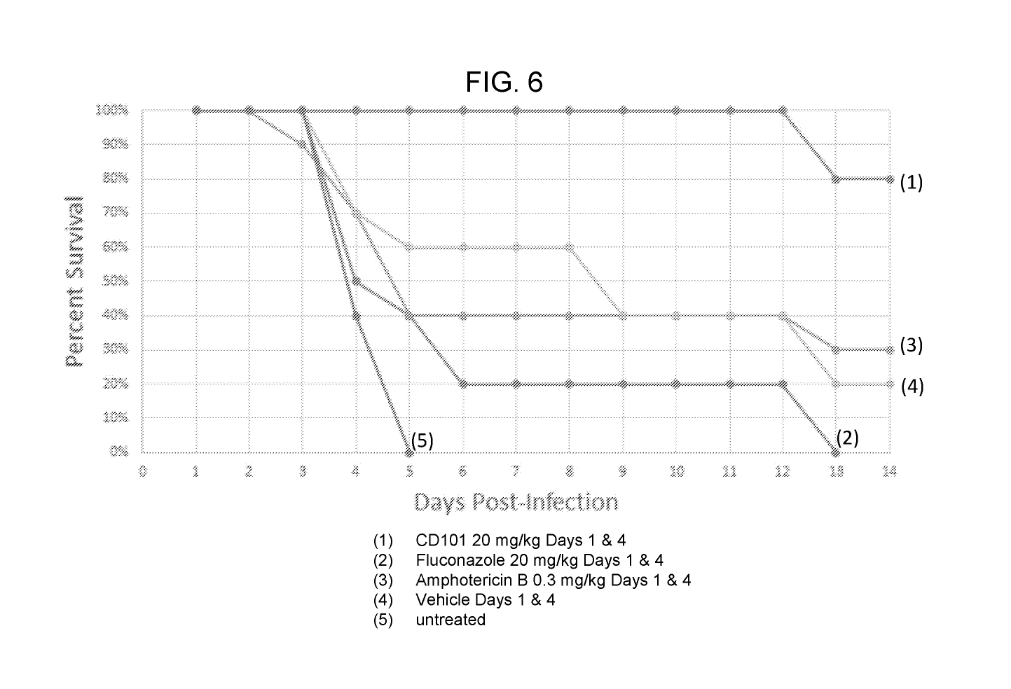

CD101 showed an average 3 log reduction in kidney CFU compared to fluconazole, amphotericin B, and vehicle treated groups, which was statistically significant (P=0.03, 0.03, and 0.04, respectively). At the end of the study, percent survival of mice in CD101, fluconazole, amphotericin B, vehicle, and untreated groups was 80, 0, 30, 20, and 0%, respectively (FIG. 6).

### Conclusion

Taken together, our findings show that CD101 possesses potent antifungal activity against C. auris infection in a disseminated model of candidiasis. Additionally, treatment with CD101 resulted in a significantly higher overall percent survival.

### Example 9: Evaluate the Ability of CD101 to Prevent and Treat Candida albicans Biofilms and Explore its Temporal Effect by Time Lapse Photography

In this study, we determined the effect of CD101 on prevention and treatment of biofilms formed by Candida albicans in vitro, and evaluated the effect of CD101 (at effective concentration) on formation of biofilm in real time using Time Lapse Microscopy (TLM).

### Materials and Methods

Test Compounds CD101 powder stocks were reconstituted in water or Yeast Nitrogen Base (YNB) medium, and diluted in YNB to a final working concentration of 0.25 μg/ml and 1 μg/ml. YNB with no CD101 was prepared in parallel and used as controls. Fluconazole was used as a comparator.

Test Media

YNB and Sabouraud dextrose agar (SDA) media

CD101 (powder and reconstituted solution) stored at −80° C. when not in use.

Strains

C. albicans SC-5314 was used for the current study.

Activity of CD101 Against Candida Biofilms

In this study, biofilms were grown in vitro using a biofilm model (Chandra et al., Nature Protocols 3:1909, 2008) and the effect of CD101 on adhesion phase biofilms (representing prevention of biofilms) or mature phase biofilms (representing treatment of biofilms) was determined.

Activity Against Adhesion Phase (Prevention) or Mature Phase (Treatment) Biofilms

Biofilms were formed on silicone elastomer (SE) discs using a catheter-associated-biofilm model (Chandra et al., Nature Protocols 3:1909, 2008; Chandra et al., J. Bacteriol. 183: 5385, 2001; Chandra et al., J. Dental Research 80: 903, 2001). For evaluation of activity against adhesion phase biofilms (prevention), Candida cells were adhered to catheter discs for 90 min. Next, discs were incubated for 24 h with CD101 (0.25 or 1 μg/ml concentrations) to allow biofilm formation. For evaluation of activity against mature phase biofilms (treatment), Candida cells were adhered to catheter discs for 90 min, then transferred to fresh media and incubated for a further 24 h to allow formation of biofilms. Mature biofilms were then exposed to CD101 (0.25 or 1 μg/ml concentrations) for another 24 h. Discs incubated with fluconazole or media alone were used as controls in all experiments.

At the end of drug exposure in both adhesion and mature phase biofilms, biofilms were quantified by measuring their metabolic activity using XTT assay (Chandra et al., Nature Protocols 3:1909, 2008; Chandra et al., J. Bacteriol. 183: 5385, 2001; Chandra et al., J. Dental Research 80: 903, 2001). Following incubation with drugs, discs were transferred to fresh plates containing phosphate buffered saline with XTT and menadione, incubated for 3 hours at 37° C. and optical density was read at 492 nm. Separate batches of biofilms were stained with fluorescent dyes (FUN1™, CONA) and observed under Confocal Scanning Laser Microscope (CSLM) to evaluate biofilm architecture and thickness (Chandra et al., Nature Protocols 3:1909, 2008; Chandra et al., J. Bacteriol. 183: 5385, 2001).

Time Lapse Microscopy

The effective CD101 concentration obtained from the above experiments was used to monitor its effect on biofilm formation in real time using TLM, which involves capturing real-time images of a single frame at specific time intervals, allowing temporal monitoring of the interactions occurring between the drug and Candida biofilms. Captured images were combined in a time sequence, resulting in an animation depicting the sequence of events that occurred with the passage of time. Briefly, the discs with C. albicans (adhered for 90 min as above) were placed in a 35-mm-diameter glass-bottom Petri dish (MatTek Corp., Ashland, Mass.). Next, CD101 (dissolved in the growth medium) was added to the Petri dish, and incubated at 37° C. to allow formation of biofilm. Phase contrast images for this interaction were captured immediately from 0 h and followed up to 16-17 h on a Leica DMI 6000 B inverted microscope connected to a Retiga EXi Aqua camera (Q-imaging Vancouver British Columbia). To determine the structural changes in the maturing biofilm, both acquisition and analysis of a series of horizontal (xy) optical sections of the biofilm was done using Metamorph Imaging software (Molecular Devices, Downington, Pa.). Disc incubated with media alone was used as control.

Statistical Analyses

Statistical analyses for all data were performed using GraphPad Prism 6 software. Drug treated groups were compared to control untreated groups using unpaired t-tests. P-value of <0.05 was considered significant.

### Results

Activity Against Adhesion Phase Biofilms (Prevention)

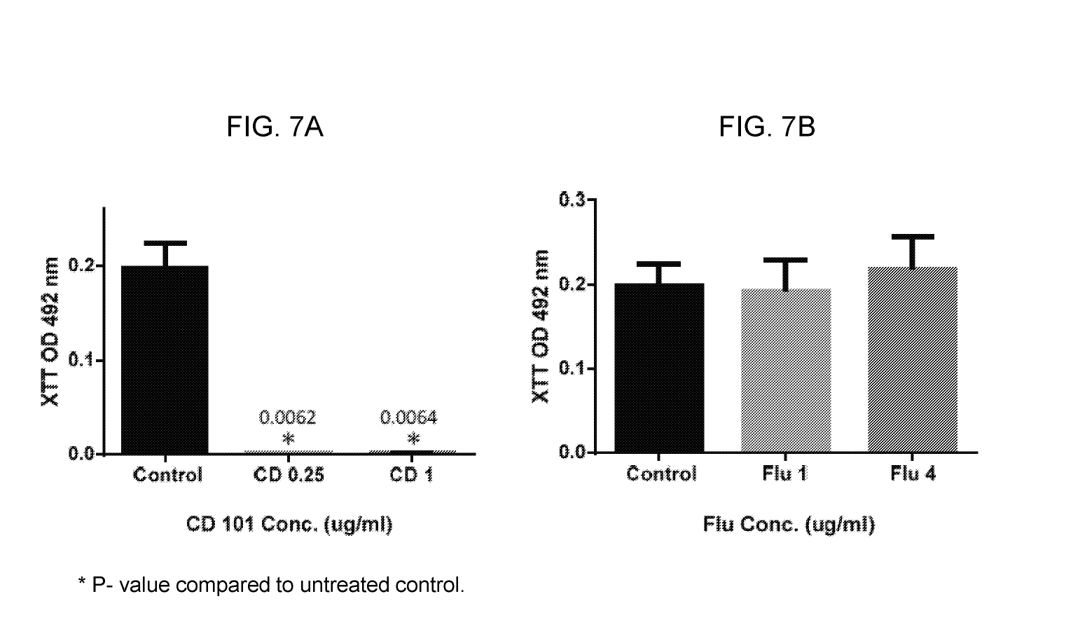

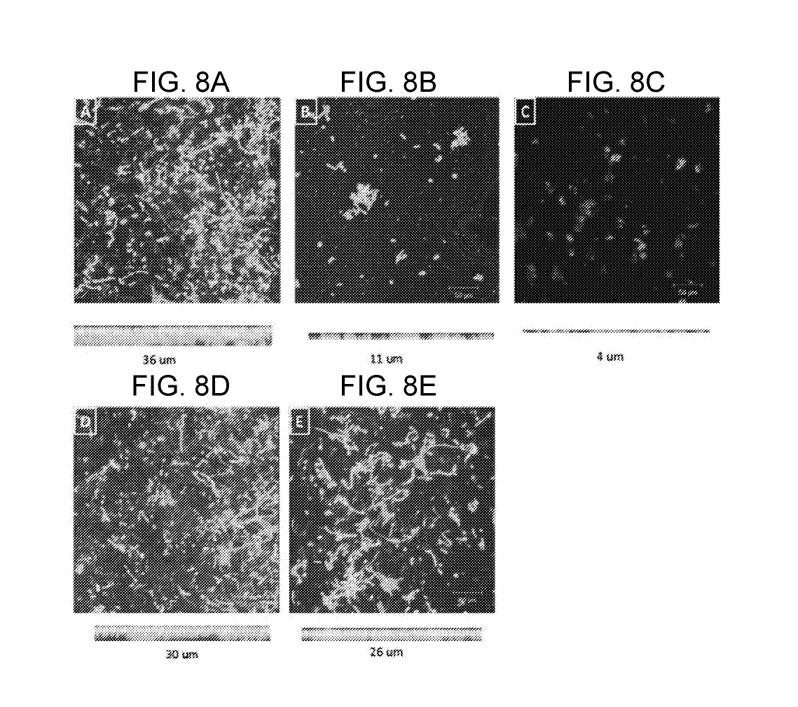

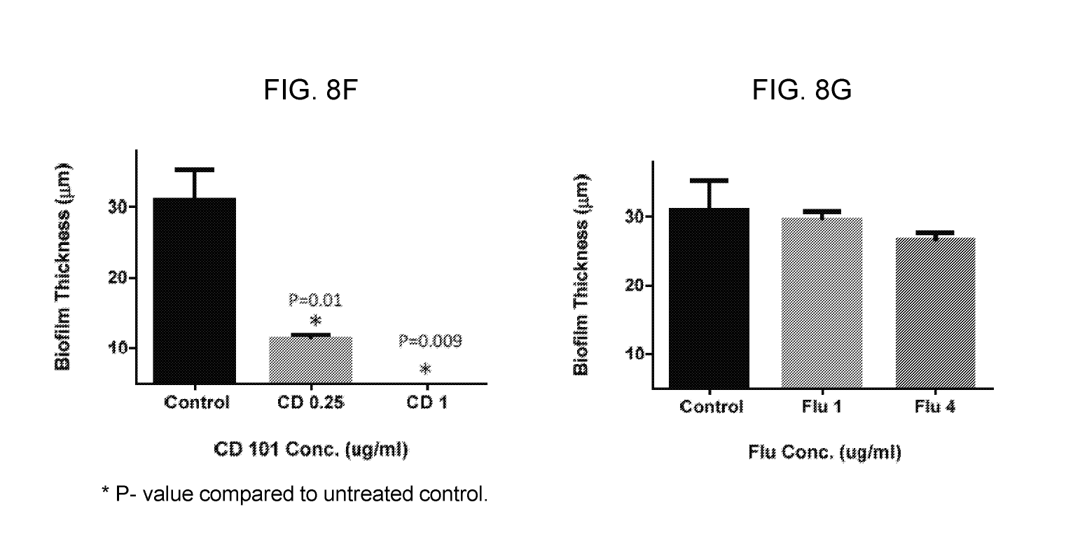

Our metabolic activity and CSLM results showed that CD101 prevented formation of robust biofilms at both concentrations tested (0.25 and 1 μg/ml). Assessment of metabolic activity revealed that C. albicans treated with CD101 formed significantly less biofilms compared to untreated C. albicans (FIG. 7A, P<0.05), In contrast, fluconazole did not inhibit biofilm formation at the two concentrations tested (1 and 4 μg/ml, FIG. 7B, P>0.05). CSLM images showed highly heterogeneous architecture of biofilms with cells/hyphae embedded within extracellular matrix for untreated control (FIG. 8A) while exposure to both concentrations of CD101 showed only remnants of adhered cells, and no biofilm maturation (FIGS. 8B and 8C). In contrast, fluconazole did not inhibit biofilm formation (FIGS. 8D and 8E). Additionally, exposure to CD101 significantly reduced the thickness of biofilms compared to untreated control (36 μm vs. 4 μm, P<0.05, FIG. 8F), while fluconazole had no effect on biofilm thickness (FIG. 8G).

Activity Against Mature Phase Biofilms (Treatment)

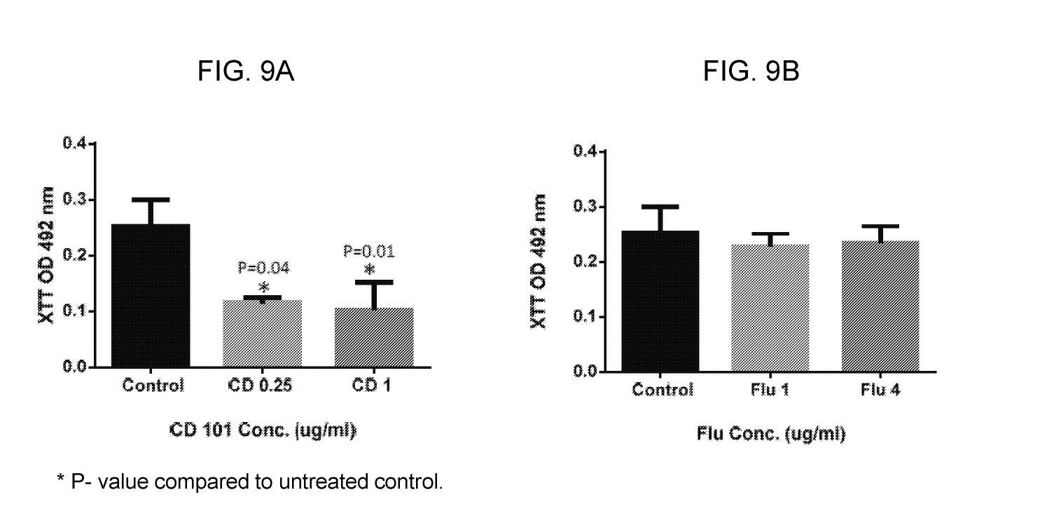

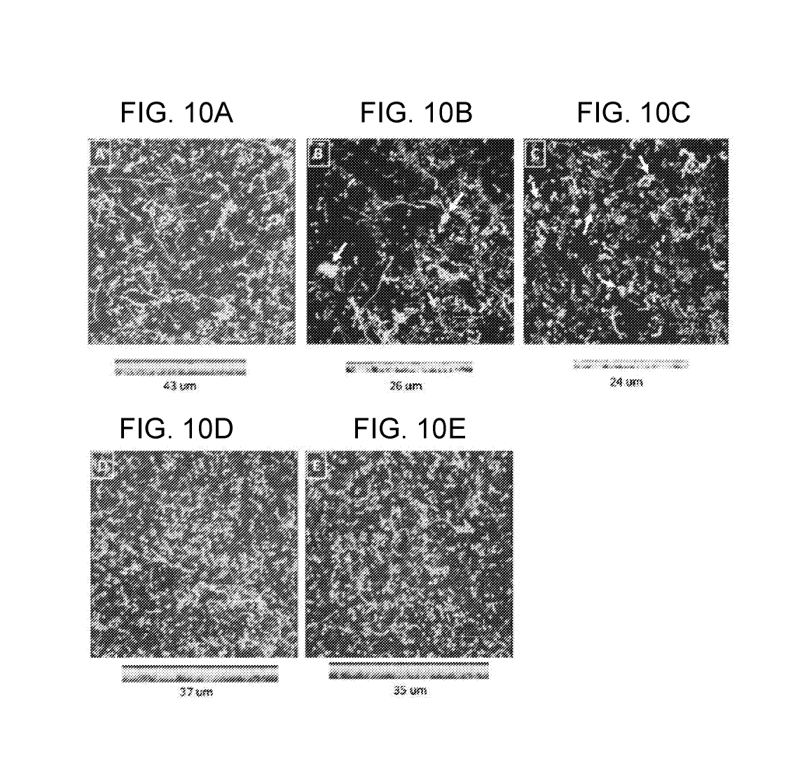

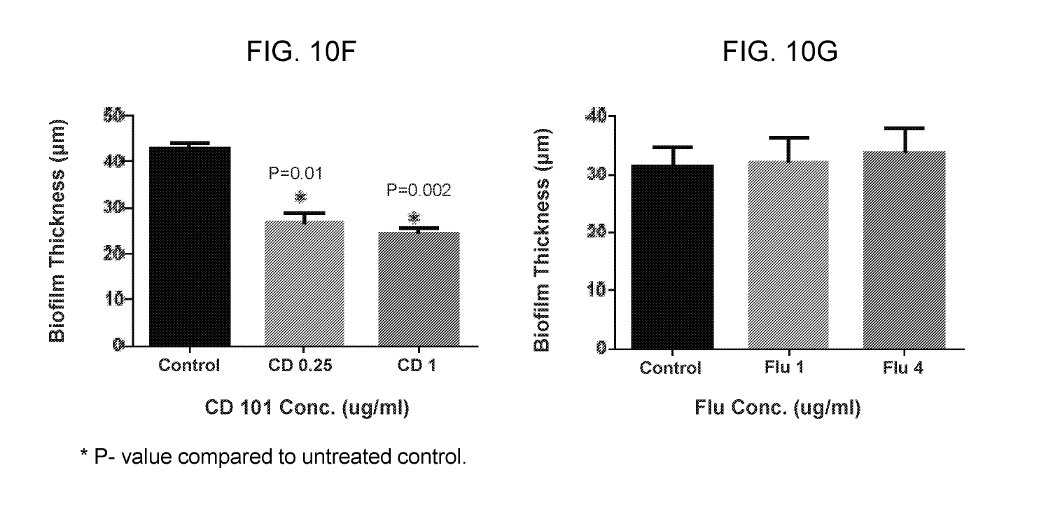

Metabolic activity and CSLM results showed that CD101 was active against mature biofilms at both tested concentrations (0.25 and 1 μg/ml). Mature C. albicans biofilms exposed to CD101 exhibited significantly less metabolic activity compared to those formed by untreated biofilms (FIG. 9A, P<0.05). In contrast, neither concentrations of fluconazole (1 and 4 μg/ml) affected these biofilms (FIG. 9B, P>0.05 compared to untreated controls). CSLM analyses showed highly heterogeneous architecture of biofilms for untreated control (FIG. 10A), while biofilm treated with CD101 were eradicated and showed bulged, deformed/broken cells (FIGS. 10B and 10C). In contrast, fluconazole did not affect Candida biofilms at both concentrations used (FIGS. 10D and 10E). Additionally, CD101 significantly reduced thickness of biofilms compared to untreated control (43 μm vs. 24 μm, P<0.05, FIG. 10F) while fluconazole had no effect (FIG. 10G).

Time Lapse

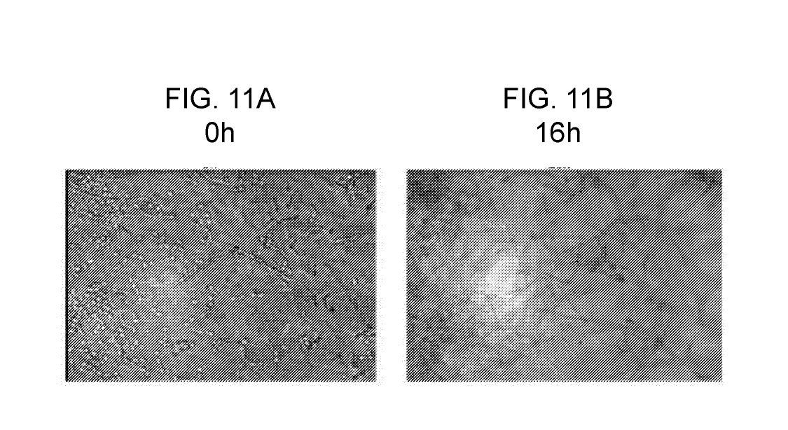

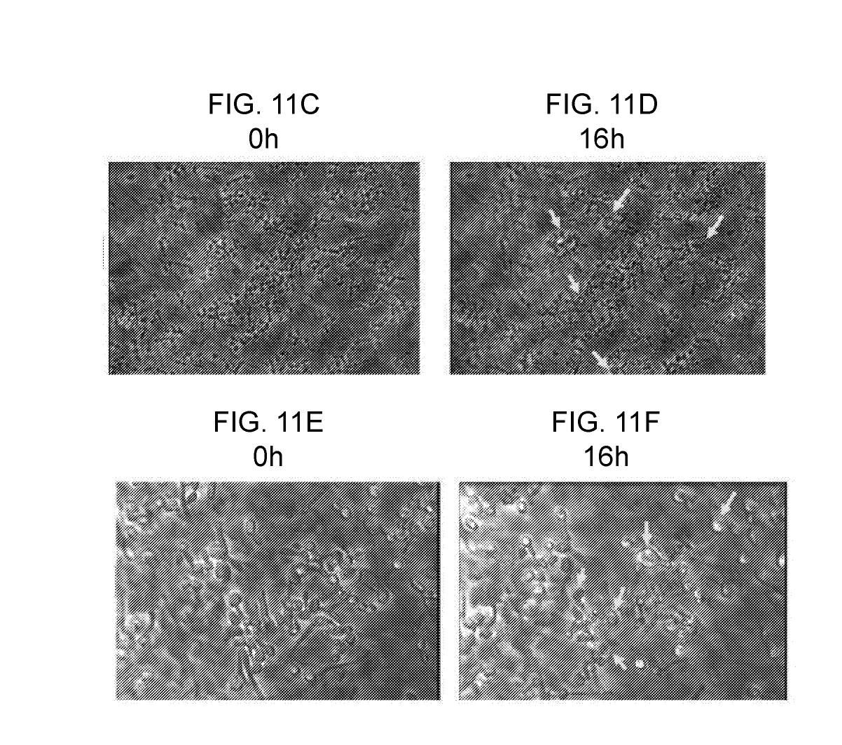

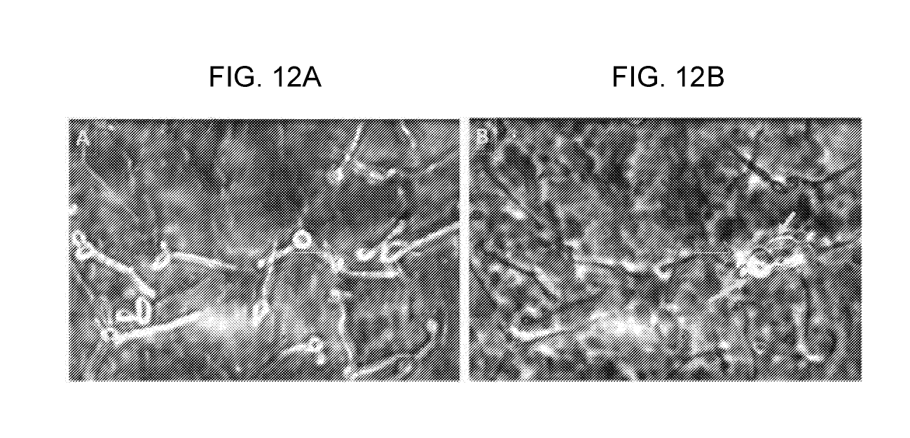

Time lapse movies showed that untreated biofilms formed highly heterogeneous architecture of biofilms with cells/hyphae embedded within extracellular matrix (screen frames in FIGS. 11A and 11B). In contrast, biofilms exposed to 0.25 μg/ml CD101 showed only adhered cells with stunted growth, which failed to grow into mature biofilms (FIGS. 11C-11F). Under high magnification, bulging, deformed, and broken cells were clearly visible (arrows, FIGS. 11D and 11F). The effect of CD101 (0.25 μg/ml) was also studied on 3 h formed biofilms and images were captured immediately after adding the drug and followed up to 16 h. Screen frames in FIG. 12A showed 3 h biofilm hyphal growth which after adding drug remained stunted and failed to grow into mature biofilms (FIG. 12B). Bulged, deformed, broken cells/hypha were clearly visible after 16 h (arrows, FIG. 12B).

### Conclusion

Our results demonstrate that CD101 possesses anti-biofilm activity against both adhesion phase and mature phase biofilms formed by C. albicans.

### Example 10: Prophylactic, Single-Dose, Subcutaneous Administration of CD101 Shows Robust Efficacy in Neutropenic Mouse Models of Candidiasis and Aspergillosis

The potential for intermittent subcutaneous (SC) administration of CD101 may extend the utility of CD101 beyond that of other echinocandins, to include antifungal treatment and prophylaxis in the outpatient setting. Neutropenic mouse models of candidiasis and aspergillosis were used to evaluate the in vivo efficacy of single SC doses of CD101 as antifungal prophylaxis.

### Method

Candidiasis model: ICR mice (5/group) were rendered neutropenic by cyclophosphamide on Day −4 (150 mg/kg) and Day −1 (100 mg/kg), then challenged (Day 0) with Candida albicans ATCC SC5314 via IV (100 μL, 10⁵ CFU/mouse). Prior to challenge, mice were given one SC dose (5, 10, or 20 mg/kg) of CD101 on Day −5, Day −3, or Day −1. At 24 hours post-challenge, kidneys were removed for CFU enumeration.

Aspergillosis model: ICR mice (6/group) were rendered neutropenic by cyclophosphamide on Day −3 (6 mg/mouse), Day +1, and Day +4 (2 mg/mouse). Challenge with Aspergillus fumigatus ATCC via IV (100 μL, 10⁴ CFU/mouse) occurred on Day 0. Prior to challenge, mice were given one SC dose (5, 10 or 20 mg/kg) of CD101 on Day −5, Day −3, or Day −1. Survival was monitored for 14 days.

### Results

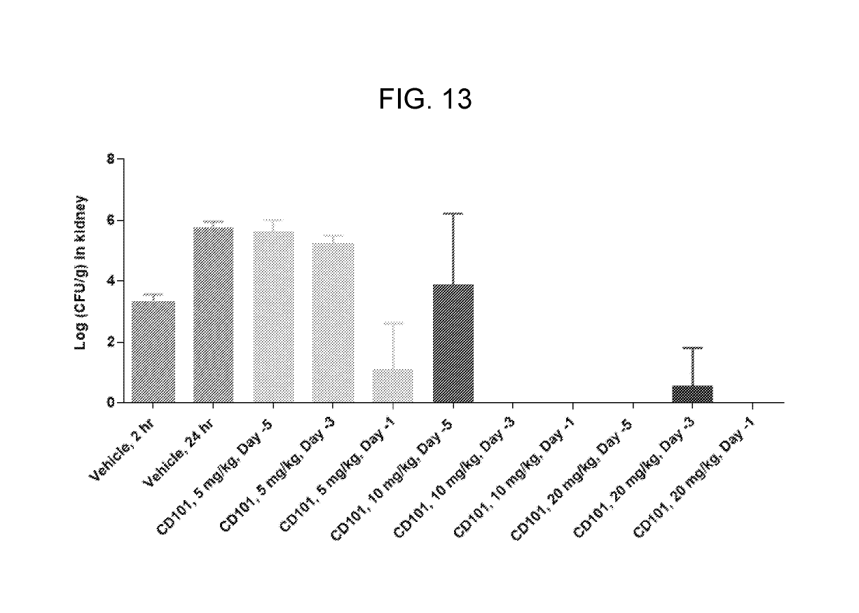

In the candidiasis model (FIG. 13), kidney CFU decreased with increasing doses of CD101 and prophylaxis occurring closer to challenge. Complete clearance was observed in all animals receiving 10 mg/kg at Day −3 and Day −1 and all but one animal receiving 20 mg/kg on Day −3. At doses of 5 or 10 mg/kg, prophylaxis with CD101 demonstrated a significant decrease in CFU at Day −3 and Day −1. At the highest dose of 20 mg/kg, CD101 reduced CFU burden regardless of prophylactic treatment day.

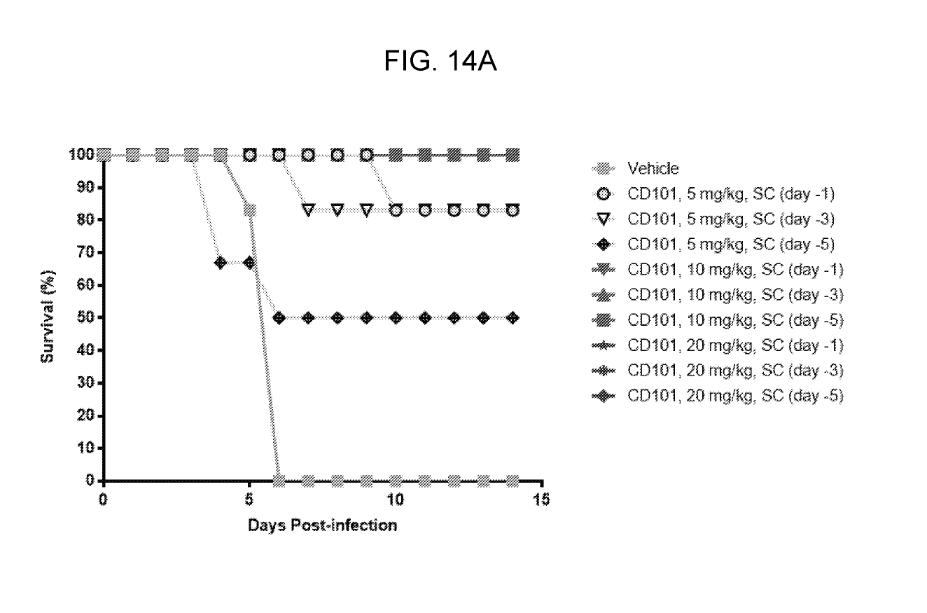

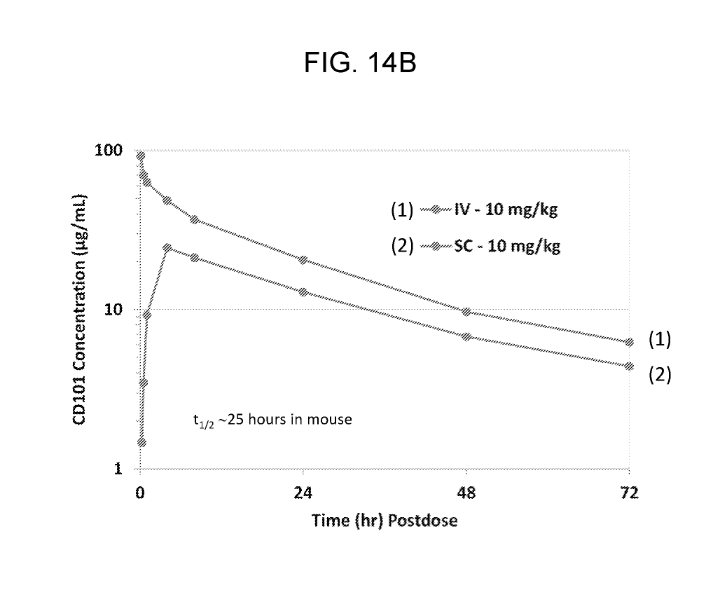

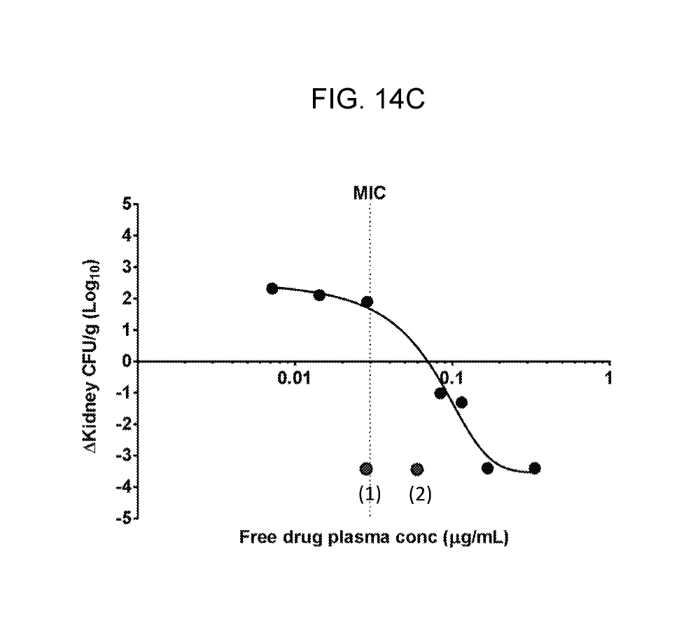

In the aspergillosis model (FIG. 14A), survival was monitored for 14 days after challenge. Subcutaneous CD101 at 5, 10, and 20 mg/kg on Day −5, Day −3 or Day −1 were associated with significant (>50%) increases in survival compared with vehicle. The 5 mg/kg group showed increased survival when prophylaxis was given closer to challenge. All animals in the 10 and 20 mg/kg groups survived regardless of prophylactic treatment day.

The pharmacokinetic profile of CD101 in mice following a 10-mg/kg subcutaneous dose shows a half-life of ˜25 hrs with an absolute bioavailability of ˜50% (FIG. 14B). The AUC from subcutaneous 10 mg/kg in mouse approximates an IV 200 mg dose in human. In general, a correlation was noted between free drug plasma concentration at time of infection over MIC (0.03 μg/mL) with higher free drug plasma concentration generating greater CFU reduction as shown in FIG. 14C for the candidiasis model. Exceptions as indicated by (1) and (2) in FIG. 14C correspond to 10 and 20 mg/kg (Day −3 and ˜5 respectively) and indicate an apparent hysteresis where effective prophylaxis occurs despite low plasma concentration likely due to slower clearance from tissues.

### Example 11: Efficacy of CD101 in the Treatment of Vulvovaginal Candidiasis in a Rat Model

### Methods and Experimental Design

Animal Strain.

Wistar rats were supplied by Harlan Laboratories UK and were specific pathogen free. Rats weighed 80-100 g at the time of surgery. Ovariectomies were performed. Rats were allowed 4-7 days recovery before transportation to the facility where the experiment was to be performed. Following arrival, rats were allowed at least 4 days acclimatization before start of the experiment. Rats weighed 100-120 g at the time of ovariectomy and were about 300 g at start of the experiment.

Animal Housing.

Rats were housed in sterilized individual ventilated cages that expose the animals at all times to HEPA filtered sterile air. Rats had free access to food and water (sterile) and had sterile aspen chip bedding (changed every 3-4 days). Additionally, during infection, rats had additional access to wet food if required to ensure they remained fully hydrated.

The room temperature was 22° C.+/−1° C., with a relative humidity of 60% and maximum background noise of 56 dB. Mice were exposed to 12 hour light/dark cycles.

Pre-Conditioning.

Female Wistar rats underwent ovariectomy at least 10 days prior to the study commencing. They were further pre-conditioned by treatment with 5 mg/kg 17-β-estradiol administered subcutaneously (SC) every other day on Days −7, −5, −3 and −1 prior to infection with C. albicans strain 529. Estradiol treatment continued every other day throughout the study to 7 days post-infection.

Yeast Isolate.

Candida albicans strain 529L was used in this chronic rat vaginal infection model.

Infection.

Yeast strains were inoculated aerobically onto Sabouraud dextrose agar media (SAB) containing 0.05 mg/mL chloramphenicol and incubated at 30° C. for 48-72 h. 18-24 h prior to infection, Yeast Peptone Dextrose (YPD) broths were inoculated with 2-3 isolated colonies from agar plates and incubated overnight (37° C. on an orbital shaker). Broths containing C. albicans strain 529L were washed 3 times with sterile phosphate buffered saline (PBS) before dilution to the correct inoculum for infection. Cell counts were determined using a haemocytometer and confirmed by quantitative culture on Sabouraud dextrose agar.

Rats were infected with 0.1 mL by intravaginal administration under inhaled isoflurane anaesthesia using about 9.8×10⁵ CFU/mL (9.8×10⁴ CFU/Rat) C. albicans strain 529L

Preparation of 17-β-estradiol, CD101, and Fluconazole

17-β-estradiol in 20% 2-hydroxypropyl-β-cyclodextrin (HPBCD). 90 mg of 17-β-estradiol (Sigma, UK) was weighed and added to 7.2 g HPBCD (Sigma, UK) and water for infection (WFI) added to obtain a final volume of 36 mL of suspension and used immediately to dose animals at 2.5 mg/mL.

Vehicle: 12.81 mg/mL Mannitol in WFI. 384.3 mg of mannitol was weighed and 30 mL of WFI was added. The mixture was briefly vortexed until completely solubilized and was filter sterilized using a 0.2 μm filter. This was stored at 2-8° C. until required and was warmed to room temperature before use.

CD101. To 61.3 mg of CD101 add 12.26 mL of vehicle and mixture briefly vortexed until completely solubilized. This was used neat at 2 mL/kg for the 10 mg/kg dose and was diluted in vehicle 1:2 to prepare the 5 mg/kg dose. These were stored at 2-8° C. until required and were allowed to warm to room temperature before use. Animals were dosed at 2 mL/kg dosing volume by the SC route.

Fluconazole. Clinical oral suspension was used to prepare fluconazole as follows: 1) Oral suspension was prepared as per manufacturer instructions (10 mg/mL Fluconazole); and 2) The 10 mg/mL oral suspension was further diluted 1:5 in WFI to give 2 mg/mL (20 mg/kg) dosing solution. This was maintained at room temperature until required and animals dosed at 10 mg/mL dosing volume orally (by the PO route).

Treatment.

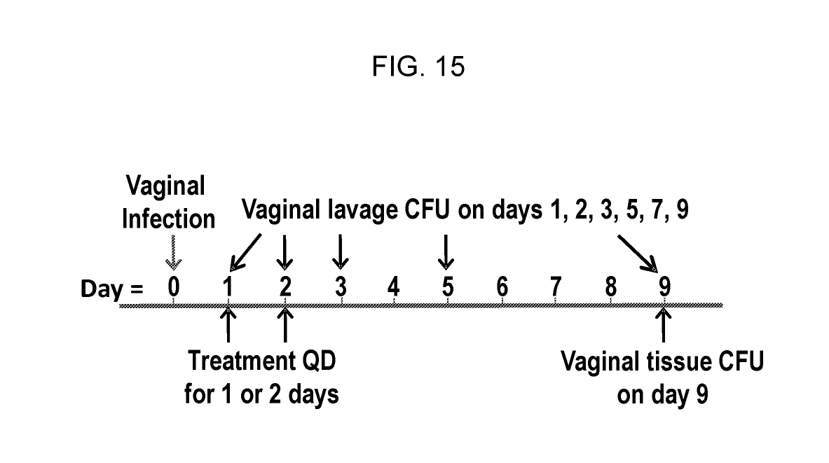

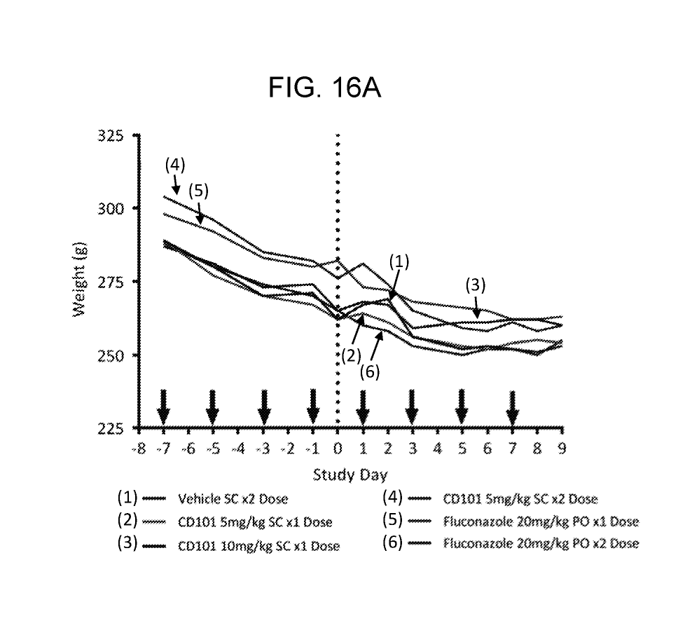

CD101, fluconazole, and vehicle treatments started at 24 h post infection by the SC route following the dose volume and frequency shown in Table 7. The fluconazole treatment also started at 24 h post infection but was administered by the PO route at the dose volume and frequency shown in Table 7. The study design is further outlined in FIG. 15.

TABLE 7

| | | | | | Number | Treatment | Total | End of | |
|---|---|---|---|---|---|---|---|---|---|
| | | | Dosing | | of | commences | Number | Experiment | |
| Group | | Dose | volume | Route/ | treatment | (hours post | of | (day post | Group |
| No. | Treatment | (mg/kg) | (mL/kg) | Regime | days | infection) | Doses | infection) | Size |
| 1 | Vehicle | — | 2 | SC OD | 2 | 24 | 2 | 9 | 6 |
| 2 | CD101 | 5 | 2 | SC OD | 1 | 24 | 1 | 9 | 6 |
| 3 | CD101 | 10 | 2 | SC OD | 1 | 24 | 1 | 9 | 6 |
| 4 | CD101 | 5 | 2 | SC OD | 2 | 24 | 2 | 9 | 6 |
| 5 | Fluconazole | 20 | 10 | PO OD | 1 | 24 | 1 | 9 | 6 |
| 6 | Fluconazole | 20 | 10 | PO OD | 2 | 24 | 2 | 9 | 6 |

Endpoints.

The rats were monitored at a frequency appropriate for their clinical condition. Rat weights were recorded at least once daily to ensure animals remained within ethical limits.

Rats do not typically succumb to infection in this model but untreated rats may experience some weight loss, dehydration, and piloerection. Reduction in weight and general loss of condition due to estradiol treatment are also typical in this rat model. Colonization with C. albicans was determined by quantitative culture of daily vaginal lavage samples. Rats were euthanized 9 days post infection and C. albicans determined by quantitative culture of vaginal tissue (including uterine horns).

Lavage samples were obtained on Days 1 (pre-treatment), 2, 3, 5, 7, and 9 days post infection by flushing rat vaginas 4 times with 0.1 mL pre-warmed sterile PBS. Following euthanasia, vaginal tissue including uterine horns was removed prior to weighing. Tissues were homogenized in 2 mL sterile PBS using a bead-beater. Vaginal wash and tissue homogenates were diluted appropriately then quantitatively cultured on to Sabouraud dextrose agar containing 0.05 mg/mL chloramphenicol and incubated at 37° C. for up to 72 h before being counted.

Statistical Analysis.

Data were analysed using StatsDirect software (version 2.7.8) using the non-parametric Kruskal-Wallis test and if this was statistically significant all pairwise comparisons were analysed (Conover-Inman).

### Results

In this study, the in vivo efficacy of CD101 dosed SC once at 5 and 10 mg/kg or dosed SC twice at 5 mg/kg was investigated in a rat model of vulvovaginal candidiasis caused by C. albicans strain 529L.

CD101 and Fluconazole Tolerability and Clinical Condition.

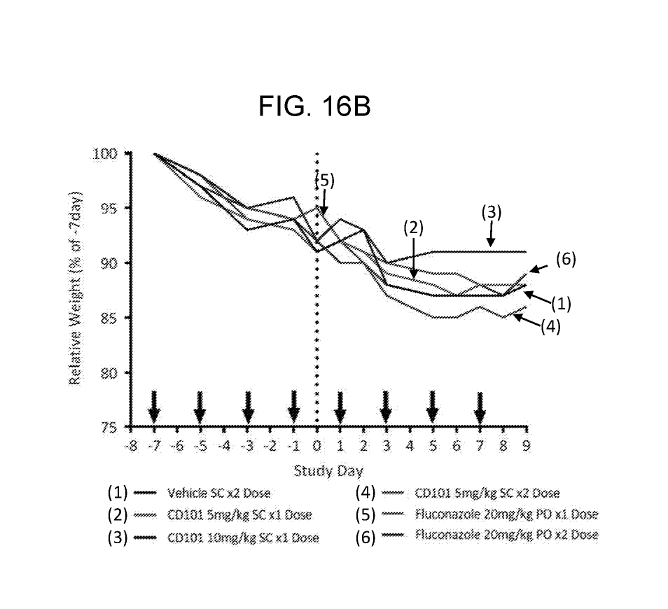

CD101 and fluconazole at all treatment dose and duration were well tolerated with no adverse events observed. Animal weights following localized vaginal infection with C. albicans and treatment with CD101 or fluconazole are shown in FIGS. 16A and 16B. Animal weights are shown as daily group average weights (FIG. 16A) and the weight relative to that measured on day of infection (Day 0, FIG. 16B). As is typical of this model, ovariectomised rats slowly lost weight following multiple doses of 1713-estradiol. Weight loss observed was typical of the model and did not seem to be exacerbated by CD101 treatment.

Pharmacokinetic Profile of CD101.

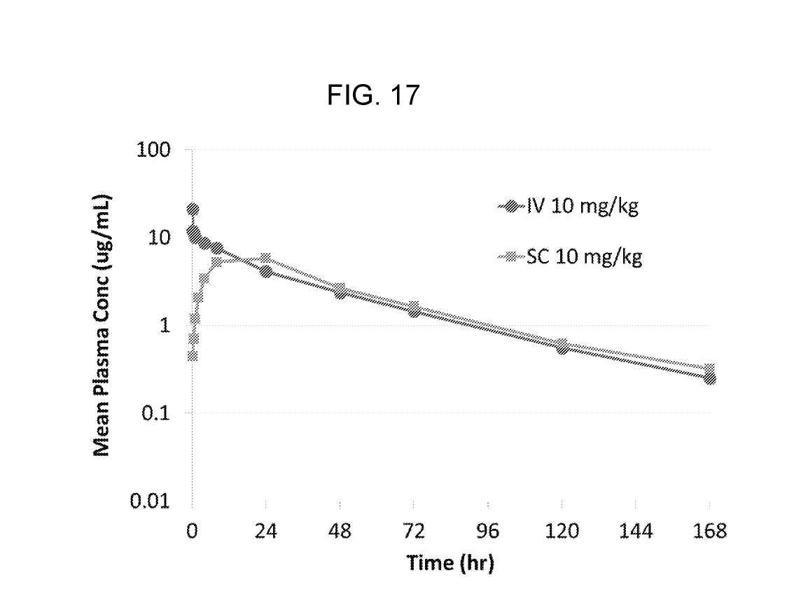

The pharmacokinetic (PK) profile of CD101 in female rats (three per group) was characterized. Following subcutaneous (SC) administration, the time to Cmax (i.e. Tmax) was observed between 8 to 24 hours post-dose suggesting slow absorption/distribution from the site of administration (FIG. 17). The half-life, t½, values were similar to those observed from intravenous (IV) dosing and shows a t½ of 48 hrs and SC bioavailability of 97%.

Efficacy Data.

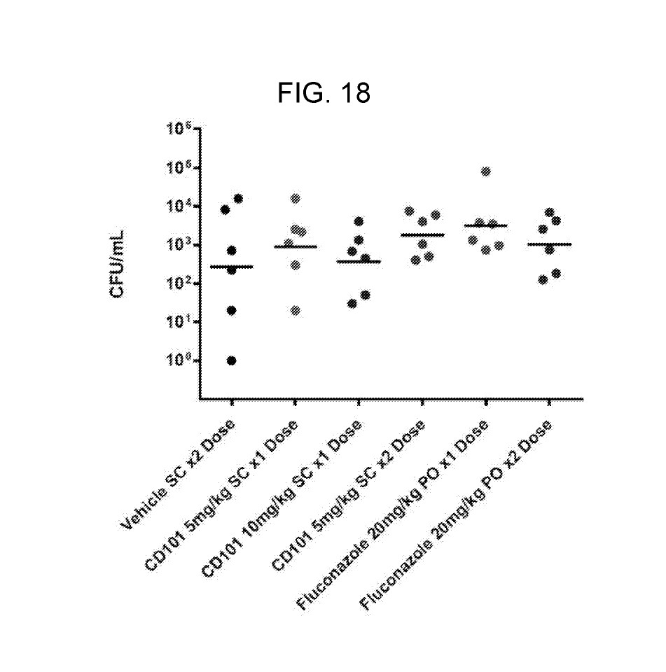

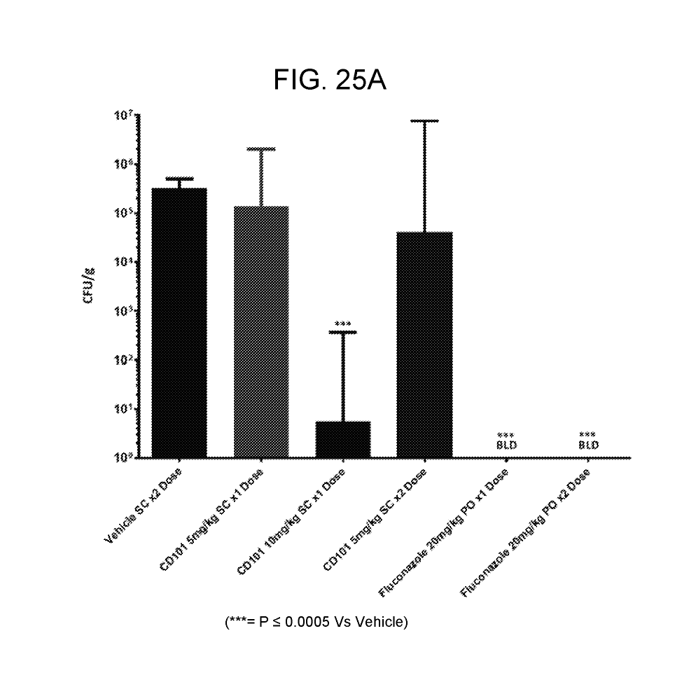

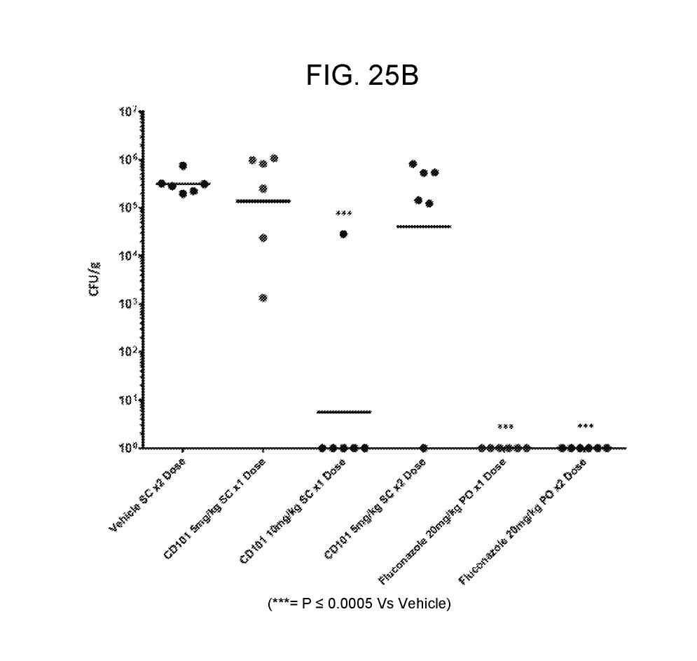

A robust model of localized C. albicans strain 529L vaginal infection was successfully established. The geometric mean burden of all infected rats was about 0.9×10³ CFU/mL on Day 1 post infection in pre-treatment vaginal lavage samples (Table 8 and FIG. 18). This level of infection recovered from vehicle only lavage samples increased slightly (about 2.0 Log₁₀ CFU/mL) over the duration of the study and was stable between Day 5 to end of study. A high level (about 5.5 Log₁₀ CFU/g) of C. albicans burden was also obtained from terminal vagina, uterus, and uterine horn tissue taken from vehicle control rats at the end of the study (see Table 14 and FIGS. 25A and 25B) as is typical of this model (the fungi are tightly adherent to the vaginal mucosa due to pseudo-hyphal invasion).

TABLE 8
Geometric mean burden on Day 1 post infection
in pre-treatment vaginal lavage samples

| | Group | | Log Group | Log reduction |
|---|---|---|---|---|
| | Geometric | Standard | Geometric | from vehicle |
| | mean | Deviation | mean | control |
| Treatment | (CFU/mL) | (CFU/mL) | (CFU/mL) | (CFU/mL) |
| Vehicle SC - | 2.74 × 10² | 6.60 × 10³ | 2.44 | 0.00 |
| Dosed Twice | | | | |
| CD101 5 mg/ | 9.11 × 10² | 6.11 × 10³ | 2.96 | −0.52 |
| kg SC - | | | | |
| Dosed Once | | | | |
| CD101 10 mg/ | 3.68 × 10² | 1.53 × 10³ | 2.57 | −0.13 |
| kg SC - | | | | |
| Dosed Once | | | | |
| CD101 5 mg/ | 1.83 × 10³ | 3.06 × 10³ | 3.26 | −0.83 |
| kg SC - | | | | |
| Dosed Twice | | | | |
| Fluconazole | 3.16 × 10³ | 3.18 × 10⁴ | 3.50 | −1.06 |
| 20 mg/kg PO - | | | | |
| Dosed Once | | | | |
| Fluconazole | 1.04 × 10³ | 2.73 × 10³ | 3.02 | −0.58 |
| 20 mg/kg PO - | | | | |
| Dosed Twice | | | | |

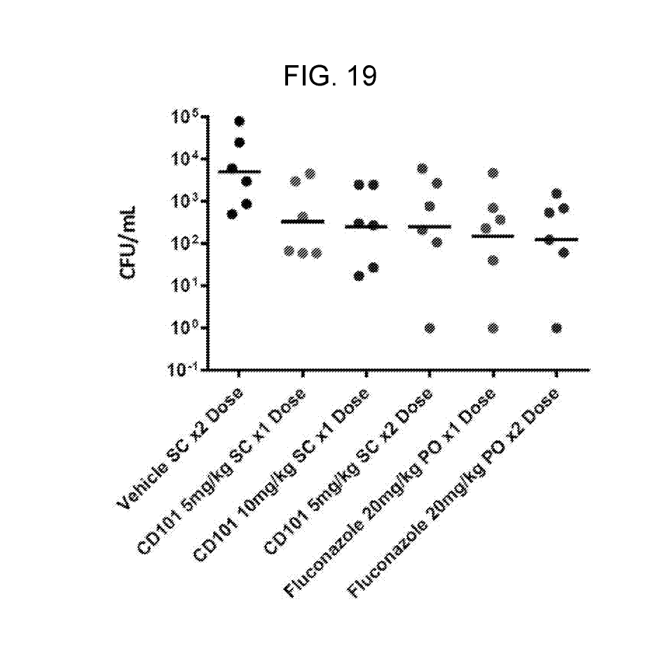

The daily lavage data showed the following results:
Day 1 post treatment (Day 2 post infection, Table 9 and FIG. 19)—All doses of CD101 showed similar reduction in burden (about 1.3 Log₁₀ CFU/mL). Fluconazole showed slightly higher reduction (about 1.6 Log₁₀ CFU/mL). However, neither the CD101 nor fluconazole burden was statistically different from the vehicle burden.

TABLE 9
Geometric mean burden on Day 1 post
treatment (Day 2 post infection)

| | Group | | Log Group | Log reduction |
|---|---|---|---|---|
| | Geometric | Standard | Geometric | from vehicle |
| | mean | Deviation | mean | control |
| Treatment | (CFU/mL) | (CFU/mL) | (CFU/mL) | (CFU/mL) |
| Vehicle SC - | 5.00 × 10³ | 3.12 × 10⁴ | 3.70 | 0.00 |
| Dosed Twice | | | | |
| CD101 5 mg/ | 3.34 × 10² | 1.92 × 10³ | 2.52 | 1.17 |
| kg SC - | | | | |
| Dosed Once | | | | |
| CD101 10 mg/ | 2.48 × 10² | 1.22 × 10³ | 2.39 | 1.30 |
| kg SC - | | | | |
| Dosed Once | | | | |
| CD101 5 mg/ | 2.56 × 10² | 2.37 × 10³ | 2.41 | 1.29 |
| kg SC - | | | | |
| Dosed Twice | | | | |
| Fluconazole | 1.50 × 10² | 1.83 × 10³ | 2.18 | 1.52 |
| 20 mg/kg PO - | | | | |
| Dosed Once | | | | |
| Fluconazole | 1.27 × 10² | 5.83 × 10² | 2.10 | 1.59 |
| 20 mg/kg PO - | | | | |
| Dosed Twice | | | | |

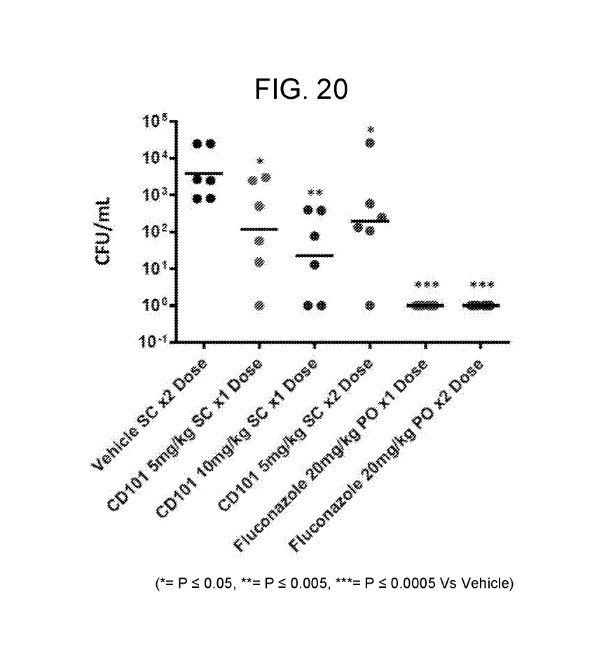

Day 2 post treatment (Day 3 post infection, Table 10 and FIG. 20)—CD101 dosed once at 10 mg/kg showed a higher reduction in burden compared to CD101 dosed once or twice at 5 mg/kg. The CD101 burden data was more variable compared to vehicle or fluconazole treated animals. Fluconazole showed the greatest reduction in burden with 11/12 rats having burden below the limit of detection. All CD101 and fluconazole treatments were statistically different from the vehicle treatments.

TABLE 10
Geometric mean burden on Day 2 post
treatment (Day 3 post infection)

| | Group | | Log Group | Log reduction |
|---|---|---|---|---|
| | Geometric | Standard | Geometric | from vehicle |
| | mean | Deviation | mean | control |
| Treatment | (CFU/mL) | (CFU/mL) | (CFU/mL) | (CFU/mL) |
| Vehicle SC - | 3.74 × 10³ | 1.21 × 10⁴ | 3.57 | 0.00 |
| Dosed Twice | | | | |
| CD101 5 mg/ | 1.21 × 10² | 1.37 × 10³ | 2.08 | 1.49 |
| kg SC - | | | | |
| Dosed Once | | | | |
| CD101 10 mg/ | 23.2 | 1.92 × 10² | 1.37 | 2.21 |
| kg SC - | | | | |
| Dosed Once | | | | |
| CD101 5 mg/ | 1.95 × 10² | 1.07 × 10⁴ | 2.29 | 1.28 |
| kg SC - | | | | |
| Dosed Twice | | | | |
| Fluconazole | 1.00 | 0.00 | 0.00 | 3.57 |
| 20 mg/kg PO - | | | | |
| Dosed Once | | | | |
| Fluconazole | 1.49 | 4.08 | 0.17 | 3.40 |
| 20 mg/kg PO - | | | | |
| Dosed Twice | | | | |

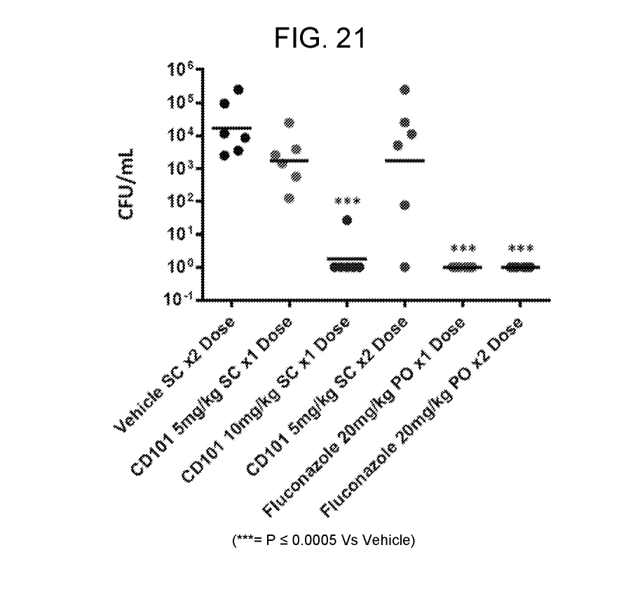

Day 4 post treatment (Day 5 post infection, Table 11 and FIG. 21)—CD101 dosed at 5 mg/kg once or twice reduced the burden to about 1 Log₁₀ CFU/m but this was not statistically different from the vehicle treatments. CD101 dosed once at 10 mg/kg was highly efficacious and reduced the burden to about 4 Log₁₀ CFU/mL with 5/6 rats having burden below the limit of detection and almost similar to the fluconazole. All rats treated with the fluconazole once or twice had burdens that were below the limit of detection.

TABLE 11
Geometric mean burden on Day 4 post
treatment (Day 5 post infection)

| | Group | | Log Group | Log reduction |
|---|---|---|---|---|
| | Geometric | Standard | Geometric | from vehicle |
| | mean | Deviation | mean | control |
| Treatment | (CFU/mL) | (CFU/mL) | (CFU/mL) | (CFU/mL) |
| Vehicle SC - | 1.65 × 10⁴ | 9.88 × 10⁴ | 4.22 | 0.00 |
| Dosed Twice | | | | |
| CD101 5 mg/ | 1.70 × 10³ | 9.41 × 10³ | 3.23 | 0.99 |
| kg SC - | | | | |
| Dosed Once | | | | |
| CD101 10 mg/ | 1.73 | 10.6 | 0.24 | 3.98 |
| kg SC - | | | | |
| Dosed Once | | | | |
| CD101 5 mg/ | 1.73 × 10³ | 9.91 × 10⁴ | 3.24 | 0.98 |
| kg SC - | | | | |
| Dosed Twice | | | | |
| Fluconazole | 1.00 | 0.00 | 0.00 | 4.22 |
| 20 mg/kg PO - | | | | |
| Dosed Once | | | | |
| Fluconazole | 1.00 | 0.00 | 0.00 | 4.22 |
| 20 mg/kg PO - | | | | |
| Dosed Twice | | | | |

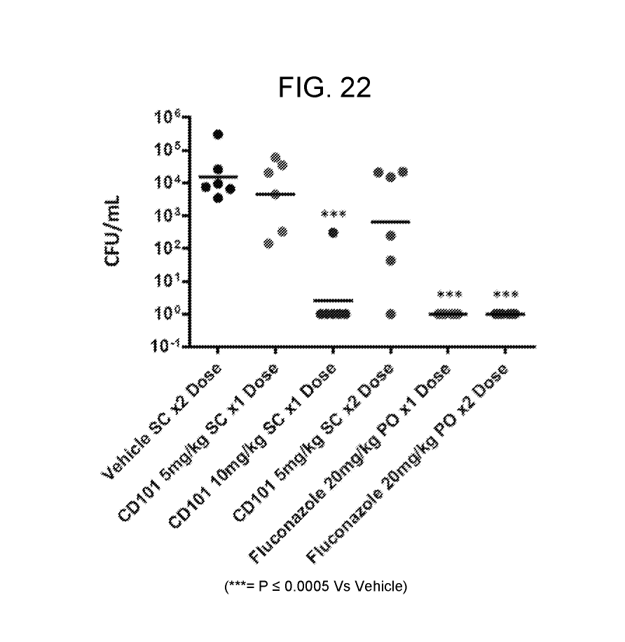

Day 6 post treatment (Day 7 post infection, Table 12 and FIG. 22)—CD101 dosed once at 5 mg/kg showed slight increase in fungal burden compared to Day 5 post infection. CD101 dosed twice at 5 mg/kg reduced the burden more than Day 5 post infection but was not statistically significant. CD101 dosed at 10 mg/kg was similar to Day 5 post infection but the overall burden reduction was not as high as Day 5. The single rat with detectable burden on Day 5 had a slightly increased fungal burden.

TABLE 12
Geometric mean burden on Day 6 post
treatment (Day 7 post infection)

| | Group | | Log Group | Log reduction |
|---|---|---|---|---|
| | Geometric | Standard | Geometric | from vehicle |
| | mean | Deviation | mean | control |
| Treatment | (CFU/mL) | (CFU/mL) | (CFU/mL) | (CFU/mL) |
| Vehicle SC - | 1.53 × 10⁴ | 1.18 × 10⁵ | 4.18 | 0.00 |
| Dosed Twice | | | | |
| CD101 5 mg/ | 4.56 × 10³ | 2.39 × 10⁴ | 3.66 | 0.52 |
| kg SC - | | | | |
| Dosed Once | | | | |
| CD101 10 mg/ | 2.59 | 1.22 × 10² | 0.41 | 3.77 |
| kg SC - | | | | |
| Dosed Once | | | | |
| CD101 5 mg/ | 6.47 × 10² | 1.08 × 10⁴ | 2.81 | 1.37 |
| kg SC - | | | | |
| Dosed Twice | | | | |
| Fluconazole | 1.00 | 0.00 | 0.00 | 4.18 |
| 20 mg/kg PO - | | | | |
| Dosed Once | | | | |
| Fluconazole | 1.00 | 0.00 | 0.00 | 4.18 |
| 20 mg/kg PO - | | | | |
| Dosed Twice | | | | |

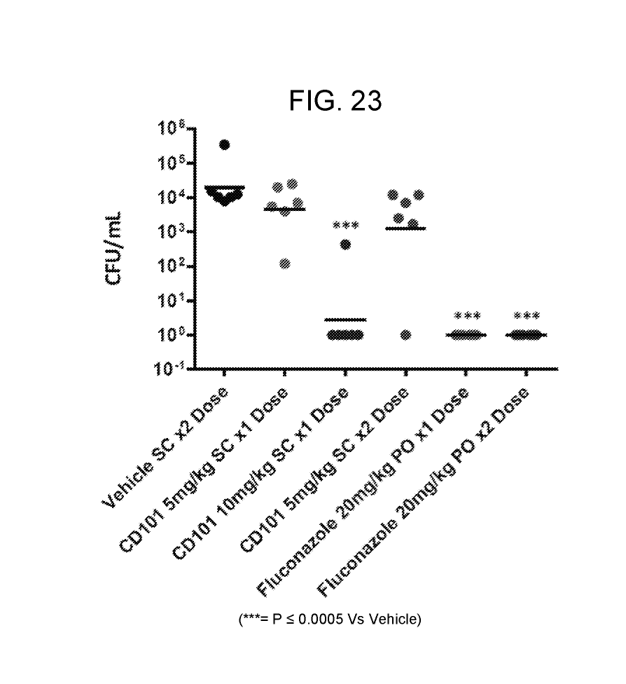

Day 8 post treatment (Day 9 post infection, Table 13 and FIG. 23)—Data were similar to Day 6 post treatment.

TABLE 13
Geometric mean burden on Day 8 post
treatment (Day 9 post infection)

| | Group | | Log Group | Log reduction |
|---|---|---|---|---|
| | Geometric | Standard | Geometric | from vehicle |
| | mean | Deviation | mean | control |
| Treatment | (CFU/mL) | (CFU/mL) | (CFU/mL) | (CFU/mL) |
| Vehicle SC - | 1.98 × 10⁴ | 1.38 × 10⁵ | 4.30 | 0.00 |
| Dosed Twice | | | | |
| CD101 5 mg/ | 4.58 × 10³ | 9.87 × 10³ | 3.66 | 0.64 |
| kg SC - | | | | |
| Dosed Once | | | | |
| CD101 10 mg/ | 2.75 | 1.76 × 10² | 0.44 | 3.86 |
| kg SC - | | | | |
| Dosed Once | | | | |
| CD101 5 mg/ | 1.28 × 10³ | 5.28 × 10³ | 3.11 | 1.19 |
| kg SC - | | | | |
| Dosed Twice | | | | |
| Fluconazole | 1.00 | 0.00 | 0.00 | 4.30 |
| 20 mg/kg PO - | | | | |
| Dosed Once | | | | |
| Fluconazole | 1.00 | 0.00 | 0.00 | 4.30 |
| 20 mg/kg PO - | | | | |
| Dosed Twice | | | | |

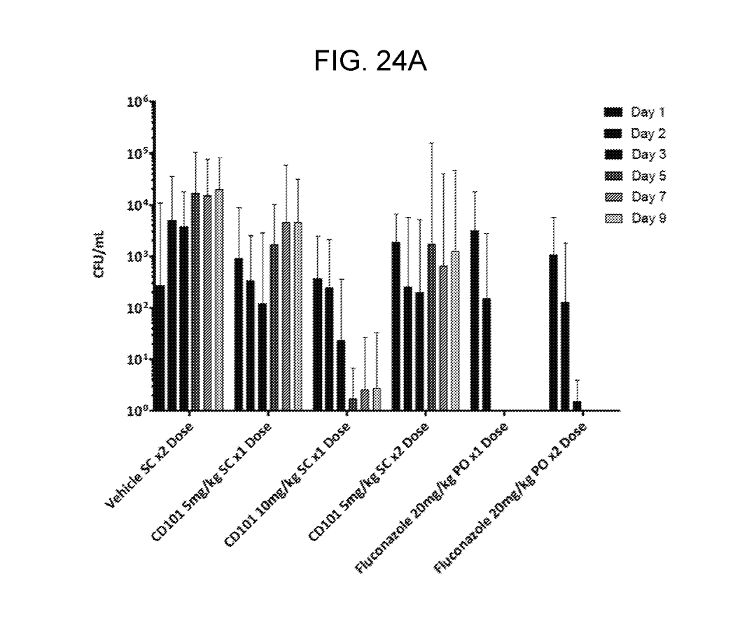

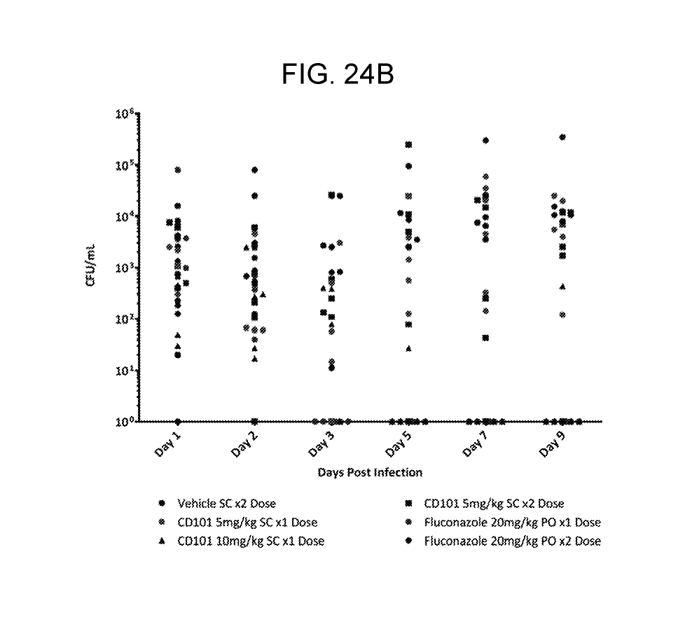

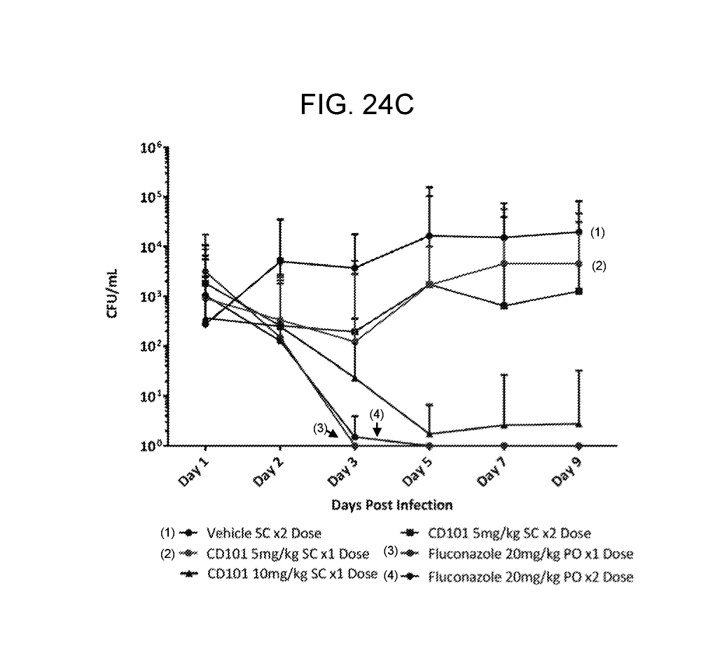

The lavage burden data are summarized in FIGS. 24A-24C. A robust VVC model was established (FIG. 24C); vehicle-treated rats maintained a high fungal burden throughout the study, rising to 2×10⁴ CFU/mL by Day 9 post infection. CD101 administered once at 10 mg/kg was the most effective dose and similar to fluconazole dosed at 20 mg/kg showing comparable CFU by Day 5 post infection and thereafter the burden increased slightly. The increase was caused by a single rat that had a small fungal burden on Day 5 but which increased on Day 7 and 9 post infection. As expected, Day 9 tissue CFU were higher than lavage CFU for all treatments, but the overall pattern was similar to lavage CFU. All but one rat treated with CD101 once at 10 mg/kg had undetectable CFU.

The terminal vaginal tissue burdens (vagina, uterus, and uterine horns) are shown in Table 14 and FIGS. 25A and 25B. The data is in line with that observed in the vaginal lavage washes. The data showed that CD101 dosed once at 5 mg/kg resulted in the smallest reduction in burden (about 0.4 Log₁₀ CFU/g) followed by CD101 dosed twice at 5 mg/kg (about 0.9 Log₁₀ CFU/g), neither were statistically lower than vehicle treatments. 5/6 rats treated with CD101 dosed once at 10 mg/kg had burdens below the levels of detection. A single rat had low level of burden. All rats treated with fluconazole once or twice had burdens below the limit of detection.

TABLE 14
Terminal vaginal tissue burden (vagina, uterus,
and uterine horns) (Day +9 post infection)

| | Group | | Log Group | Log reduction |
|---|---|---|---|---|
| | Geometric | Standard | Geometric | from vehicle |
| | mean | Deviation | mean | control |
| Treatment | (CFU/g) | (CFU/g) | (CFU/g) | (CFU/g) |
| Vehicle SC - | 3.11 × 10⁵ | 2.05 × 10⁵ | 5.49 | 0.00 |
| Dosed Twice | | | | |
| CD101 5 mg/ | 1.37 × 10⁵ | 4.88 × 10⁵ | 5.14 | 0.36 |
| kg SC - | | | | |
| Dosed Once | | | | |
| CD101 10 mg/ | 5.52 | 1.16 × 10⁴ | 0.74 | 4.75 |
| kg SC - | | | | |
| Dosed Once | | | | |
| CD101 5 mg/ | 4.00 × 10⁴ | 3.16 × 10⁵ | 4.60 | 0.89 |
| kg SC - | | | | |
| Dosed Twice | | | | |
| Fluconazole | 1.00 | 0.00 | 0.00 | 5.49 |
| 20 mg/kg PO - | | | | |
| Dosed Once | | | | |
| Fluconazole | 1.00 | 0.00 | 0.00 | 5.49 |
| 20 mg/kg PO - | | | | |
| Dosed Twice | | | | |

### Summary

A robust model of localized rat chronic model of vulvovaginal candidiasis following infection with C. albicans strain 529L was established. The infectious burden peaked to >4 Log₁₀ CFU/mL in lavage samples from the vehicle treatment group. A high level of C. albicans burden was also recovered from vagina, uterus, and uterine horn tissue taken from vehicle control rats.

Treatment with CD101 administered by the SC route showed the following in lavage wash burdens:
A single dose of 5 mg/kg administered 24 h post infection reduced fungal burden with a peak effect at Day 3 post infection (48 h post treatment) but which was not maintained to the end of the study. Burden reduction was statistically significant vs. vehicle controls only at Day 3 post infection.
A single dose of 5 mg/kg given twice; once at 24 h and another at 48 h post infection resulted in a superior burden reduction compared to the single dose which was maintained for the duration of the study. But like the single dose, statistical significance was only observed at Day 3 post infection.
A single dose at 10 mg/kg (equivalent to 200 mg in human) given at 24 h post infection resulted in substantial reduction in fungal burden at 3 day post infection (48 h post treatment) with a peak effect at 5 day post infection. Thereafter burden appeared to increase again but this was due to a single rat that retained burden whereas all others had actually burdens below the level of detection. These results suggest excellent CD101 distribution/penetration into vaginal mucosa via a single SC dose.

Treatment with CD101 administered by the SC route showed the following vaginal tissue burden:
A single dose at 5 mg/kg 24 h post infection resulted in a slight reduction in burden.
A single dose at 5 mg/kg given twice; once at 24 h and another at 48 h post infection (total of 2 doses) resulted in a larger decrease in burden compared to the single dose.
A single dose at 10 mg/kg (equivalent to 200 mg in human) given at 24 h post infection resulted in substantial reduction in burden with clearance in fungal burden to below the level of detection in 5 of the 6 rats. Similar to the lavage data, a single rat retained fungal burden. These results suggest excellent CD101 distribution/penetration into vaginal mucosa via a single SC dose.

All rats treated with fluconazole at 20 mg/kg PO dosed once at 24 h post infection or single dose twice (24 h and 48 h post infection) showed a faster reduction in burden from lavage washes compared to CD101. At the end of the study, all rats had cleared the infection to below the level of detection in both the lavage wash and vaginal tissue.

### Example 12: Pharmacodynamics of the Long Acting Echinocandin, CD101, in the Neutropenic Invasive Candidiasis Murine Model Using an Extended Interval Dosing Design

The current studies included pharmacokinetic/pharmacodynamic (PK/PD) evaluation of CD101 efficacy in a neutropenic murine model of disseminated candidiasis to assist with further clinical development of optimal dosing strategies. The studies were specifically designed to examine [1] pharmacokinetics of extended interval dosing in the murine model; [2] the CD101 dose-response relationships against a diverse group of strains including C. albicans, C. glabrata, and C. parapsilosis; [3] the PK/PD target exposures associated with efficacy against each species. Pharmacokinetic (PK)/pharmacodynamic (PD) targets were examined to provide a framework for further development of clinical dosing regimens, optimize therapy, and assist in establishment of preliminary susceptibility breakpoints.

### Methods

Antifungal Agent.

CD101 dose solutions were prepared on the day of experimentation according to manufacturer instructions with 0.9% NaCl, 10% DMSO, and 1% Tween-20.

Strains.

Ten clinical Candida strains were used for the in vivo treatment studies, including four C. albicans, three C. glabrata, and three C. parapsilosis strains (Table 15). This group was selected to encompass phenotypic variability in susceptibility to triazoles and echinocandins and based on similar fitness in the animal model as defined by the amount of growth in control animals over 24 h. The organisms were maintained, grown, and quantified on Sabouraud's dextrose agar (SDA) plates. Select strains used in the study are summarized in Table 15.

TABLE 15
Study organisms, CD101 susceptibility results, and comparative
susceptibility results to anidulafungin.

| | | CD101 MIC | Anidulafungin MIC |
|---|---|---|---|
| Organism | Strain | (mg/L) | (mg/L) |
| C. albicans | K-1 | 0.06 | 0.015 |
| | 580 | 0.06 | 0.015 |
| | 98-17 | 0.06 | 0.03 |
| | 98-210 | 0.03 | 0.015 |
| C. glabrata | 10956 | 1 | 1 |
| | 5592 | 0.125 | 0.06 |
| | 35315 | 0.5 | 0.25 |
| C. parapsilosis | 20519.069 | 1 | 4 |
| | 20477.048 | 1 | 2 |
| | 20423.072 | 0.5 | 1 |

In vitro susceptibility testing. All isolates were tested in accordance with the standards in CLSI document M27-A3. The MICs were determined visually after 24 h of incubation as the lowest concentration of drug that causes a significant diminution (50%) of growth compared to controls. MICs were determined on three separate occasions in duplicate. Results are expressed as the median of these results.

Animals.

Six-week-old ICR Swiss/CD1 specific-pathogen-free female mice (Harlan Sprague-Dawley, Indianapolis, Ind.) weighing 23 to 27 g were used for all the studies.

Infection Model.

A neutropenic, murine, disseminated candidiasis model was used for the treatment studies. The mice were rendered neutropenic (polymorphonuclear cell count, <100/mm3) by injecting 150 mg/kg of cyclophosphamide (Mead Johnson Pharmaceuticals, Evansville, Ind.) subcutaneously 4 days before infection, 100 mg/kg of cyclophosphamide 1 day before infection, and additional cyclophosphamide doses (100 mg/kg) on Day 2 and Day 4 after infection to ensure neutropenia throughout the 168 h (7 d) study period. Three mice were included in each treatment and control group.

The organisms were subcultured on SDA plates 24 h prior to infection. The inoculum was prepared by placing 3 to 5 colonies into 5 ml of sterile pyrogen-free 0.15 M NaCl warmed to 35° C. The final inoculum was adjusted to a 0.6 transmittance at 530 nm. The fungal count of the inoculum determined by viable counts on SDA was 6.1±0.2 log₁₀ CFU/ml.

Disseminated infection with the Candida strains was achieved by injection of 0.1 ml of the inoculum via the lateral tail vein 2 h prior to the start of antifungal therapy. At the end of the study period, the animals were sacrificed by CO₂ asphyxiation. The kidneys of each mouse were aseptically removed and placed in 0.15 M NaCl at 4° C. The kidneys were homogenized and serially diluted 1:10, and the aliquots were plated onto SDA for viable fungal colony counts after incubation for 24 h at 35° C. The lower limit of detection was 100 CFU/ml. The results are expressed as the mean CFU/kidney for three mice.

Pharmacokinetics.

Single-dose pharmacokinetic (PK) evaluation was undertaken following intraperitoneal (IP) doses of 1, 4, 16, and 64 mg/kg of CD101. Plasma from groups of three mice per time point (1, 3, 6, 12, 24, 48, and 72-h) was collected. The plasma drug concentrations were determined by liquid chromatography-tandem mass spectrometry. A noncompartmental model was used in the pharmacokinetic analysis. Elimination half-life was calculated by nonlinear least-squares technique. The area under the concentration-time curve (AUC) was calculated by the trapezoidal rule. Pharmacokinetic exposures for doses not directly measured in the PK study were estimated by linear extrapolation for higher and lower dose levels and by interpolation for dose levels within the dose range studied given the linear PK results.

Treatment Efficacy and Pharmacodynamic Target Determination of CD101.

Neutropenic mice were infected with one of 10 Candida strains as described above. The dosing regimens were chosen to vary the magnitude of the 24-h AUC/MIC index and to attempt to produce treatment effects that ranged from no effect to a maximal effect. Five dose levels that varied from 0.25 to 64 mg/kg were administered once in a 0.2-ml volume by IP route for the 168 h study period. Due to enhanced effect against single isolate, additional studies at 0.0156 and 0.0625 mg/kg was examined for C. glabrata 5592. Groups of three mice were used for each dosing regimen and control group. At the end of the treatment period (168 h), the mice were euthanized, and their kidneys were immediately processed for CFU determination as described above.

Data Analysis.

A sigmoid dose-effect (Hill) model was used to measure the in vivo potency of CD101. The efficacy endpoints included the dose level required to produce a 24 h net static effect (no change in organism burden compared to that at the start of therapy) and the dose required to achieve a 1-log₁₀ reduction in colony counts (relative to the burden at the start of therapy), when achieved. The maximum response (Eₘₐₓ) was measured as the difference in the number of CFU/kidney relative to that of the untreated control animals. The doses associated with the stasis and 1-log₁₀ endpoint for each strain was calculated using the equation: log₁₀ D=[log₁₀ (E/(Eₘₐₓ−E))/N]+log₁₀ ED₅₀, where D is the drug dose, E is the control growth in untreated animals, Eₘₐₓ, is the maximal effect, N is the slope of the dose-response relationship, and ED₅₀ is the dose needed to achieve 50% of the maximal effect. The associated AUC/MIC targets were then calculated for each strain. We used the PK/PD index AUC/MIC in this study as this has been shown to be associated with treatment efficacy in previous in vivo studies with the echinocandins. The calculations were performed using both total and free drug concentrations. The coefficient of determination (R²) was used to estimate the variance that might be due to regression with the PK/PD index. Kruskal-Wallis one-way analysis of variance (ANOVA) was used to determine if the differences in PK/PD targets were significant between the species.

### Results

In Vitro Susceptibility Studies.

The MICs of CD101 for the selected strains is shown in Table 15. Additionally, given the similarity of CD101 to anidulafungin, the comparative MICs to anidulafungin are shown. Of note, the strains included those with known resistance (C. glabrata 10956 is echinocandin resistant secondary to FKS mutation FKS2_HS1_F659V) or reduced susceptibility (C. glabrata 35315) to echinocandins. Overall, the CD101 MIC varied by 32-fold for all strains.

Pharmacokinetics.

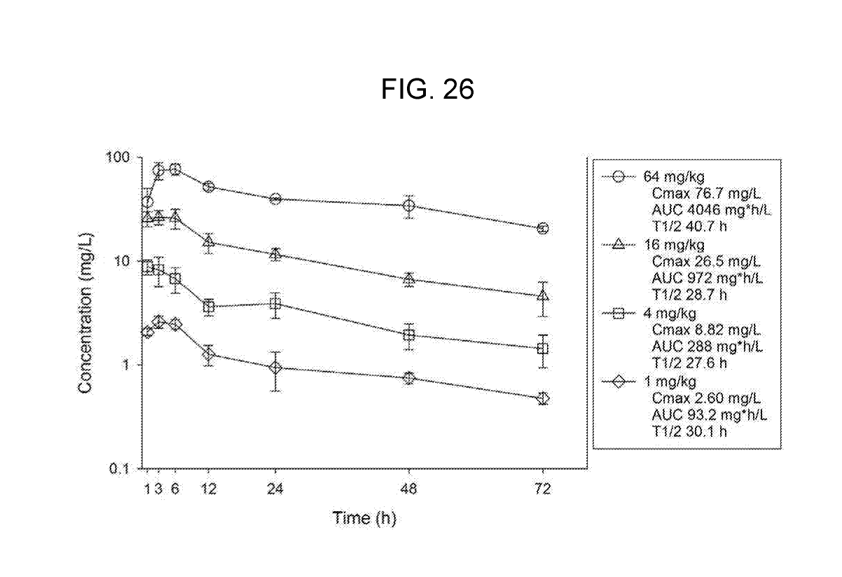

The time course of plasma concentrations of CD101 in mice after intraperitoneal doses of 1, 4, 16, and 64 mg/kg are shown in FIG. 26. Peak (Cₘₐₓ) levels ranged from 2.6-76.7 mg/L, AUC_{0-∞}93.2-40464 mg*h/L, and elimination half-life ranged from 28-41 h. The AUC_{0-∞} was linear (R²=1) over the dose range. Protein binding was 99.2%.

Treatment Efficacy and Pharmacodynamic Target Determination of CD101.

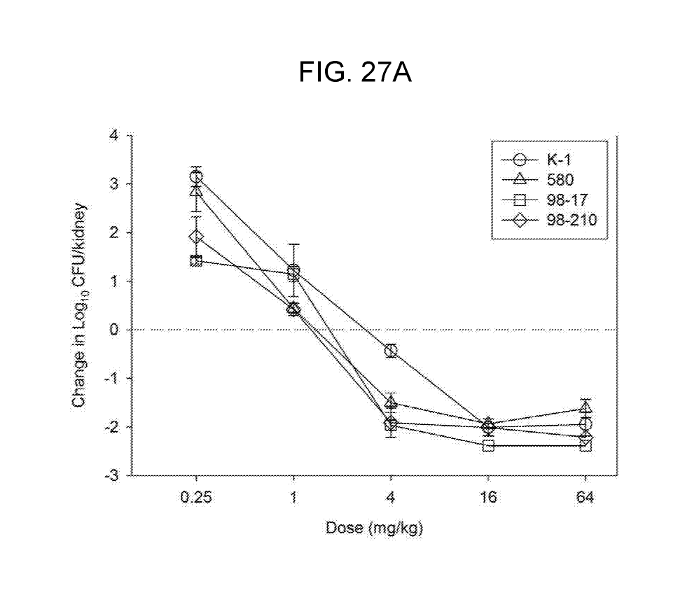

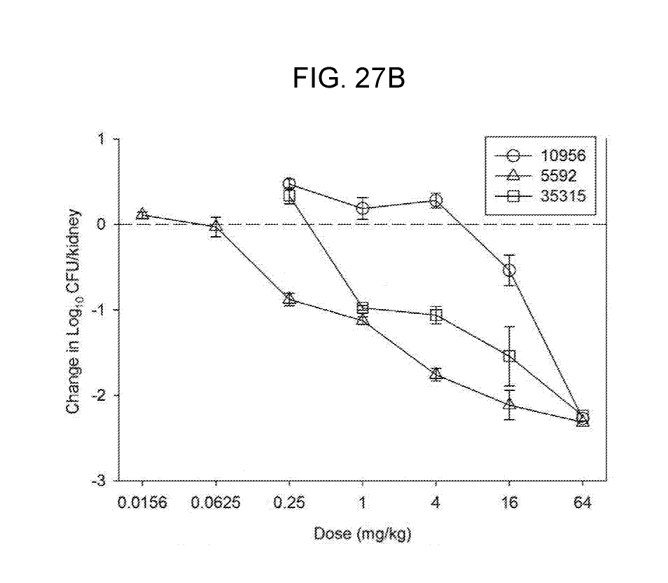

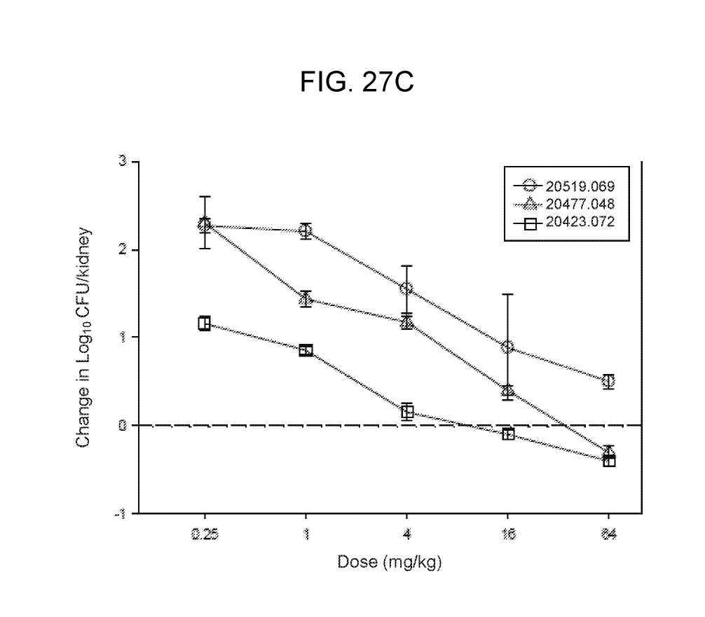

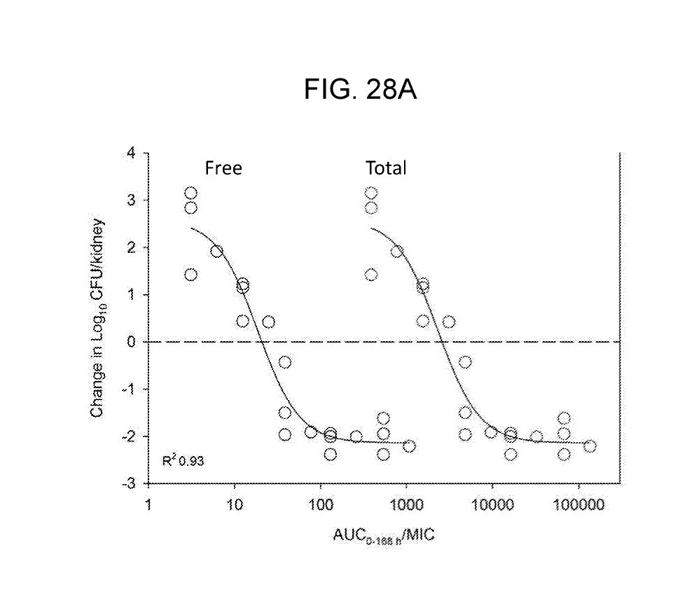

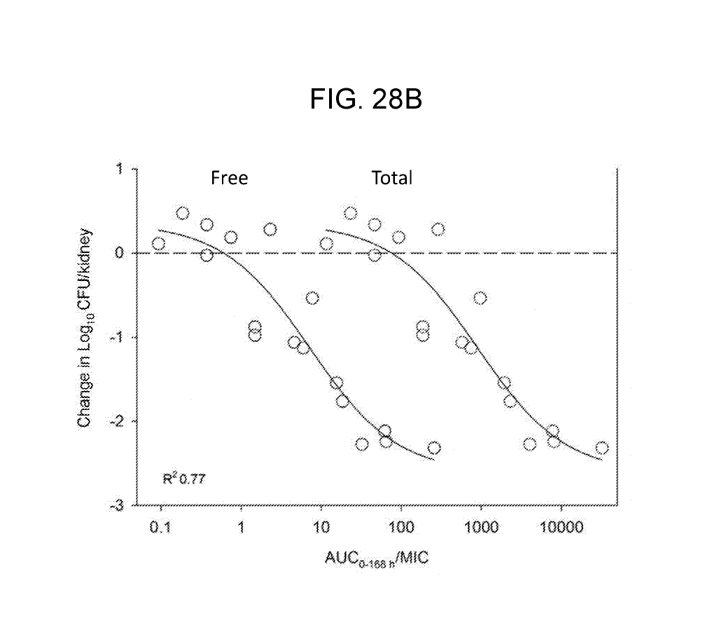

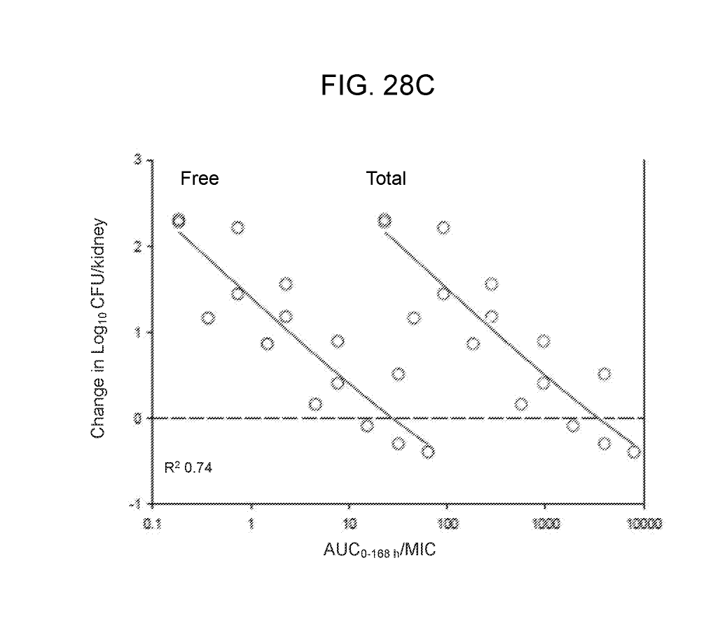

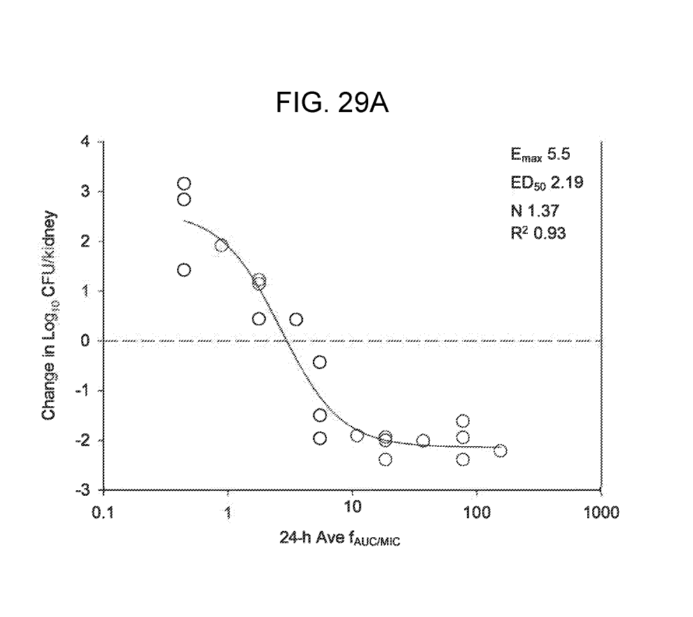

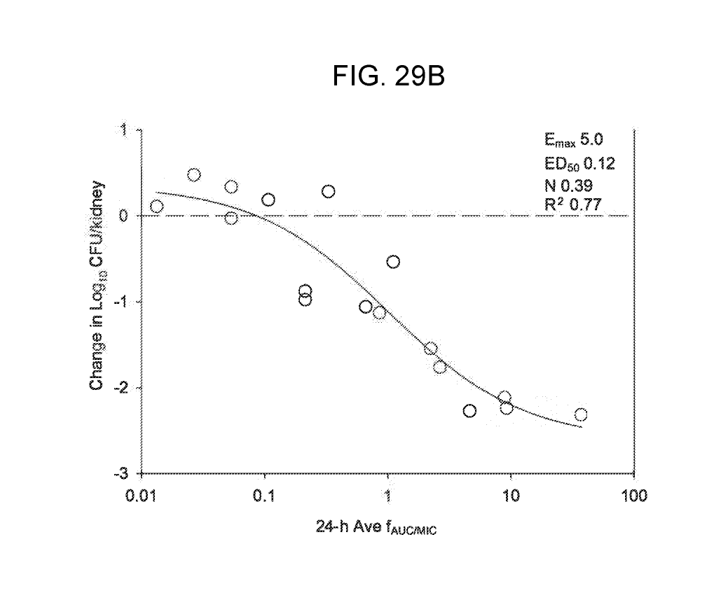

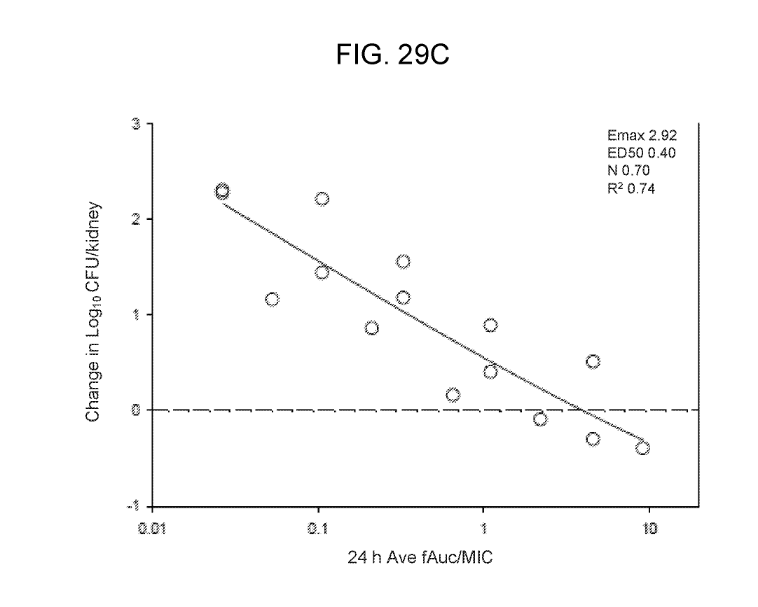

At the start of therapy, mice had 4.2±0.2 log₁₀ CFU/kidney and burden increased in untreated controls to 7.2±0.6 login CFU/kidney. The in vivo dose-response curves for each group of organisms is shown in FIGS. 27A-27C. Dose-dependent activity was observed with each group with marked potency at high doses against C. albicans and C. glabrata as a >2-log₁₀ kill was observed against a number of strains. Potency was less pronounced against C. parapsilosis, although based on the dose-response curve we speculate higher doses would have achieved similar activity for this species. The relationship between the PK/PD parameter AUC/MIC over the treatment period (168 h) and treatment effect is shown in FIGS. 28A-28C. Both free and total drug concentrations are shown with the best fit-line based on the Hill equation. The coefficients of determination (R²) were strong ranging from 0.74-0.93. Finally, shown in FIGS. 29A-29C is the average 24 h free drug AUC/MIC in order to augment comparison with previous echinocandin studies which have focused on 24 h PK/PD targets.

The doses necessary to achieve net stasis and 1-log₁₀ kill (when endpoint was achieved) are shown in Table 16. The corresponding total and free drug AUC/MIC values for the stasis and

1-log₁₀ kill endpoints are shown for the total experiment duration of 168 h (7 d). As above, also shown in the table is the average 24 h free drug AUC/MIC targets to allow for comparison to other echinocandin studies in this model. Stasis was achieved against all but a single strain and 1-log₁₀ kill was achieved against all C. albicans and C. glabrata but none of the C. parapsilosis strains. The median stasis free drug AUC₀₋₁₆₈/MIC targets for each organism group was: C. albicans 20.5, C. glabrata 0.5, and C. parapsilosis 18.2 (only two strains achieved the endpoint). The median stasis 24 h free drug AUC/MIC targets were: C. albicans 2.92, C. glabrata 0.07, and C. parapsilosis 2.61. The PK/PD targets for 1-log₁₀ kill endpoint were 2-4 fold higher than stasis targets indicating a relatively steep exposure-response relationship.

TABLE 16
Static and 1-log kill doses and associated AUC/MIC values in the neutropenic disseminated candidiasis model.

| | | | | Stasis | Stasis | Stasis 24 h | 1 log | 1 log kill | 1 log kill | 1 log kill |
|---|---|---|---|---|---|---|---|---|---|---|
| | | | Static | total drug | free drug | Ave | kill | total drug | free drug | 24 h Ave |
| | | MIC | dose | AUC₀₋₁₆₈ₕ/ | AUC₀₋₁₆₈ₕ/ | free drug | dose | AUC₀₋₁₆₈ₕ/ | AUC₀₋₁₆₈ₕ/ | Free drug |
| Organism | Strain | (mg/L) | (mg/kg) | MIC | MIC | AUC/MIC | (mg/kg) | MIC | MIC | AUC/MIC |
| C. albicans | K-1 | 0.06 | 2.52 | 3197.16 | 25.58 | 3.65 | 5.26 | 6005.95 | 48.05 | 6.86 |
| | 580 | 0.06 | 1.20 | 1769.30 | 14.15 | 2.02 | 2.03 | 2667.21 | 21.34 | 3.05 |
| | 98-17 | 0.06 | 1.34 | 1918.43 | 15.35 | 2.19 | 2.73 | 3433.40 | 27.47 | 3.92 |
| | 98-210 | 0.03 | 1.06 | 3241.65 | 25.93 | 3.70 | 2.28 | 5875.95 | 47.01 | 6.72 |
| | Mean | | 1.53 | 2531.64 | 20.25 | 2.89 | 3.08 | 4495.63 | 35.97 | 5.14 |
| | Median | | 1.27 | 2557.79 | 20.46 | 2.92 | 2.51 | 4654.68 | 37.24 | 5.32 |
| | St Dev | | 0.67 | 796.71 | 6.37 | 0.91 | 1.49 | 1698.80 | 13.59 | 1.94 |
| C. glabrata | 10956 | 1 | 6.29 | 418.68 | 3.35 | 0.48 | 17.25 | 1052.22 | 8.42 | 1.20 |
| | 5592 | 0.125 | 0.06 | 43.16 | 0.35 | 0.05 | 0.43 | 317.50 | 2.54 | 0.36 |
| | 35315 | 0.5 | 0.34 | 62.50 | 0.50 | 0.07 | 2.39 | 367.06 | 2.94 | 0.42 |
| | Mean | | 2.23 | 174.78 | 1.40 | 0.20 | 6.69 | 578.93 | 4.63 | 0.66 |
| | Median | | 0.34 | 62.50 | 0.50 | 0.07 | 2.39 | 367.06 | 2.94 | 0.42 |
| | St Dev | | 3.52 | 211.44 | 1.69 | 0.24 | 9.20 | 410.63 | 3.29 | 0.47 |
| C. | 20519.069 | 1 | NA* | | | | NA | | | |
| parapsilosis | 20477.048 | 1 | 52.96 | 3339.42 | 26.72 | 3.82 | NA | | | |
| | 20423.072 | 0.5 | 9.62 | 1217.49 | 9.74 | 1.39 | NA | | | |

*NA, not achieved

### Discussion

In the current murine model pharmacodynamic study, we aimed to integrate the pharmacokinetic properties and in vitro potency to provide guidance on pharmacodynamic targets associated with efficacy against a clinically relevant and diverse group Candida spp. Indeed, the pharmacokinetics of CD101 were unique in that the elimination half-life in mice was prolonged (range 29-41 h). For comparison purposes, the half-life of other echinocandins in the same murine model are on average approximately 14 h. We also demonstrated promising in vitro potency against Candida spp. similar to previous larger surveillance antimicrobial susceptibility studies. Finally, we demonstrated CD101 has favorable in vivo efficacy using the murine disseminated candidiasis model with numerically lower PK/PD target exposures for most organisms compared to other echinocandins. For example, the median stasis 24 h free drug AUC/MIC against C. albicans was 2.92. This is 5- to 10-fold lower than caspofungin, micafungin, and anidulafungin targets against this species. An even larger difference was demonstrated for C. glabrata where CD101 free AUC/MIC targets were >10-fold lower than the three comparator echinocandins. C. parapsilosis PK/PD target analysis was limited in the current study as only two strains were evaluable for the stasis target endpoint, but here too CD101 free AUC/MIC targets were numerically lower than the three comparator echinocandins. It is important to note that due to the prolonged half-life, mice were protected from organism growth and disease for a neutropenic duration of 7 days. Taken together, the study demonstrates that CD101 is a potential valuable addition to the antifungal armamentarium given its unique pharmacokinetic properties and in vivo efficacy.

An important consideration in translating pre-clinical PK/PD target models to clinical medicine is to examine the targets identified in the context of human pharmacokinetics and surveillance susceptibility ranges. Pharmacokinetic study of CD101 in humans demonstrated a free drug AUC₀₋₁₆₈ of 30.2 mg*h/L for a 400 mg dose and 15.4 mg*h/L for a 200 mg dose. This would translate into an average 24 h AUC of approximately 4.3 and 2.2 mg*h/L, respectively, over a 7 day period. Thus, if a patient were to receive 400 mg of CD101 on Day 1 followed by a 200 mg on Day 8 to complete two weeks of therapy, the stasis target would be expected to be achieved against all C. albicans and C. parapsilosis isolates with MIC 1 mg/L, and against all C. glabrata with MIC 16 mg/L. The potency against C. glabrata, including an FKS mutant strain included in this study, deserves particular attention to further study given the rise of echinocandin resistance within this species. Overall, the data suggests CD101 exposures in humans would be expected to meet or exceed the stasis targets identified in this study for nearly all wild-type isolates for the examined species.

In summary, CD101 is a promising, novel echinocandin in development with advantageous pharmacokinetic properties allowing for once weekly dosing strategies, which would mitigate risks to patients as well as conservation of health care resources and potentially lower expenditures. CD101 has demonstrable in vitro and in vivo potency that is either equivalent or improved upon comparator echinocandins, especially in regards to C. glabrata. Single doses provided 7 days of potent antifungal activity in a well-established immunocompromised disseminated candidiasis model. Importantly, the PK/PD targets identified suggest that current studies of intermittent dosing strategies (i.e. once weekly infusions) of CD101 are likely to be efficacious in humans against the majority of C. albicans, C. glabrata, C. parapsilosis strains. The studies indicate continued clinical evaluation and development for the treatment and prevention of invasive candidiasis as well as other potential fungal infections should be pursued.

### Example 13: Efficacy of CD101 in a Murine Model of Pulmonary Aspergillosis

This study assessed the antifungal efficacy of CD101 by intraperitoneal administration in a murine model of pulmonary aspergillosis caused by Aspergillus fumigatus (strain AF293) compared to micafungin. The primary objective of the study was to compare survival between the treatment groups.

### Methods

Animal Strain and Housing.

Mice used in these studies were supplied by Charles River (Margate UK) and were specific pathogen free. The strain of mice used was ICR (also known as CD1 Mice) which is a well characterized outbred murine strain. Mice (male) were 11-15 g on receipt at our facility and were allowed to acclimatize for at least seven days.

Immunosuppression.

Mice were immunosuppressed on Day −4 with 150 mg/kg cyclophosphamide IP, and on Day −1 with 150 mg/kg cyclophosphamide IP and 175 mg/kg cortisone acetate SC. To prevent bacterial infection due to the immunosuppression mice were given 50 mg/kg/day ceftazidime.

Preparation of Organism and Infection.

A. fumigatus strain AF293 inoculum was prepared from spore cultures grown on Sabouraud Dextrose agar (SAB) containing 50 μg/mL chloramphenicol (SABC) in vented tissue culture flasks. Following incubation for 7-10 days at 30° C., spore cultures were washed in sterile phosphate buffered saline (PBS) containing 0.05% Tween 80. Spore count was determined using a haemocytometer and spores were diluted in PBS to ˜6.9×10⁶ CFU/mL. Inoculum concentration was confirmed by quantitative culture onto SABC agar. Neutropenic mice lungs were infected with 0.04 mL (0.02 mL/nares) of ˜6.9×10⁶ CFU/mL (˜2.8×10⁵ cfu/mouse) of A. fumigatus strain AF293 by intranasal (IN) instillation under temporary 2.5% isoflurane induced anesthesia.

Preparation of Test Articles.

Micafungin was provided as a 50 mg vial (Lot 02323002, expiry August 2017) and was prepared as per manufacturer instructions by adding 5 mL saline for injection (SFI) directly into the vial to make a 10 mg/mL stock solution. This solution was then diluted further in SFI to a working concentration of 0.2 mg/mL. The compound was administered IP at 10 mL/kg to achieve a 2 mg/kg dose. It was prepared fresh once and stored at 4° C. between doses.

Vehicle and CD101 diluent was 10% DMSO/1% Tween 20 in SFI: 1 mL of Tween 20 was added to 10 mL DMSO and the gently mixed and SFI added to a final volume of 100 mL. This was filter sterilised and maintained at room temperature before use for dosing or formulating CD101. The vehicle was administered IP at 10 mL/kg.

Test article CD101 stock was prepared at 2 mg/mL in 10% DMSO/1% Tween 20 diluent. A clear non particulate solution was obtained following gentle mixing. The stock was kept at 4° C. until required. Study doses of 5 mg/kg (0.5 mg/mL) and 10 mg/kg (1 mg/mL) were prepared from the 2 mg/mL stock as required by diluting in 10% DMSO/1% Tween 20 diluent. The 2 mg/mL stock was used undiluted for the 20 mg/kg study dose. All doses were administered IP at 10 mL/kg.

Treatment.

For this study, treatments were initiated on day five pre-infection according to treatment groups outlined in Table 17. A total of 78 mice (six mice per treatment group) were used in the study.

TABLE 17
Murine model of pulmonary aspergillosis treatment groups

| | | | | | Conc. | | | | | |
|---|---|---|---|---|---|---|---|---|---|---|
| | Test | | | Dosing | mg/mL | | | Total | Mice | End of |
| Group | Article | Route | Schedule | Day | ml/kg | mg/kg | Dosage | Dose | (ICR) | study |
| 1 | Vehicle | IP | Single | −5 | — | — | — | — | 6 | 10 |
| 2 | Micafungin | IP | Single | 0* | 0.2 | 10 | 2 | 2 | 6 | 10 |
| 3 | Micafungin | IP | Single | −1 | 0.2 | 10 | 2 | 2 | 6 | 10 |
| 4 | CD101 | IP | Single | 0* | 0.5 | 10 | 5 | 5 | 6 | 10 |
| 5 | CD101 | IP | Single | −1 | 0.5 | 10 | 5 | 5 | 6 | 10 |
| 6 | CD101 | IP | Single | −3 | 0.5 | 10 | 5 | 5 | 6 | 10 |
| 7 | CD101 | IP | Single | −5 | 0.5 | 10 | 5 | 5 | 6 | 10 |
| 8 | CD101 | IP | Single | −1 | 1 | 10 | 10 | 10 | 6 | 10 |
| 9 | CD101 | IP | Single | −3 | 1 | 10 | 10 | 10 | 6 | 10 |
| 10 | CD101 | IP | Single | −5 | 1 | 10 | 10 | 10 | 6 | 10 |
| 11 | CD101 | IP | Single | −1 | 2 | 10 | 20 | 20 | 6 | 10 |
| 12 | CD101 | IP | Single | −3 | 2 | 10 | 20 | 20 | 6 | 10 |
| 13 | CD101 | IP | Single | −5 | 2 | 10 | 20 | 20 | 6 | 10 |

*1 h post infection

General Health Monitoring.

The mice were monitored at a frequency appropriate for their clinical condition. Mouse weights were recorded at least once daily both to ensure animals remained within ethical limits and to monitor efficacy of treatment.

Endpoints.

The primary endpoint for this study was survival within agreed ethical limits (>20% weight loss, severe hypothermia <34° C., inability to reach food or drink, severe hunching). Mice were monitored by daily weight measurements with observations as frequently as clinical condition required. Mice presenting with severe clinical deterioration were humanely euthanized using an overdose of pentobarbitone administered by IP injection following clinical assessment and the time of death was recorded. Animal carcasses were stored at −20° C. for assessment of burden. Ten days post infection all surviving animals were weighed and had their clinical condition assessed prior to being euthanized. Final survival numbers were recorded and analyzed as described below and carcasses frozen at −20° C. prior to further processing.

A secondary endpoint for the study was terminal lung tissue burden. Immediately following confirmation of death, carcasses were frozen at −20° C. prior to tissue dissection and processing. The frozen carcasses were thawed at room temperature and the lungs removed and placed into pre-weighed bead-beating tubes containing 2 mL of PBS and subjected to mechanical disruption. Organ homogenates were diluted further in PBS and quantitatively cultured for A. fumigatus onto SABC and incubated at 30° C. for 24-48 hours. In addition, a 300 μL aliquot of the undiluted lung tissue homogenate was stored at −80° C. for possible optional assessment of burden by qPCR.

Statistical Analysis.

Data were analyzed using StatsDirect software (version 2.7.8). Survival data were analyzed using the Kaplan Meier and Log-Rank and Wilcoxon tests (using the Peto-Prentice weighting method).

### Results

The aim of this study was to determine the in vivo efficacy of CD101 in a murine model of pulmonary aspergillosis. The design of this study is summarized in Table 17. All treatments were well tolerated with no adverse signs observed.

Body Weights.

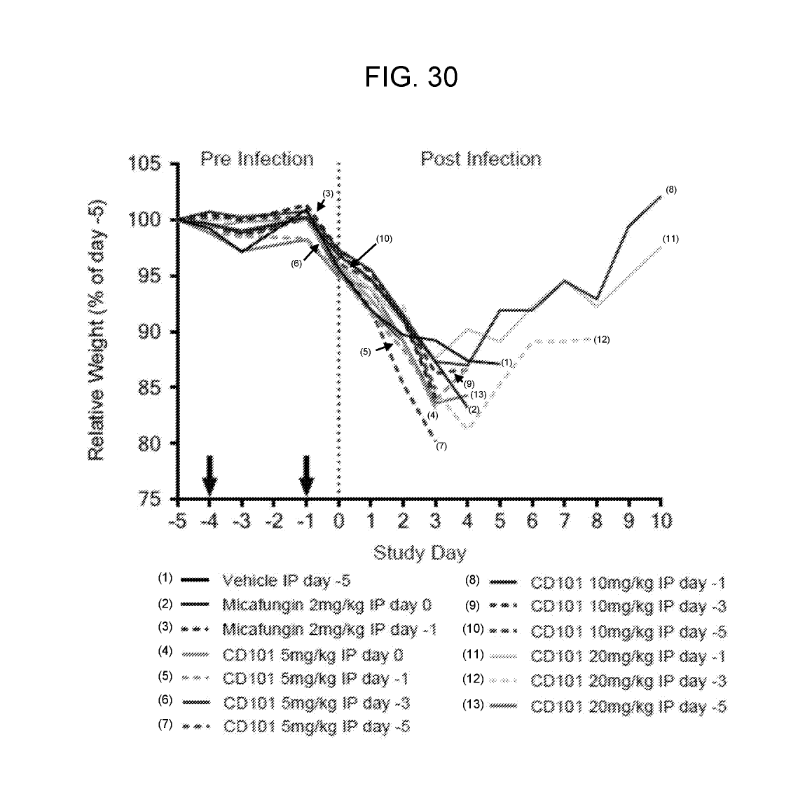

Animal weights following infection with A. fumigatus strain AF293 are shown in FIG. 30. Animal weights are shown relative to the weight on Day 5 pre-infection (first treatment time). Weights remained stable up to Day −1 pre-infection. Mice from all treatment groups lost weight following the immunosuppression on Day −1. The weight loss continued after the infection in almost all treatments groups except mice treated with CD101 at 10 mg/kg on Day −1 and CD101 at 20 mg/kg on Day −3 and Day −1 from three days post infection.

Survival.

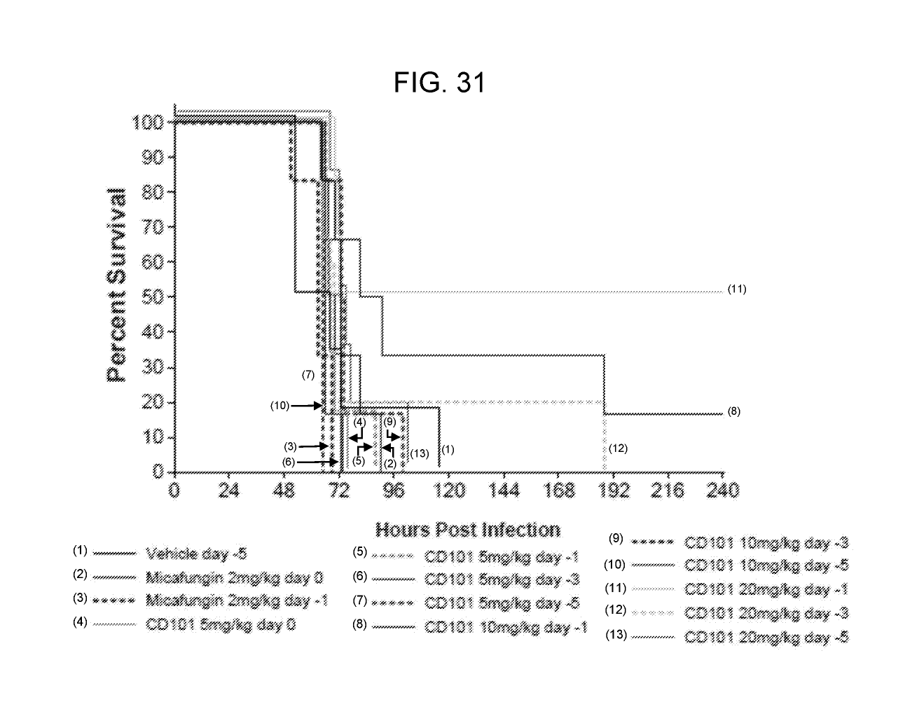

The median and mean survival for the various treatments are shown in Table 18 and the survival plots for all treatment groups are shown in FIG. 31. Statistical outcomes from the Log-Rank and Wilcoxon test are shown in Table 19.

TABLE 18
Mean and median survival per treatment group

| | Median Survival | Mean Survival |
|---|---|---|
| Treatment | (Hours) | (Hours) |
| Vehicle IP Day −5 | 52.5 | 69.0 |
| Micafungin 2 mg/kg IP Day 0 | 73.3 | 75.3 |
| Micafungin 2 mg/kg IP Day −1 | 64.7 | 64.7 |
| CD101 5 mg/kg IP Day 0 | 64.6 | 67.8 |
| CD101 5 mg/kg IP Day −1 | 67.6 | 72.1 |
| CD101 5 mg/kg IP Day −3 | 67.7 | 70.0 |
| CD101 5 mg/kg IP Day −5 | 65.3 | 65.3 |
| CD101 10 mg/kg IP Day −1 | 81.0 | 121.8 |
| CD101 10 mg/kg IP Day −3 | 73.4 | 76.9 |
| CD101 10 mg/kg IP Day −5 | 65.7 | 67.2 |
| CD101 20 mg/kg IP Day −1 | 72.3 | 155.6 |
| CD101 20 mg/kg IP Day −3 | 69.8 | 91.9 |
| CD101 20 mg/kg IP Day −5 | 69.8 | 75.5 |

A robust survival model of pulmonary aspergillosis infection with A. fumigatus strain AF293 was established. Vehicle treated mice started to succumb to the infection ˜48 h post infection and all had succumbed to the infection by Day 5 post infection resulting in a mean survival time of 69 h post infection. The study was terminated 10 days post infection as most mice had succumbed to the infection.

Survival in animal groups treated with CD101 was compared against groups treated with the comparator micafungin at the same time pre- or post-infection. Groups treated with 10 mg/kg and 20 mg/kg CD101 one day before infection had statistically greater survival compared to groups treated with micafungin one day before infection (Table 19).

TABLE 19
Log-rank and Wilcoxon test output for different comparisons

| | Log- | Peto- |
|---|---|---|
| Comparison | Rank | Prentice |
| Vehicle vs. Micafungin 2mpk Day 0 | NS | NS |
| Vehicle vs. Micafungin 2mpk Day −1 | NS | NS |
| Vehicle vs. CD101 5mpk Day 0 | NS | NS |
| Vehicle vs. CD101 5mpk Day −1 | NS | NS |
| Vehicle vs. CD101 5mpk Day −3 | NS | NS |
| Vehicle vs. CD101 5mpk Day −5 | NS | NS |
| Vehicle vs. CD101 10mpk Day −1 | NS | NS |
| Vehicle vs. CD101 10mpk Day −3 | NS | NS |
| Vehicle vs. CD101 10mpk Day −5 | NS | NS |
| Vehicle vs. CD101 20mpk Day −1 | NS (0.0533) | 0.0465 |
| Vehicle vs. CD101 20mpk Day −3 | NS | NS |
| Vehicle vs. CD101 20mpk Day −5 | NS | NS |
| Micafungin 2mpk Day 0 vs. CD101 5mpk Day 0 | NS | NS |
| Micafungin 2mpk Day −1 vs. CD101 5mpk | NS | 0.0467 |
| Day −1 | | |
| Micafungin 2mpk Day −1 vs. CD101 10mpk | 0.0201 | 0.0191 |
| Day −1 | | |
| Micafungin 2mpk Day −1 vs. CD101 20mpk | 0.0047 | 0.0067 |
| Day −1 | | |
| CD101 5mpk Day −1 vs. CD101 10mpk Day −1 | NS | NS |
| CD101 5mpk Day −1 vs. CD101 20mpk Day −1 | NS | NS |
| CD101 5mpk Day −3 vs. CD101 10mpk Day −3 | NS | NS |
| CD101 5mpk Day −3 vs. CD101 20mpk Day −3 | NS | NS |
| CD101 5mpk Day −5 vs. CD101 10mpk Day −5 | 0.0009 | 0.0015 |
| CD101 5mpk Day −5 vs. CD101 20mpk Day −5 | 0.0009 | 0.0015 |
| CD101 5mpk Day 0 vs. CD101 5mpk Day −1 | NS | NS |
| CD101 5mpk Day 0 vs. CD101 5mpk Day −3 | NS | NS |
| CD101 5mpk Day 0 vs. CD101 5mpk Day −5 | NS | NS |
| CD101 5mpk Day −1 vs. CD101 5mpk Day −3 | NS | NS |
| CD101 5mpk Day −1 vs. CD101 5mpk Day −5 | NS | NS |
| CD101 5mpk Day −3 vs. CD101 5mpk Day −5 | 0.0183 | 0.0426 |
| CD101 10mpk Day −1 vs. CD101 10mpk Day −3 | NS | NS |
| CD101 10mpk Day −1 vs. CD101 10mpk Day −5 | NS | NS |
| CD101 10mpk Day −3 vs. CD101 10mpk Day −5 | NS | NS |
| CD101 20mpk Day −1 vs. CD101 20mpk Day −3 | 0.0387 | 0.033 |
| CD101 20mpk Day −1 vs. CD101 20mpk Day −5 | NS | NS |
| CD101 20mpk Day −3 vs. CD101 20mpk Day −5 | NS | NS |

NS—not significant

### Lung Burden.

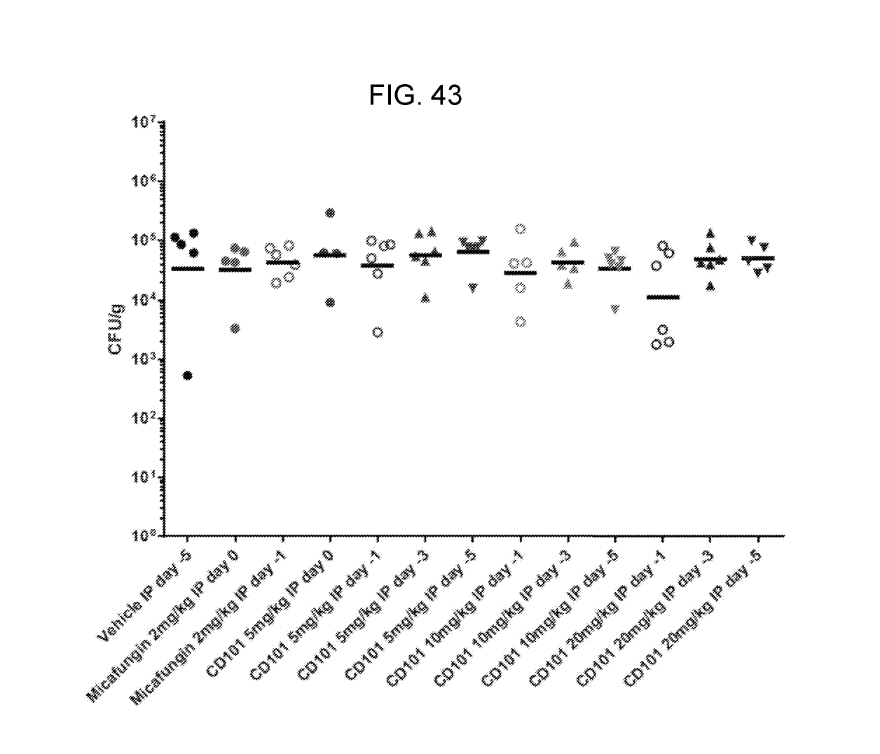

Terminal lung burden are shown in Table 20 and FIG. 43.

TABLE 20
Lung burden

| | | | | Log₁₀ |
|---|---|---|---|---|
| | Group | | Log₁₀ Group | reduction |
| | Geometric | Standard | Geometric | from vehicle |
| | mean | Deviation | mean | control |
| Treatment | (CFU/g) | (CFU/g) | (CFU/g) | (CFU/g) |
| Vehicle | 5.93 × 10³ | 5.67 × 10⁴ | 3.77 | 0.00 |
| IP Day −5 | | | | |
| Micafungin 2 mg/kg | 1.08 × 10⁴ | 3.10 × 10⁴ | 4.03 | −0.26 |
| IP Day 0 | | | | |
| Micafungin 2 mg/kg | 4.34 x 10⁴ | 2.65 × 10⁴ | 4.64 | −0.86 |
| IP Day −1 | | | | |
| CD1015 mg/kg | 5.65 × 10⁴ | 1.28 × 10⁵ | 4.75 | −0.98 |
| IP Day 0 | | | | |
| CD101 5 mg/kg | 3.75 × 10⁴ | 3.75 × 10⁴ | 4.57 | −0.80 |
| IP Day −1 | | | | |
| CD101 5 mg/kg | 5.71 × 10⁴ | 5.21 × 10⁴ | 4.76 | −0.98 |
| IP Day −3 | | | | |
| CD101 5 mg/kg | 6.36 × 10⁴ | 2.99 × 10⁴ | 4.80 | −1.03 |
| IP Day −5 | | | | |
| CD101 10 mg/kg | 5.17 × 10³ | 5.89 × 10⁴ | 3.71 | 0.06 |
| IP Day −1 | | | | |
| CD101 10 mg/kg | 7.34 × 10³ | 3.39 × 10⁴ | 3.87 | −0.09 |
| IP Day −3 | | | | |
| CD101 10 mg/kg | 3.40 × 10⁴ | 1.97 × 10⁴ | 4.53 | −0.76 |
| IP Day −5 | | | | |
| CD101 20 mg/kg | 1.12 × 10⁴ | 3.46 × 10⁴ | 4.05 | −0.28 |
| IP Day −1 | | | | |
| CD101 20 mg/kg | 4.92 × 10⁴ | 4.12 × 10⁴ | 4.69 | −0.92 |
| IP Day −3 | | | | |
| CD101 20 mg/kg | 8.35 × 10³ | 3.58 × 10⁴ | 3.92 | −0.15 |
| IP Day −5 | | | | |

### Conclusions

In this model of pulmonary aspergillosis, mice developed robust infection with ˜80% vehicle treated mice succumbing to the infection by Day 4 post infection and 100% mice by Day 5 post infection.

Single dose CD101 treatment one day pre-infection at 10 mg/kg or 20 mg/kg (the CD101 human dose (400 mg) AUC equivalent) resulted in statistically greater survival compared to comparator 2 mg/kg (the micafungin human dose (50 mg) AUC equivalent) micafungin treatment one day pre-infection.

### Example 14: CD101 Prophylactic Dose Rationale for Prevention of Aspergillus, Candida, and Pneumocystis Infections

Clinical pharmacokinetics of CD101 were compared to measures of nonclinical in vitro susceptibility and in vivo efficacy to guide dose selection for prevention of fungal infections.

### Methods

The protein binding of CD101 to mouse and human plasma proteins was measured by ultracentrifugation, where free compound is separated from protein-bound compound after 2.5 hr at 37° C. by sedimentation using high centrifugal force. Concentrations in plasma ranging from 7 to 60 μg/mL were tested and resulting samples were analyzed by LC-MS/MS.

The in vitro activity of CD101 was previously evaluated as part of the SENTRY international surveillance program using CLSI broth microdilution methodology (M38-A2, M27-A3; Pfaller, et al 2017). CD101 demonstrated potent activity against Aspergillus fumigatus (MEC₉₀=0.015 μg/mL) and Candida albicans (MIC₉₀=0.06 μg/mL) clinical isolates. These susceptibility data were then compared graphically to CD101 plasma concentrations from studies in healthy adults, adjusted for plasma protein binding. Nonclinical efficacy in a murine model of Pneumocystis pneumonia were also considered to evaluate CD101 doses for clinical investigation of antifungal prophylaxis.

### Results

In the protein binding study, across the concentrations tested, the percent of bound CD101 ranged from 96.4% to 98.0% with a mean of 97.4% in human plasma, whereas the percent of bound CD101 ranged from 99.2% to 99.3% with a mean of 99.2% in mouse plasma.

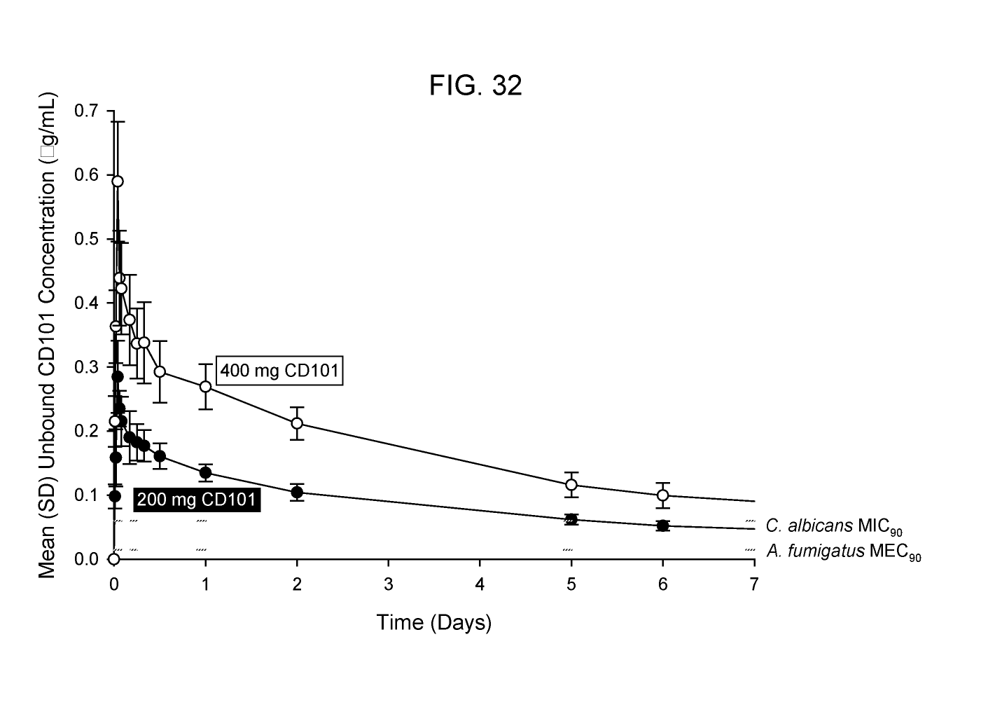

Mean unbound CD101 plasma concentrations in Phase 1 subjects following a single dose of 400 mg were above the MIC₉₀ for C. albicans for seven days, and CD101 plasma concentrations for both 400 mg and 200 mg were above the MEC₉₀ for A. fumigatus for 7 days (FIG. 32). Although standard in vitro MIC testing is not possible for Pneumocystis spp., CD101 prevented Pneumocystis pneumonia in mice at human equivalent doses of <50 mg, with results similar to standard of care (trimethoprim/sulfamethoxazole). A dose of 400 mg of CD101 appears sufficient for prevention of fungal infections, from the very first dose. Given accumulation of approximately 30% to 55% with repeat dose administration, and the fact that CD101 is fungicidal against Candida spp., CD101 at 200 mg may also be effective for fungal prophylaxis.

### Example 15: In Vitro Activity and In Vivo Tissue Distribution of CD101

The in vitro activity of CD101 was evaluated against 153 A. fumigatus clinical isolates collected as part of the 2014 and 2015 JMI international SENTRY surveillance program. Susceptibility was determined by measuring the minimal effective concentration (MEC) values in accordance with CLSI broth microdilution guidelines (M38-A2). In vivo tissue distribution of CD101 in SD rats (N=3/time up to 5 d) after a 5 mg/kg IV CD101 dose. Plasma/tissue concentrations were measured by LC-MS/MS.

CD101 demonstrated potent in vitro activity against clinical A. fumigatus isolates with MEC50, MEC90 and MEC range values of 0.015, 0.015, and ≤0.0078-0.03 μg/mL, respectively.

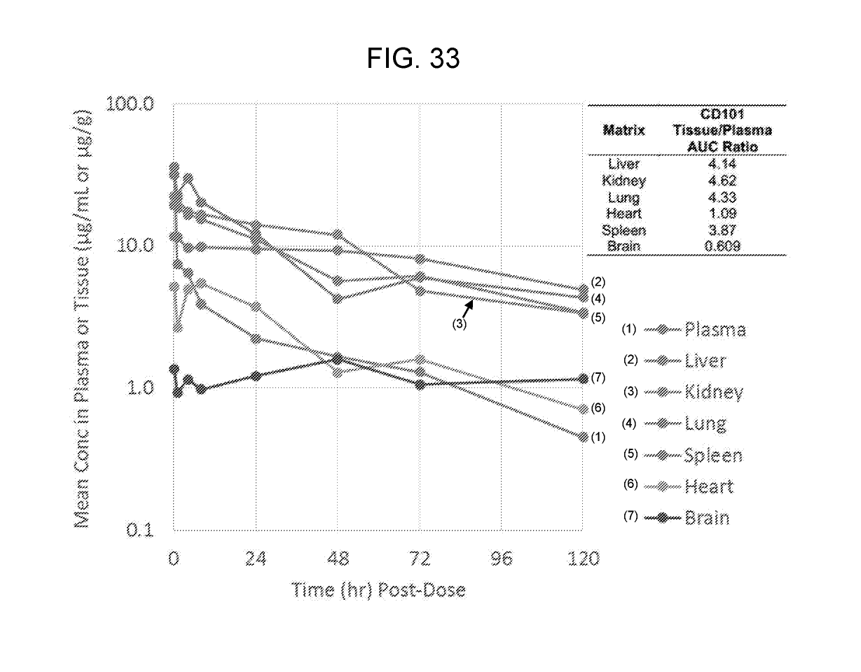

In vivo, CD101 tissue/plasma exposure ratios (˜4) were comparable among the major organs (liver, kidney, lung, spleen) suggesting efficient penetration. Also, longer tissue residence times were observed as t_{1/2} of CD101 in all tissues (40-77 h) studied were longer compared with plasma (39 h) (FIG. 33).

### Example 16: A. fumigatus (ATCC 13073) Disseminated Infection of Neutropenic ICR Mice: CD101 Prophylactic Efficacy

The study objective was to evaluate the efficacy of the test article, CD101, as prophylaxis in the Aspergillus fumigatus (ATCC 13073) disseminated infection model with neutropenic ICR mice.

### Methods

Inoculum Preparation.

A. fumigatus (ATCC 13073) growth was taken from 96 h potato dextrose agar (PDA) and re-suspended in 0.1% Tween 20. The culture was resuspended in 1 mL cold PBS (>1.0×10⁸ CFU/mL, OD620 2.3-2.8). The culture was then diluted in PBS to final cellular densities of 2.0×10⁵ CFU/mL. The actual colony counts were determined by plating dilutions on PDA plates to confirm inoculation concentration. The actual inoculum count was 1.85×10⁵ CFU/mL.

A. fumigatus (ATCC 13073) disseminated infection (IV). Groups of 6 female ICR mice weighing 22±2 g were used. Animals were immunosuppressed by three intraperitoneal (IP) injections of cyclophosphamide (the first at 6 mg/mouse 3 days before inoculation, the second and third at 2 mg/mouse on Day 1 then Day 4 after inoculation). On Day 0, animals were inoculated (0.1 mL/mouse) by intravenous (IV) injection into the tail vein with A. fumigatus (ATCC 13073), 1.85×10⁴ CFU per mouse. CD101 at 5, 10 and 20 mg/kg as prophylaxis was administered subcutaneously (SC) once starting 5, 3 or 1 day before inoculation. In addition, CD101 at 3 mg/kg SC and the reference, amphotericin B, at 3 mg/kg by intraperitoneal (IP) injection were administered one hour after infection (See Table 21).

Mortality was observed for 14 days. A 50 percent or more 50%) increase in the survival rate compared to the vehicle control group indicates significant anti-infective activity. The health observations including body weight, hunched posture, ruffled fur, immobility and hypothermia were recorded daily for 14 days. Animals found moribund were to be humanely sacrificed with CO₂ asphyxiation in the study.

TABLE 21
Study design

| Group | Article | Route | Schedule | Day | mg/mL | mL/kg | mg/kg | Dose | (ICR) |
|---|---|---|---|---|---|---|---|---|---|
| 1 | Vehicle | — | None | — | — | — | — | — | 6 |
| 2 | AmpB | IP | Single | 0^{a} | 0.3 | 10 | 3 | 3 | 6 |
| 3 | CD101 | SC | Single | 0^{a} | 0.5 | 10 | 5 | 5 | 6 |
| 4 | CD101 | SC | Single | −5 | 0.5 | 10 | 5 | 5 | 6 |
| 5 | CD101 | SC | Single | −3 | 0.5 | 10 | 5 | 5 | 6 |
| 6 | CD101 | SC | Single | −1 | 0.5 | 10 | 5 | 5 | 6 |
| 7 | CD101 | SC | Single | −5 | 1 | 10 | 10 | 10 | 6 |
| 8 | CD101 | SC | Single | −3 | 1 | 10 | 10 | 10 | 6 |
| 9 | CD101 | SC | Single | −1 | 1 | 10 | 10 | 10 | 6 |
| 10 | CD101 | SC | Single | −5 | 2 | 10 | 20 | 20 | 6 |
| 11 | CD101 | SC | Single | −3 | 2 | 10 | 20 | 20 | 6 |
| 12 | CD101 | SC | Single | −1 | 2 | 10 | 20 | 20 | 6 |

### Results

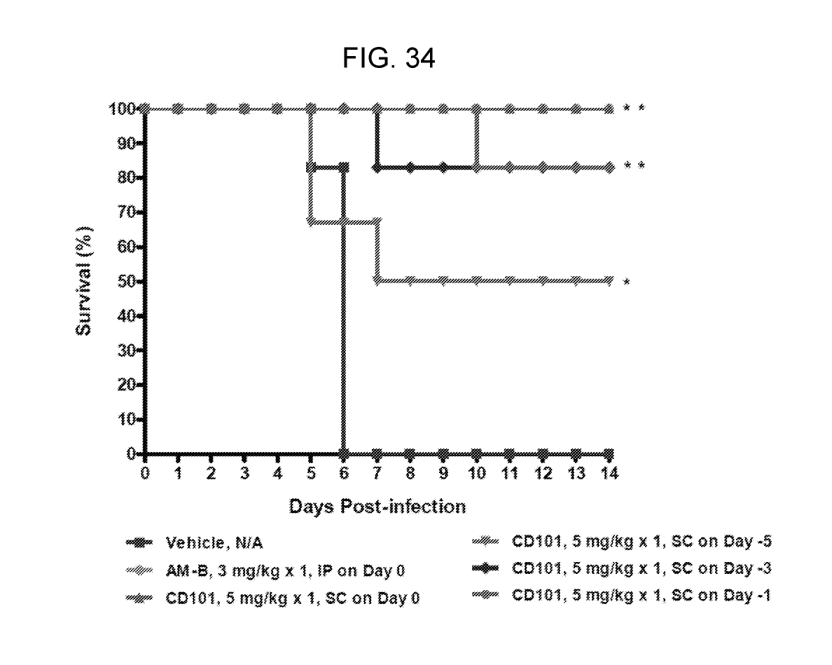

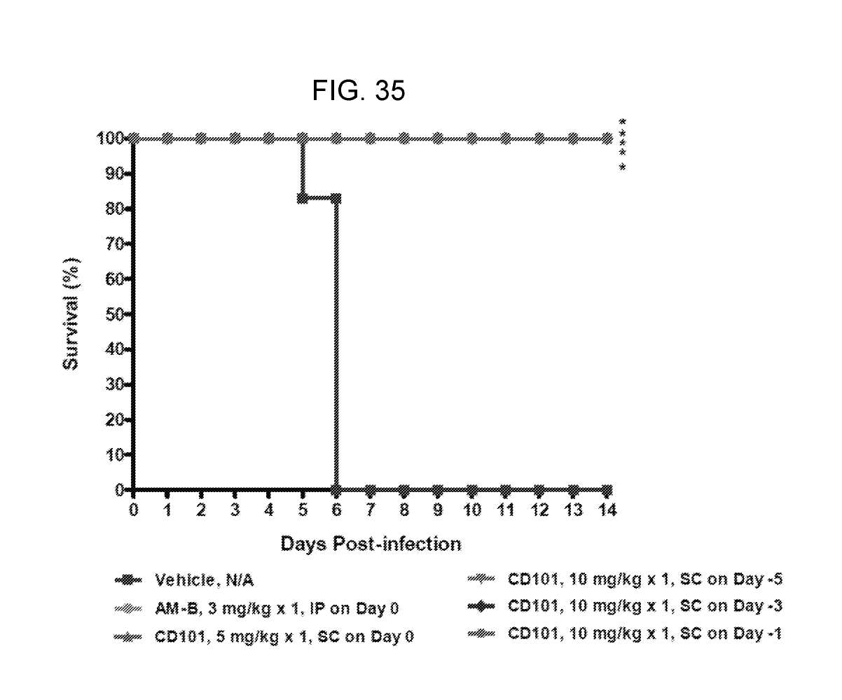

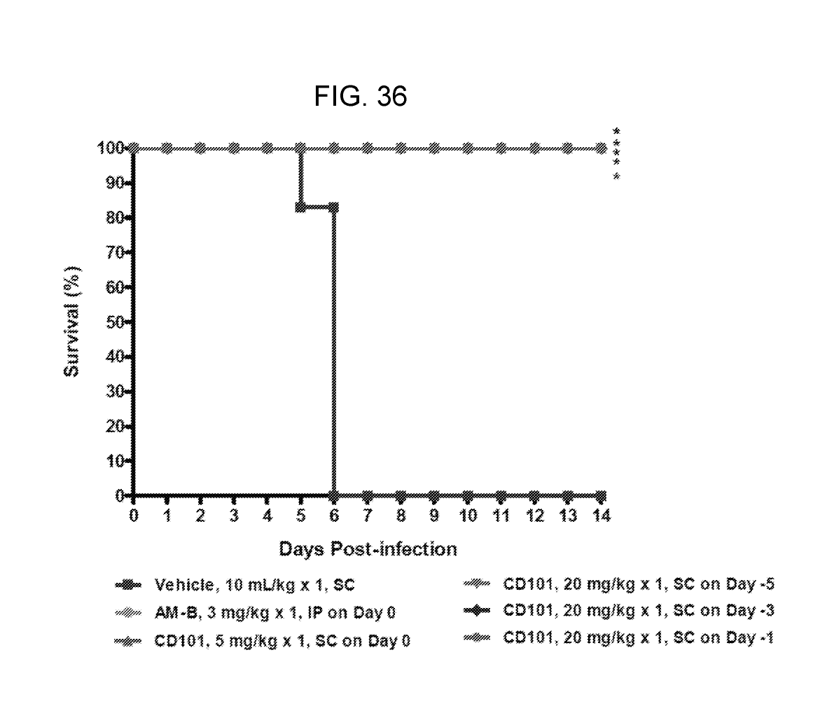

Subcutaneous administrations of CD101 at 5, 10, and 20 mg/kg on Day −5, −3 and −1 were associated with significant (≥50%) increase in the 14-day survival compared to the vehicle group (FIGS. 34-36). CD101 at 5 mg/kg SC and amphotericin B at 3 mg/kg IP administered one hour after infection were also associated with significant increase in the 14-day survival observation in the study.

In addition, the symptoms of infection including a decrease in the body weight, hunched posture, ruffled fur, immobility and hypothermia from were improved by subcutaneous administrations of CD101 at 5, 10 and 20 mg/kg on Day −5, −3 and −1 before infection.

### Example 17: CD101 Tissue and Epithelial Lining Fluid Concentrations Substantiates its Use for Prophylaxis Treatment as Evident in Mouse Disseminated and Pulmonary Apergillosis

CD101 has previously demonstrated robust efficacy in mouse antifungal models of aspergillosis. The distribution of CD101 into lung epithelial lining fluid (ELF) was studied to provide further substantiation of observed efficacy.

### Methods

CD101 (20 mg/kg) was administered by IP to 24 ICR mice. At pre-dose, 1, 3, 6, 12, 24, 48, and 72 hours post-dose, 3 mice/timepoint were anesthetized/euthanized for blood collection (plasma) and bronchoalveolar lavage fluid (BALF) collection with 2×0.5 mL flushes of saline. Urea levels for plasma/BALF normalization for the volume of lung epithelial lining fluid (ELF) calculation were quantified using a commercially-available spectrophotometry-based assay. CD101 concentrations in plasma/BALF samples were measured by LC with electrospray ionization tandem mass spectrometric (LC-MS/MS) detection.

Disseminated aspergillosis: ICR mice (6/grp) were made neutropenic by cyclophosphamide oπ Days −3 (270 mg/kg), +1 and +4 (90 mg/kg). IV infection with A. fumigatus ATCC 13073 (10⁴ CFU/mouse) on Day 0. Treatment (2 h after infection) with CD101 as a single dose (2 mg/kg IV and IP) or daily (0.5 mg/kg BID) dosing. Survival monitored for ≥10 days. Same model was used for prophylaxis except CD101 was dosed on Days −1, −3 or −5.

Pulmonary aspergillosis: ICR mice (10/grp) were made neutropenic by cyclophosphamide on Day −4 (150 mg/kg), and CPM/cortisone on Day −1 (150/175 mg/kg). Intranasal infection with A. fumigatus AF293 (10⁵ CFU/mouse) on Day 0. Prophylaxis CD101 as a single dose (IP; 5, 10, 20 mg/kg) or posaconazole (PO; 2 and 10 mg/kg) 1 day prior to infection. Survival monitored for 10 days.

### Results

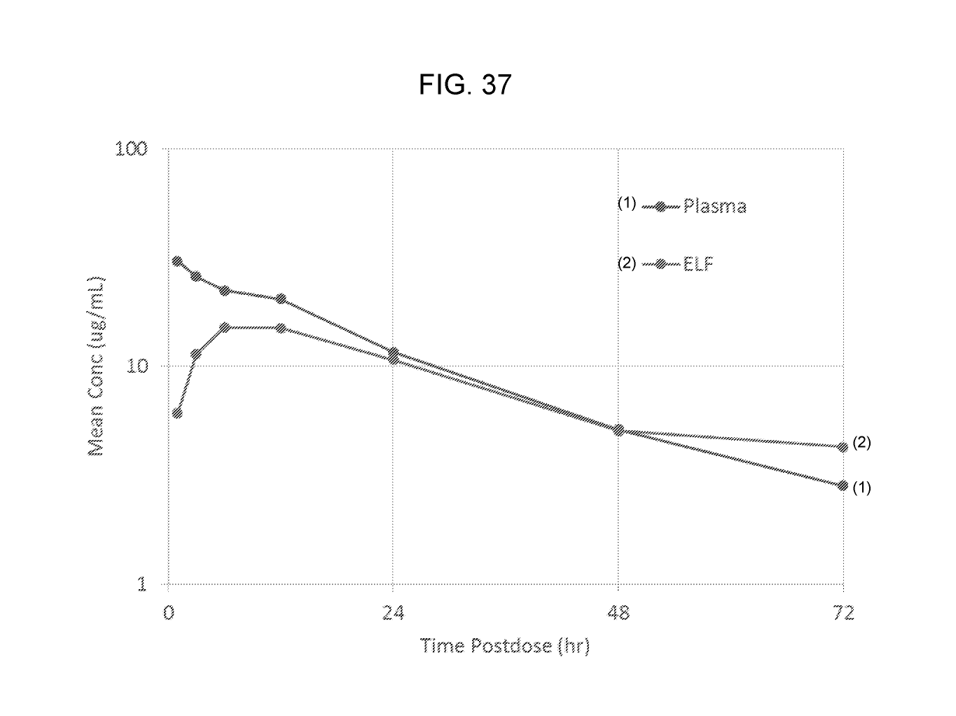

CD101 ELF concentrations reached a maximum by 4 hours and were comparable between plasma and ELF at 24 and 48 hours (FIG. 37). Concentration may potentially be higher in ELF by 72 hours suggesting possibly a longer half-life in ELF of 32 hour vs. 21 hour in plasma. Following CD101 administration, the mean maximum plasma concentration measured was 30.1 μg/mL and was observed at 1 hour post-dose, which was the first collection timepoint. The corresponding mean plasma AUC₀₋₇₂ and AUC were 762 and 848 μg·hr/mL, respectively, with a half-life of 21.1 hours. The mean maximum ELF concentration measured was 15.1 μg/mL, which was reached at 6 hours post-dose. Corresponding mean ELF AUC₀₋₇₂ and AUC were 606 and 802 μg·hr/mL, respectively, with a half-life of 31.9 hours. Based on AUC exposure ratios of ELF/plasma, the distribution of CD101 from plasma into lung ELF is close to unity (0.80 to 0.95).

For treatment of disseminated aspergillosis, CD101 by IV/IP at 0.2, 1, or 5 mg/kg BID×5d showed a significant increase in survival compared to vehicle. Survival was comparable when given either a single 2 mg/kg or as 0.2 mg/kg BID×5d dose. For prophylaxis, a single 5 mg/kg dose given up to 5 days prior to infection showed improved survival depending on day given. Doses 0 mg/kg showed 100% survival.

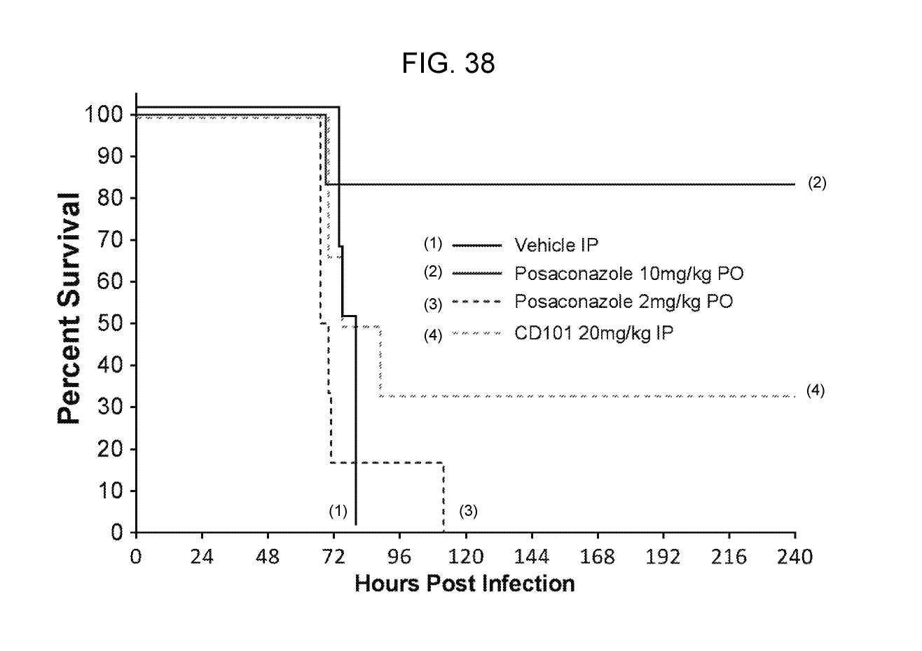

In the more challenging pulmonary aspergillosis model, dose-dependent increase in survival rate was observed from a single CD101 dose given one day prior to infection. The human (400 mg) AUC equivalent of 20 mg/kg in mice showed an increase in survival relative to control. Further comparison with posaconazole at the human AUC equivalent dose of 2 mg/kg (FIG. 38) suggests an advantage for CD101 with 30% survival rate compared to no survivors for posaconazole. Only posaconazole at 10 mg/kg showed a statistically-significant increase in survival rate relative to control.

### Example 18: High and Sustained CD101 Lung Epithelial Lining Fluid-to-Plasma Exposure Ratio from a Single Dose: Comparison to Posaconazole and Micafungin in a Mouse Pulmonary Aspergillosis Infection Model

CD101 has demonstrated in vitro potency and in vivo efficacy in mouse models of aspergillosis. The distribution of CD101 into lung ELF was studied to further substantiate this observed efficacy.

### Methods

Mice were dosed with CD101 (IP; 20 mg/kg) and then sacrificed for plasma and bronchoalveolar lavage fluid (BALF) collection between 0-72 hours. Urea for plasma/BALF normalization for ELF volume were quantified using spectrophotometry. CD101 concentrations in plasma/BALF samples were measured by LC-MS/MS. Total plasma concentrations were corrected for protein binding (99.2%).

Pulmonary aspergillosis: ICR mice (10/grp) were made neutropenic by cyclophosphamide on Day −4 (150 mg/kg), and cyclophosphamide/cortisone was given on Day −1 (150/175 mg/kg). Intranasal challenge with A. fumigatus AF293 (10⁵ CFU/mouse) was initiated on Day 0 and prophylaxis with CD101 as a single dose (IP; 5, 10, 20 mg/kg) or posaconazole (PO; 2 and 10 mg/kg) was started 1 day prior to infection. Survival was monitored for 10 days.

### Results

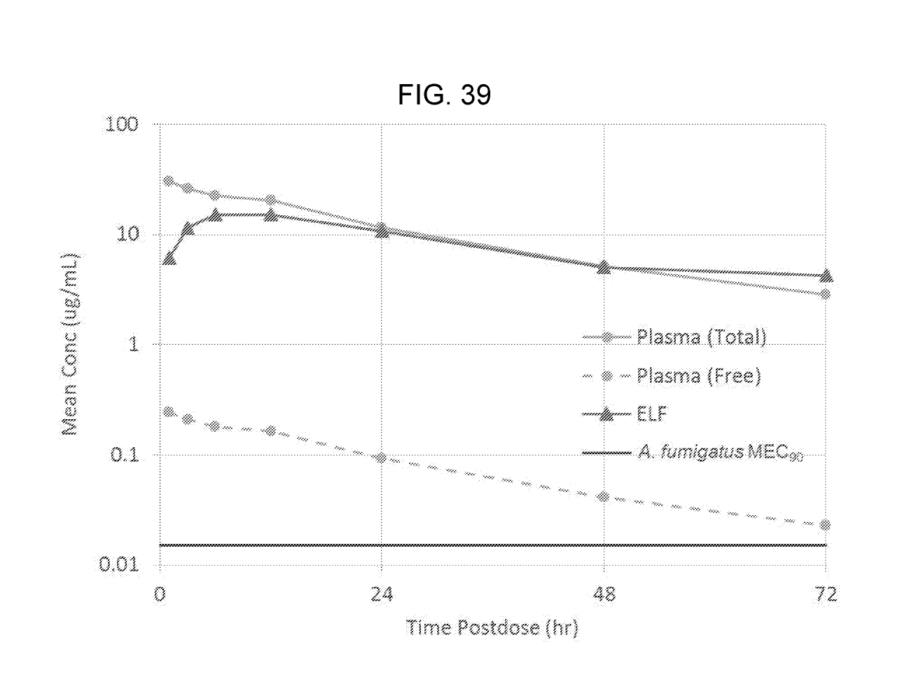

Maximum CD101 ELF concentrations were observed at 4 h and were comparable between plasma and ELF by 24 h post-dose as total-drug concentrations. At 72 h, mean ELF concentration (4 μg/mL) remained considerably higher than A. fumigatus MEC₉₀ of 0.015 μg/mL (FIG. 39). The resulting ELF/Plasma AUCₗₐₛₜ ratio was 0.80 for total-drug and 100 for free-drug exposures, respectively.

Dose-dependent increase in survival was observed from a single prophylaxis CD101 dose. The human (400 mg) AUC equivalent of 20 mg/kg in mice showed an increase in survival relative to control. CD101 protein binding results show a higher free fraction (˜3×) in human vs mouse plasma suggesting a lower human dose may be equally protective. Further comparison with posaconazole at the human-equivalent dose of 2 mg/kg or micafungin human-equivalent dose (100 mg) of 5 mg/kg in mice suggests an advantage for CD101 with 30% survival rate compared to no survivors for posaconazole or micafungin. Only posaconazole at 10 mg/kg (5×higher than human AUC) showed a statistically-significant increase in survival rate relative to control.

### Example 19: Assessment of the Efficacy of CD101 and Comparators in a Murine Model of Pulmonary Aspergillosis

The overall aim of the studies was to assess the antifungal efficacy of CD101 by intraperitoneal administration in a murine model of pulmonary aspergillosis caused by Aspergillus fumigatus strain AF293 (A. fumigatus AF293) compared to comparators posaconazole and micafungin. The primary objective of the study was to compare survival between the treatment groups. The secondary objective was to compare lung burden in vehicle and test article treated animals.

### Methods

Animal Strain and Housing.

Mice used in these studies were supplied by Charles River (Margate UK) and were specific pathogen free. The strain of mice used was ICR (also known as CD1 Mice) which is a well characterized outbred murine strain. Male mice were 11-15 g on receipt and were allowed to acclimatize for at least 7 days.

Immunosuppression.

Mice were immunosuppressed on Day −4 with 150 mg/kg cyclophosphamide administered intraperitoneally (IP), and on Day −1 with 150 mg/kg cyclophosphamide IP and 175 mg/kg cortisone acetate administered subcutaneously (SC). To prevent bacterial infection due to the immunosuppression mice were given once daily 50 mg/kg ceftazidime.

Preparation of Organism and Infection.

A. fumigatus strain AF293 inoculum was prepared from spore cultures grown on Sabouraud Dextrose agar (SAB) containing 50 μg/mL chloramphenicol (SABC) in vented tissue culture flasks. Following incubation for 7-10 days at 30° C., spore cultures were washed in sterile phosphate buffered saline (PBS) containing 0.05% Tween 80. Spore count was determined using a haemocytometer and spores were diluted in PBS to ˜6.9×10⁶ CFU/mL. Inoculum concentration was confirmed by quantitative culture onto SABC agar.

Neutropenic mice lungs were infected with 0.04 mL (0.02 mL/nare) of ˜4.17×106 CFU/mL (˜1.67×10⁶ CFU/mouse) of A. fumigatus AF293 by intranasal (IN) instillation under temporary 2.5% isoflurane induced anesthesia.

Preparation of Test Articles.

Micafungin (Mycamine, Astellas) was provided as a 50 mg vial (Lot 02323002, expiry August 2017) and was prepared as per manufacturer's instructions by adding 5 mL saline for injection (SFI) directly into the vial to make up a 10 mg/mL stock solution. This solution was then diluted further in SFI to a working concentration of 0.5 mg/mL. The compound was administered IP at 10 mL/kg to achieve a 2 mg/kg dose. It was prepared fresh once and stored at 4° C. until required.

Posaconazole (Noxafil 40 mg/mL oral suspension, Merck Sharp & Dohme Limited) was provided as a 40 mg/mL oral suspension (Lot N00801, expiry April 2019). This suspension was then diluted further in water for infection (WFI) to a working concentration of 0.2 and 1 mg/mL. The suspension was administered orally (PO) at 10 mL/kg for 2 and 10 mg/kg doses respectively, was prepared fresh once and stored at 4° C. until required.

Vehicle and CD101 diluent was 10% DMSO/1% Tween 20 in SFI: 1 mL of Tween 20 was added to 10 mL DMSO, gently mixed and SFI added to a final volume of 100 mL. This was filter sterilized and maintained at room temperature before use for dosing or formulating CD101. The vehicle was administered IP at 10 mL/kg.

Test article CD101 stock was prepared at 6 mg/mL in 10% DMSO/1% Tween 20 diluent. A clear non particulate solution was obtained following gentle mixing. Study doses of 20 mg/kg (2 mg/mL) were prepared from the 6 mg/mL stock as required by diluting in 10% DMSO/1% Tween 20 diluent. The 6 mg/mL stock was used undiluted for the 60 mg/kg study dose. All doses were administered IP at 10 mL/kg. The study doses were kept at 4° C. until required.

Treatment.

For this study, treatments were initiated on Day 1 pre infection according to treatment groups outlined in Table 22. A total of 36 mice (6/treatment group) were used in the study.

TABLE 22
Murine model of pulmonary aspergillosis treatment groups

| | Test | | | Dosing | Conc. | Dosage | | Total | Mice | End of |
|---|---|---|---|---|---|---|---|---|---|---|
| Group | Article | Route | Schedule | Day | mg/mL | ml/kg | mg/kg | Dose | (ICR) | study~ |
| 1 | Vehicle | IP | Single | −1 | — | — | — | — | 6 | 10 |
| 2 | Posaconazole | PO | Single | −1 | 1 | 10 | 10 | 2 | 6 | 10 |
| 3 | Posaconazole | PO | Single | −1 | 0.2 | 10 | 2 | 2 | 6 | 10 |
| 4 | Mica-fungin | IP | Single | −1 | 0.5 | 10 | 5 | 5 | 6 | 10 |
| 5 | CD101 | IP | Single | −1 | 2 | 10 | 20 | 20 | 6 | 10 |
| 6 | CD101 | IP | Single | −1 | 6 | 10 | 60 | 60 | 6 | 10 |

General Health Monitoring.

The mice were monitored at a frequency appropriate for their clinical condition. Mouse weights were recorded at least once daily both to ensure animals remained within ethical limits and to monitor efficacy of treatment.

Endpoints.

The primary endpoint for this study was survival within agreed ethical limits (>20% weight loss, severe hypothermia <34° C., inability to reach food or drink, severe hunching). Mice were monitored by daily weight measurements with observations as frequently as clinical condition required.

Mice presenting with severe clinical deterioration were humanely euthanized using an overdose of pentobarbitone administered by IP injection following clinical assessment and the time of death was recorded. Animal carcasses were stored at −20° C. before quantitative assessment of burden.

Ten days post infection all surviving animals were weighed and had their clinical condition assessed prior to being euthanized. Final survival numbers were recorded and analysed as described below and carcasses frozen at −20° C. prior to further processing.

Lung Burden.

A secondary endpoint for the study was terminal lung tissue burden. Immediately following confirmation of death, carcasses were frozen at −20° C. prior to tissue dissection and processing. The frozen carcasses were thawed at room temperature, the lungs removed and placed into pre-weighed bead-beating tubes containing 2 mL of PBS and subjected to mechanical disruption. Organ homogenates were diluted further in PBS and quantitatively cultured for A. fumigatus onto SABC and incubated at 30° C. for 24-48 hours.

In addition, a 300 μL aliquot of the undiluted lung tissue homogenate was stored at −80° C. for possible optional assessment of burden by qPCR.

Statistical Analysis.

Data were analysed using StatsDirect software (version 2.7.8):

1. Survival data were analysed using the Kaplan Meier and Log-Rank and Wilcoxon tests (using the Peto-Prentice weighting method).

2. Lung tissue burden data were analysed using the non-parametric Kruskal-Wallis test and if this was statistically significant all pairwise comparisons were analyzed (Conover-Inman).

### Results

The aim of this study was to determine the in vivo efficacy of CD101 and comparators in a murine model of pulmonary aspergillosis. The design of this study is summarized in Table 22. All treatments were well tolerated with no adverse signs observed.

Body Weights.

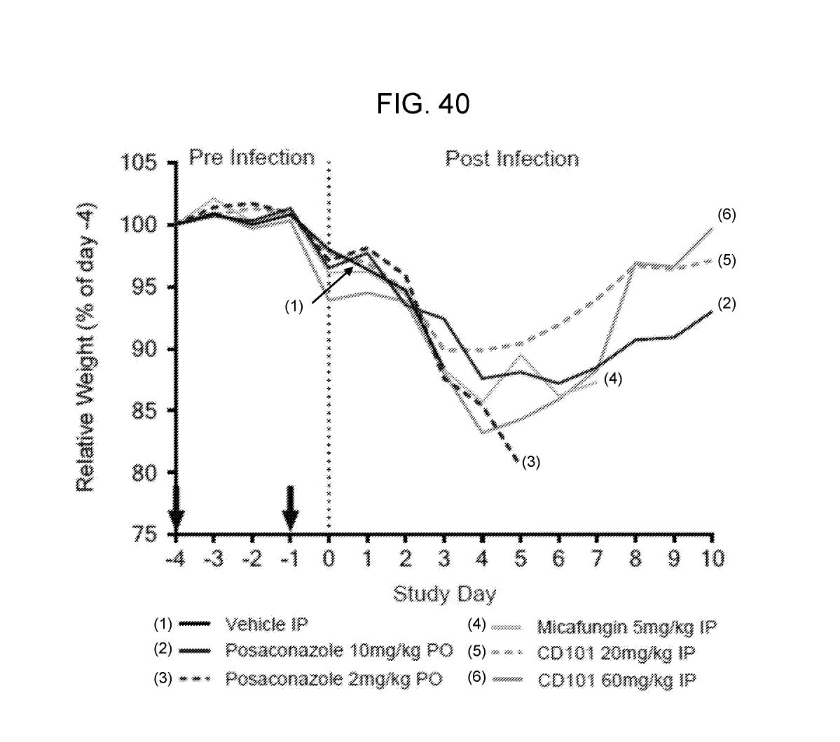

Animal weights following infection with A. fumigatus AF293 are shown in FIG. 40. Animal weights are shown relative to the weight on Day 4 pre infection.

Weights remained stable up to Day 1 pre-infection. Mice from all treatment groups lost weight following the immunosuppression on Day −1. The weight loss continued after the infection in almost all treatments groups up to Day 5 post infection; thereafter any mice that survived gained weight.

Survival.

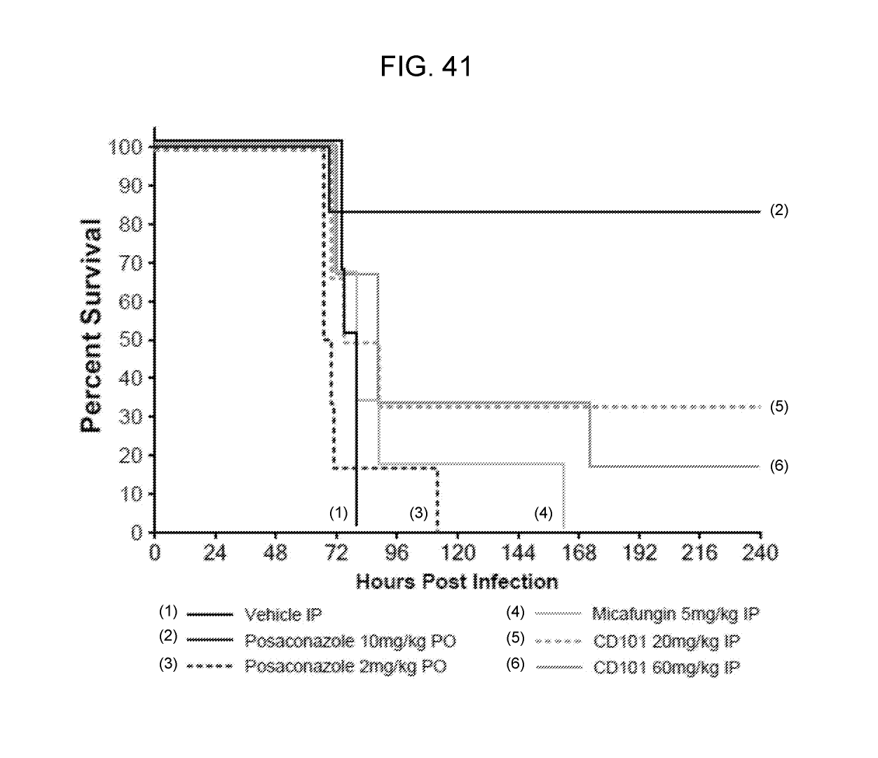

The median and mean survival for the various treatments are shown in Table 23, the survival plots in FIG. 41. Statistical outcomes from the Log-Rank and Wilcoxon test are shown in Table 24.

A robust survival model of pulmonary aspergillosis infection with A. fumigatus AF293 was established, with vehicle treated mice having a mean survival time of ˜77 h and a median survival time of ˜75 h post infection (range 74-80 h post infection). The study was terminated 10 days post infection as most mice had succumbed to the infection except in the 10 mg/kg posaconazole treatment group.

Treatment with test articles showed the following.
20 mg/kg CD101 dosed IP once on 1 day pre-infection—Mice had a longer mean survival time of ˜130 h compared to the vehicle treated mice but a similar median survival time of ˜75 h post infection (range 70-240 h). However, this was not statistically better than vehicle treated mice (FIG. 41, Tables 23 and 24). Two mice survived to the end of the study.
60 mg/kg CD101 dosed IP once on 1 day pre-infection—Mice had a longer mean and median survival time post infection compared to vehicle treated mice (˜123 h and ˜89 h respectively, range 73-240). A single mouse survived to the end of the study. Treatment with 60 mg/kg of CD101 did not result in significantly better survival compared to vehicle treated mice (FIG. 41, Tables 23 and 24).
Micafungin dosed at 5 mg/kg IP once on Day 1 pre-infection—Mice had a slightly longer mean and median survival time post infection compared to the vehicle treated mice (92 h and 80 h respectively, range 71-162 h) however, this was not statistically significant (FIG. 41, Tables 23 and 24).
Posaconazole dosed at 2 mg/kg PO once on Day 1 pre-infection—Mice had a similar mean survival time of 69 h and a median survival time of ˜78 h post infection (range 69-114 h). Statistically this was similar to the vehicle treated mice using the log-rank test but statistically lower survival compared to vehicle using the generalized Wilcoxon test (FIG. 41, Tables 23 and 24).
Posaconazole dosed at 10 mg/kg PO once on Day 1 pre-infection—Mice had a much longer mean survival time of 212 h post infection and median survival that could not be estimated as 5 mice survived to the end of the study (range 69-240 h). Statistically this was better than vehicle treated mice using the both the log-rank test and the generalized Wilcoxon test (FIG. 41, Tables 23 and 24).

TABLE 23
Mean and median survival per treatment group

| | Median Survival | Mean Survival |
|---|---|---|
| Treatment | (Hours) | (Hours) |
| Vehicle IP | 75.3 | 77.4 |
| Posaconazole 10 mg/kg PO | cannot estimate | 211.5 |
| Posaconazole 2 mg/kg PO | 69.0 | 77.5 |
| Micafungin 5 mg/kg IP | 80.0 | 92.0 |
| CD101 20 mg/kg IP | 74.7 | 130.4 |
| CD101 60 mg/kg IP | 88.7 | 122.7 |

TABLE 24
Log-Rank and Wilcoxon test output for different comparisons

| | | Wilcoxon (Peto- |
|---|---|---|
| Comparison | Log-Rank | Prentice) |
| Vehicle vs. Posaconazole 10 mg/kg | P = 0.0182 | P = 0.0441 |
| Vehicle vs. Posaconazole 2 mg/kg | NS | P = 0.0391 |
| Vehicle vs. Micafungin 5 mg/kg | NS | NS |
| Vehicle vs. CD101 20 mg/kg | NS | NS |
| Vehicle vs. CD101 60 mg/kg | NS | NS |

NS—not significant

A small satellite study looking at the effect of immunosuppression (n=6 mice) was running with one week delay and a different batch of mice. Two mice in the study were lost several days after the Day −1 combination immunosuppression (cyclophosphamide and cortisone acetate), the remaining four mice in the study survived to the end of the study. The loss of the two mice was most likely due to secondary infection due to Pseudomonas aeruginosa, the source of which is not clear.

The main study data are unlikely to be affected by secondary infections as the positive control included, posaconazole, showed good efficacy against the infection in line with expectations based on previous results.

Lung Burden

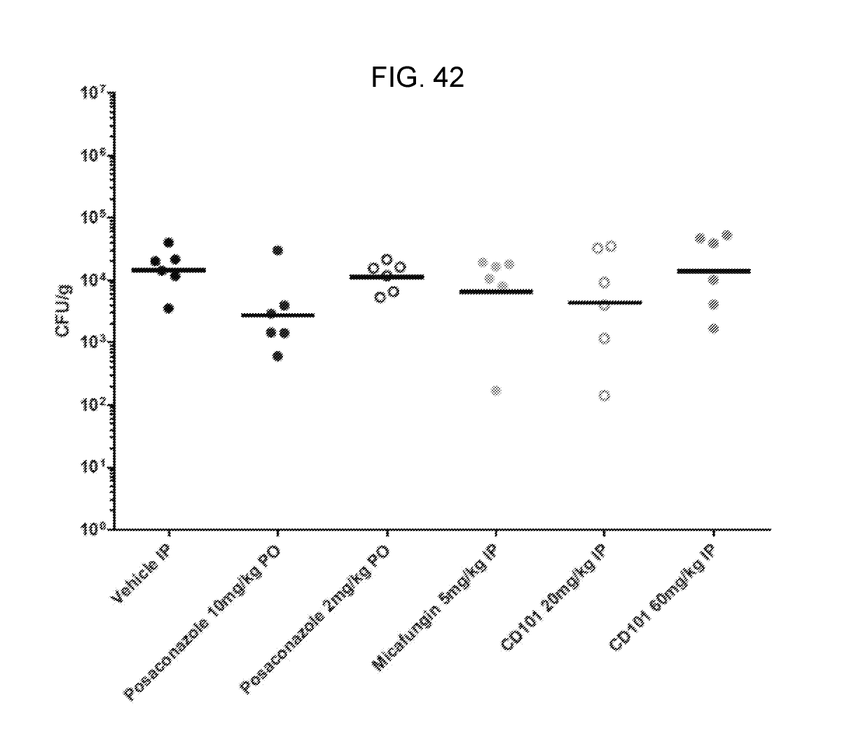

Terminal lung burden is shown in Table 25 and FIG. 42.

TABLE 25
Lung burden

| | | | | Log₁₀ |
|---|---|---|---|---|
| | Group | | Log₁₀ Group | reduction |
| | Geometric | Standard | Geometric | from vehicle |
| | mean | Deviation | mean | control |
| Treatment | (CFU/g) | (CFU/g) | (CFU/g) | (CFU/g) |
| Vehicle IP | 1.48 × 10⁴ | 1.24 × 10⁴ | 4.17 | 0.00 |
| Day −1 | | | | |
| Posaconazole | 2.75 × 10³ | 1.15 × 10⁴ | 3.44 | 0.73 |
| 10 mg/kg PO | | | | |
| Day −1 | | | | |
| Posaconazole | 1.14 × 10⁴ | 6.17 × 10³ | 4.06 | 0.11 |
| 2 mg/kg PO | | | | |
| Day −1 | | | | |
| Micafungin | 6.61 × 10³ | 7.34 × 10³ | 3.82 | 0.35 |
| 5 mg/kg IP | | | | |
| Day −1 | | | | |
| CD101 20 mg/ | 4.37 × 10³ | 1.60 × 10⁴ | 3.64 | 0.53 |
| kg IP | | | | |
| Day −1 | | | | |
| CD101 60 mg/ | 1.38 × 10⁴ | 2.30 × 10⁴ | 4.14 | 0.03 |
| kg IP | | | | |
| Day −1 | | | | |

### Conclusion

In this model of pulmonary aspergillosis, mice developed a robust infection with vehicle treated mice succumbing to the infection by Day 4 post infection. CD101 administered at 20 and 60 mg/kg once one day pre-infection resulted in slight increase in survival, which was statistically longer than the vehicle treatment. The comparator micafungin dosed at 5 mg/kg once one day pre-infection did not show any improvement in survival, with all mice succumbing to the infection by Day 7 post infection. The comparator posaconazole dosed at 2 mg/kg once one day pre-infection did not show any improvement in survival compared to the vehicle mice, with all mice succumbing to the infection by Day 6 post infection. Increasing the dose of posaconazole to 10 mg/kg and dosed once 1 day pre-infection resulted in >80% mice surviving to the end of the study, significantly longer than the vehicle control treatment.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present invention that come within known or customary practice within the art to which the invention pertains and may be applied to the essential features hereinbefore set forth, and follows in the scope of the claims.

Other embodiments are within the claims.

What is claimed is:

1. A method of treating a condition or disorder in a human subject comprising or consisting of administering to the subject a single dose of a pharmaceutical composition comprising CD101 salt, or a neutral form thereof, and one or more pharmaceutically acceptable excipients, wherein said single dose comprises an amount of the CD101 salt, or a neutral form thereof, sufficient to treat the condition or disorder.

2. A method of preventing a condition or disorder in a human subject comprising or consisting of administering to the subject a single dose of a pharmaceutical composition comprising CD101 salt, or a neutral form thereof, and one or more pharmaceutically acceptable excipients, wherein said single dose comprises an amount of the CD101 salt, or a neutral form thereof, sufficient to prevent the condition or disorder.

3. The method of claim 1 or 2, wherein the single dose is administered orally.

4. The method of claim 3, wherein the single dose comprises from 50 mg to 1200 mg of the CD101 salt, or a neutral form thereof.

5. The method of claim 1 or 2, wherein the single dose is administered intravenously.

6. The method of claim 5, wherein the single dose comprises from 50 mg to 1200 mg of the CD101 salt, or a neutral form thereof.

7. The method of claim 1 or 2, wherein the single dose is administered subcutaneously.

8. The method of claim 7, wherein the single dose comprises from 50 mg to 1200 mg of the CD101 salt, or a neutral form thereof.

9. The method of any one of claims 1-8, wherein the disease or condition is selected from the group consisting of candidemia, invasive candidiasis, onychomycosis, aspergillosis, and a Pneumocystis infection.

10. The method of claim 1-9, wherein the disease or condition is a fungal infection.

11. The method of claim 10, wherein the fungal infection is a Candida infection.

12. The method of claim 10, wherein the fungal infection is an Aspergillus infection.

13. The method of claim 10, wherein the fungal infection is a Pneumocystis infection.

14. The method of any one of claims 1-13, wherein the administration of the single dose substantially eliminates or prevents a fungal infection.

15. The method of any one of claims 1-14, wherein the subject does not receive any concurrent antifungal treatment.

16. The method of any one of claims 1-14, wherein the subject does not receive any antifungal treatment within 21 days following the administration of the pharmaceutical composition.

17. The method of any one of claims 1-16, wherein the pharmaceutical composition consists of the CD101 salt, or a neutral form thereof, and the one or more pharmaceutically acceptable excipients.

18. The method of any one of claims 1-17, wherein the condition or disorder is candidemia, oropharyngeal candidiasis, esophageal candidiasis, mucosal candidiasis, genital candidiasis, vulvovaginal candidiasis, rectal candidiasis, hepatic candidiasis, renal candidiasis, pulmonary candidiasis, splenic candidiasis, otomycosis, osteomyelitis, septic arthritis, cardiovascular candidiasis, or invasive candidiasis.

19. A method of preventing or treating a biofilm in a subject comprising administering to the subject a pharmaceutical composition comprising CD101 salt, or a neutral form thereof, and one or more pharmaceutically acceptable excipients.

20. The method of claim 19, wherein the biofilm is a Candida biofilm.

21. The method of claim 20, wherein the Candida biofilm is a Candida albicans biofilm or a Candida auris biofilm.

22. The method of any one of claims 19-21, wherein the biofilm is attached to a mucous membrane of the subject.

23. A method of preventing biofilm growth on a catheter or killing a biofilm attached to a catheter comprising submerging the catheter in an aqueous solution comprising CD101 salt, or a neutral form thereof, or running an aqueous solution comprising CD101 salt, or a neutral form thereof, through the lumen of the catheter.

24. The method of claim 23, wherein the biofilm is a Candida biofilm.

25. The method of claim 24, wherein the Candida biofilm is a Candida albicans biofilm or a Candida auris biofilm.


SEQUENCE LISTING (2 sequences)

SEQ ID NO: 1 - DNA, 20 bp, Artificial Sequence
  Synthetic Construct Cspp_F2275
aatgggctggtgctcaacat

SEQ ID NO: 2 - DNA, 26 bp, Artificial Sequence
  Synthetic Construct Cspp_R3070
ccttcaatttcagatggaacttgatg